(19) United States
(12) Patent Application Publication
(10) Pub. No.: US 2026/0308613 A1
(43) Pub. Date: Oct. 8, 2026

(54) COMPOSITIONS AND METHODS FOR THE TREATMENT OF NEUROMUSCULAR DISORDERS

(71) Applicant: ASTELLAS GENE THERAPIES INC., South San Francisco, CA (US)

(72) Inventors: Carlos FONCK, Lafayette, CA (US); Clarice Weili CHEN, Anaheim, CA (US); Dwaipayan SEN, San Bruno, CA (US)

(21) Appl. No.: 19/108,319

(22) PCT Filed: Sep. 22, 2023
(86) PCT No.: PCT/US2023/074909

§ 371 Date: Mar. 3, 2025

Related U.S. Application Data
(60) Provisional application No. 63/409,423, filed on Sep. 23, 2022.

(51) Int. Cl. C12N 15/86; A61K 38/00; A61K 48/00; A61P 21/00; C12N 9/16

(52) U.S. Cl. CPC C12N 15/86; A61K 48/0058; A61P 21/00; C12N 9/16; A61K 38/00; C12N 2750/14143; C12N 2830/008; C12Y 301/03064

(57) ABSTRACT

Some embodiments relate to adeno-associated virus (AAV)-mediated delivery of nucleic acids for treating neuromuscular disorders, e.g., X-linked myotubular myopathy (XLMTM), in patients in need thereof. The AAV vectors of the disclosure may include, e.g., a transgene encoding a myotubularin protein operably linked to one or more transcription regulatory elements. In some embodiments, the one or more transcription regulatory elements are specifically active in muscle and/or liver tissue.

### SEQUENCE LISTING

The instant application contains a Sequence Listing which has been submitted electronically in XML format and is hereby incorporated by reference in its entirety. Said XML copy, created on Sep. 14, 2023, is named “51037-075WO2_Sequence_Listing_9_14_23” and is 27,146 bytes in size.

### BACKGROUND OF THE INVENTION

X-linked myotubular myopathy (XLMTM) is a fatal monogenic disease of skeletal muscle, resulting from mutations in the myotubularin 1 (MTM1) gene. Approximately one in every 50,000 newborn boys has XLMTM, which typically displays as marked hypotonia and respiratory failure. In extremely rare cases, females can develop a severe form of XLMTM. Survival beyond the postnatal period requires intensive support, including respiratory support (i.e., mechanical ventilation) at birth in 85-90% of patients, ongoing 24-hour ventilator dependence in nearly 50% of patients, and tracheostomy in ~60% of patients. Until recently, only supportive treatment options, such as ventilator use or a feeding tube, were available. Recently, a gene therapy approach involving the delivery of MTM1 has been developed for the treatment of XLMTM. However, there is a need in the art for improved methods of administering gene therapy to patients having XLMTM.

### SUMMARY OF THE INVENTION

The present disclosure features compositions and methods useful for the treatment of neuromuscular disorders, particularly X-linked myotubular myopathy (XLMTM). Using the compositions and methods described herein, a patient (e.g., a human patient) having XLMTM may be administered a viral vector, such as an adeno-associated viral (AAV) vector, having a transgene encoding myotubularin 1 (MTM1). Exemplary composition and methods of the disclosure are described below.

In a first aspect, the disclosure provides a recombinant AAV vector comprising a transgene encoding MTM1, wherein the transgene is operably linked to a promoter that is active in liver tissue.

In some embodiments, the promoter is selectively active in liver tissue. In some embodiments, upon separately contacting the vector with one or more liver cells and one or more non-liver cells (e.g., in vitro or in vivo) under equivalent conditions, the vector results in a level of expression of the transgene in the one or more liver cells that is greater than the level of expression of the transgene in the one or more non-liver cells, for example, from 2-fold to 1,000-fold greater (e.g., 2-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, or 1,000-fold greater than the level of expression of the transgene in the one or more non-liver cells). In some embodiments, upon separately contacting the vector with the one or more liver cells and the one or more non-liver cells, the vector results in a level of expression of the transgene in the one or more liver cells that is from 10-fold to 1,000-fold greater than the level of expression of the transgene in the one or more non-liver cells. In some embodiments, upon separately contacting the vector with the one or more liver cells and the one or more non-liver cells, the vector results in a level of expression of the transgene in the one or more liver cells that is from 50-fold to 1,000-fold greater than the level of expression of the transgene in the one or more non-liver cells. In some embodiments, upon separately contacting the vector with the one or more liver cells and the one or more non-liver cells, the vector results in a level of expression of the transgene in the one or more liver cells that is from 100-fold to 1,000-fold greater than the level of expression of the transgene in the one or more non-liver cells. In some embodiments, upon separately contacting the vector with the one or more liver cells and the one or more non-liver cells, the vector results in a level of expression of the transgene in the one or more liver cells that is at least 2-fold, at least 5-fold, at least 10-fold, at least 50-fold, or at least 100-fold greater than the level of expression of the transgene in the one or more non-liver cells.

In some embodiments, the non-liver cells are muscle cells or neural cells. In some embodiments, the non-liver cells are cardiac cells.

In some embodiments, upon contacting the vector with one or more liver cells (e.g., of a patient having a neuromuscular disorder), the vector results in a level of expression of the transgene in the one or more liver cells that is closer to the expression of MTM1 in wild-type/healthy liver cells, for example, the expression of the transgene may be from 0.2-fold to 10-fold (e.g., 0.2-fold, 0.3-fold, 0.4-fold. 0.5-fold, 0.6-fold, 0.7-fold, 0.8-fold, 0.9-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold) the expression level of MTM1 in wild-type/healthy liver cells. In some embodiments, upon contacting the vector with one or more liver cells (e.g., of a patient having a neuromuscular disorder), the vector results in a level of expression of the transgene in the one or more liver cells that is at least 0.2-fold, at least 0.5-fold, at least 1-fold, at least 2-fold, at least 3-fold, at least 4-fold, at least 5-fold, at least 6-fold, at least 7-fold, at least 8-fold, at least 9-fold, or at least 10-fold the level of wild-type MTM1 expression in a healthy liver cell.

In some embodiments, the promoter comprises an LP1 promoter. In some embodiments, the LP1 promoter has a nucleic acid sequence that is at least 75% identical to the nucleic acid sequence of SEQ ID NO: 3:

(SEQ ID NO: 3)
GTCCCCTAAAATGGGCAAACATTGCAAGCAGCAAACAGCAAACACACAGC
CCTCCCTGCCTGCTGACCTTGGAGCTGGGGCAGAGGTCAGAGACCTCTCT
GGGCCCATGCCACCTCCAACATCCACTCGACCCCTTGGAATTTCGGTGGA
GAGGAGCAGAGGTTGTCCTGGCGTGGTTTAGGTAGTGTGAGAGGGGAATG
ACTCCTTTCGGTAAGTGCAGTGGAAGCTGTACACTGCCCAGGCAAAGCGT
CCGGGCAGCGTAGGCGGGCGACTCAGATCCCAGCCAGTGcACTTAGCCCC
TGTTTGCTCCTCCGATAACTGGGGTGACCTTGGTTAATATTCACCAGCAG
CCTCCCCCGTTGCCCCTCTGGATCCACTGCTTAAATACGGACGAGGACAG
GGCCCTGTCTCCTCAGCTTCAGGCACCACCACTGACCTGGGACAGTGAA.

In some embodiments, the LP1 promoter has a nucleic acid sequence that is at least 80% identical to the nucleic acid sequence of SEQ ID NO: 3 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 3). In some embodiments, the LP1 promoter has the nucleic acid sequence of SEQ ID NO: 3.

In some embodiments, the promoter comprises an apolipoprotein E (ApoE) promoter. In some embodiments, the ApoE promoter has a nucleic acid sequence that is at least 75% identical to the nucleic acid sequence of SEQ ID NO: 8:

(SEQ ID NO: 8)
CCCTAAAATGGGCAAACATTGCAAGCAGCAAACAGCAAACACACAGCCCT
CCCTGCCTGCTGACCTTGGAGCTGGGGCAGAGGTCAGAGACCTCTCTGGG
CCCATGCCACCTCCAACATCCACTCGACCCCTTGGAATTTCGGTGGAGAG
GAGCAGAGGTTGTCCTGGCGTGGTTTAGGTAGTGTGAGAGGGG.

In some embodiments, the ApoE promoter has a nucleic acid sequence that is at least 80% identical to the nucleic acid sequence of SEQ ID NO: 8 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments, the ApoE promoter has the nucleic acid sequence of SEQ ID NO: 8.

In some embodiments, the promoter comprises an alpha-1-antitrysin (A1AT) promoter. In some embodiments, the A1AT promoter has a nucleic acid sequence that is at least 75% identical to the nucleic acid sequence of SEQ ID NO: 9:

(SEQ ID NO: 9)
AATGACTCCTTTCGGTAAGTGCAGTGGAAGCTGTACACTGCCCAGGCAAA
GCGTCCGGGCAGCGTAGGCGGGCGACTCAGATCCCAGCCAGTGGACTTAG
CCCCTGTTTGCTCCTCCGATAACTGGGGTGACCTTGGTTAATATTCACCA
GCAGCCTCCCCCGTTGCCCCTCTGGATCCACTGCTTAAATACGGACGAGG
ACAGGGCCCTGTCTCCTCAGCTTCAGGCACCACCACTGACCTGGGACAGT
GAAT.

In some embodiments, the A1AT promoter has a nucleic acid sequence that is at least 80% identical to the nucleic acid sequence of SEQ ID NO: 9 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 9). In some embodiments, the A1AT promoter has the nucleic acid sequence of SEQ ID NO: 9.

In some embodiments, the promoter is a chimeric promoter comprising an ApoE promoter and an A1AT promoter. In some embodiments, the chimeric promoter has a nucleic acid sequence that is at least 75% identical to the nucleic acid sequence of SEQ ID NO: 2:

(SEQ ID NO: 2)
CGTGATCTAGTAGGCTCAGAGGCACACAGGAGTTTCTGGGCTCACCCTGC
CCCCTTCCAACCCCTCAGTTCCCATCCTCCAGCAGCTGTTTGTGTGCTGC
CTCTGAAGTCCACACTGAACAAACTTCAGCCTACTCATGTCCCTAAAATG
GGCAAACATTGCAAGCAGCAAACAGCAAACACACAGCCCTCCCTGCCTGC
TGACCTTGGAGCTGGGGCAGAGGTCAGAGACCTCTCTGGGCCCATGCCAC
CTCCAACATCCACTCGACCCCTTGGAATTTCGGTGGAGAGGAGCAGAGGT
TGTCCTGGCGTGGTTTAGGTAGTGTGAGAGGGGTACCCGGGGATOTTGCT
ACCAGTGGAACAGCCACTAAGGATTCTGCAGTGAGAGCAGAGGGCCAGCT
AAGTGGTACTCTCCCAGAGACTGTCTGACTCACGCCACCCCCTCCACCTT
GGACACAGGACGCTGTGGTTTCTGAGCCAGGTACAATGACTCCTTTCGGT
AAGTGCAGTGGAAGCTGTACACTGCCCAGGCAAAGCGTCCGGGCAGCGTA
GGCGGGCGACTCAGATCCCAGCCAGTGGACTTAGCCCCTGTTTGCTCCTC
CGATAACTGGGGTGACCTTGGTTAATATTCACCAGCAGCCTCCCCCGTTG
CCCCTCTGGATCCACTGCTTAAATACGGACGAGGACAGGGCCCTGTCTCC
TCAGCTTCAGGCACCACCACTGACCTGGGACAGTGAATGATCCCCCTGAT
CTGCGGCCTCGACGGTATCGATAAG.

In some embodiments, the chimeric promoter has a nucleic acid sequence that is at least 80% identical to the nucleic acid sequence of SEQ ID NO: 2 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the chimeric promoter has the nucleic acid sequence of SEQ ID NO: 2.

In some embodiments, the promoter comprises a constitutive promoter. In some embodiments, the constitutive promoter is a phosphoglycerate kinase (PGK) promoter, an elongation factor-1 alpha (EF1 alpha) promoter, a glyceraldehyde 3-phosphate dehydrogenase (GAPDH) promoter, a cytomegalovirus (CMV) promoter, or a chicken-β-actin (CBA) promoter.

In some embodiments, the AAV vector further comprises a second transgene encoding MTM1. In some embodiments, the second transgene encoding MTM1 is operably linked to a promoter that is active in muscle tissue. In some embodiments, the promoter that is active in muscle tissue is selectively active in muscle tissue.

In some embodiments, the promoter that is selectively active in muscle tissue effectuates (e.g., in vitro or in vivo) a level of expression of the MTM1 transgene in muscle cells that is greater than the level of expression of the MTM1 transgene effectuated by the same promoter in non-muscle cells, for example, from 2-fold to 1,000-fold greater (e.g., 2-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, or 1,000-fold greater than the level of expression of the MTM1 transgene effectuated by the same promoter in non-muscle cells). In some embodiments, the promoter that is selectively active in muscle tissue effectuates a level of expression of the MTM1 transgene in muscle cells that is from 10-fold to 1,000-fold greater than the level of expression of the MTM1 transgene effectuated by the same promoter in non-muscle cells. In some embodiments, the promoter that is selectively active in muscle tissue effectuates a level of expression of the MTM1 transgene in muscle cells that is from 50-fold to 1,000-fold greater than the level of expression of the MTM1 transgene effectuated by the same promoter in non-muscle cells. In some embodiments, the promoter that is selectively active in muscle tissue effectuates a level of expression of the MTM1 transgene in muscle cells that is from 100-fold to 1,000-fold greater than the level of expression of the MTM1 transgene effectuated by the same promoter in non-muscle cells. In some embodiments, the promoter that is selectively active in muscle tissue effectuates a level of expression of the MTM1 transgene in muscle cells that is at least 2-fold, at least 5-fold, at least 10-fold, at least 50-fold, or at least 100-fold greater than the level of expression of the MTM1 transgene effectuated by the same promoter in non-muscle cells.

In some embodiments, upon contacting the vector with one or more liver cells (e.g., of a patient having a neuromuscular disorder, the vector results in a level of expression of the transgene in the one or more liver cells that is closer to the expression of MTM1 in wild-type/healthy liver cells, for example, the expression of the transgene may be from 0.2-fold to 10-fold (e.g., 0.2-fold, 0.3-fold, 0.4-fold. 0.5-fold, 0.6-fold, 0.7-fold, 0.8-fold, 0.9-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold) the expression level of MTM1 in wild-type/healthy liver cells. In some embodiments, upon contacting the vector with one or more liver cells (e.g., of a patient having a neuromuscular disorder), the vector results in a level of expression of the transgene in the one or more liver cells that is at least 0.2-fold, at least 0.5-fold, at least 1-fold, at least 2-fold, at least 3-fold, at least 4-fold, at least 5-fold, at least 6-fold, at least 7-fold, at least 8-fold, at least 9-fold, or at least 10-fold the level of wild-type MTM1 expression in a healthy liver cell.

In some embodiments, the second transgene encoding MTM1 is operably linked to a muscle creatine kinase (MCK) promoter or a desmin (DES) promoter.

In some embodiments, each transgene encoding MTM1, independently, has a nucleic acid sequence that is at least 75% identical to the nucleic acid sequence of residues 4927-6748 of SEQ ID NO: 1. In some embodiments, each transgene encoding MTM1, independently, has a nucleic acid sequence that is at least 80% identical to the nucleic acid sequence of residues 4927-6748 of SEQ ID NO: 1 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of residues 4927-6748 of SEQ ID NO: 1). In some embodiments, each transgene encoding MTM1 has the nucleic acid sequence of residues 4927-6748 of SEQ ID NO: 1.

In some embodiments, each transgene encoding MTM1, independently, has a nucleic acid sequence that is at least 75% identical to the nucleic acid sequence of SEQ ID NO: 6. In some embodiments, each transgene encoding MTM1, independently, has a nucleic acid sequence that is at least 80% identical to the nucleic acid sequence of SEQ ID NO: 6 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 6). In some embodiments, each transgene encoding MTM1 has the nucleic acid sequence of SEQ ID NO: 6.

In some embodiments, each transgene encoding MTM1, independently, has a nucleic acid sequence that is at least 75% identical to the nucleic acid sequence of SEQ ID NO: 7. In some embodiments, each transgene encoding MTM1, independently, has a nucleic acid sequence that is at least 80% identical to the nucleic acid sequence of SEQ ID NO: 7 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 7). In some embodiments, each transgene encoding MTM1 has the nucleic acid sequence of SEQ ID NO: 7.

In some embodiments, the MTM1 sequence is codon-optimized.

In some embodiments, the AAV is an AAV1, AAV2, AAV3, AAV4, AAV5, AAV6, AAV7, AAV8, AAV9, AAVrh10, or AAVrh74 serotype. In some embodiments, the AAV vector is a pseudotyped AAV. In some embodiments, the pseudotyped AAV is AAV2/8 or AAV2/9.

In another aspect, the disclosure features a method of treating XLMTM in a human patient in need thereof by administering to the patient a therapeutically effective amount of the AAV vector of any of the above aspects or embodiments of the disclosure.

In some embodiments, the patient is five years old or younger at the time of administration of the AAV vector. In some embodiments, the patient is four years old or younger at the time of administration of the AAV vector, optionally wherein the patient is three years old or younger, two years old or younger, one year old or younger, or six months old or younger.

In some embodiments, the AAV vector is administered to the patient in an amount of less than 3×10¹⁴ vg/kg. In some embodiments, the AAV vector is administered to the patient in an amount of less than 2.5×10¹⁴ vg/kg, optionally wherein the AAV vector is administered to the patient in an amount of less than 2×10¹⁴ vg/kg, less than 1.5×10¹⁴ vg/kg, or less than 1.4×10¹⁴ vg/kg. In some embodiments, the AAV vector is administered to the patient in an amount of from 3×10¹³ vg/kg to 2.3×10¹⁴ vg/kg, optionally wherein the AAV vector is administered to the patient in an amount of from 8×10¹³ vg/kg to 1.8×10¹⁴ vg/kg, from 1×10¹⁴ vg/kg to 1.6×10¹⁴ vg/kg, from 1.1×10¹⁴ vg/kg to 1.5×10¹⁴ vg/kg, or from 1.2×10¹⁴ vg/kg to 1.4×10¹⁴ vg/kg. In some embodiments, the AAV vector is administered to the patient in an amount of about 1.3×10¹⁴ vg/kg.

In some embodiments, the AAV vector is administered to the patient by way of intravenous, intramuscular, intrahepatic, intradermal, or subcutaneous administration.

In some embodiments, the patient is further administered an anti-cholestatic agent. In some embodiments, the anti-cholestatic agent is selected from the group consisting of a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor. In some embodiments: (i) the FXR ligand is obeticholic acid, cilofexor, tropifexor, tretinoin, or EDP-305; (ii) the FGF-19 mimetic is aldafermin; (iii) the TGR5 agonist is INT-777 or INT-767; (iv) the PPAR agonist is bezafibrate, fenofibrate, seladelpar, or elafibrinor; (v) the PPAR-alpha agonist is fenofibrate; (vi) the PPAR-delta agonist is seladelpar; (vii) the dual PPAR-alpha and PPAR-delta agonist is elafibranor; (viii) the ASBT inhibitor is odevixibat, maralixibat, or linerixibat; (ix) the immunomodulatory drug is rituximab, abatacept, ustekinumab, infliximab, baricitinib, or FFP-104; (x) the antifibrotic therapy is a vitamin D receptor agonist or simtuzumab; and/or (xi) the NOX inhibitor is setanaxib.

In some embodiments, the bile acid is ursodeoxycholic acid, nor-ursodeoxycholic acid, or a pharmaceutically acceptable salt thereof.

In some embodiments, the patient does not have a history of cholestasis or hyperbilirubinemia. In some embodiments, the patient does not have a history of any underlying liver disease.

In some embodiments, the method comprises administering to the patient therapeutically effective amounts of: (i) an AAV vector comprising a transgene encoding MTM1 under the control of a promoter that is active (e.g., selectively active) in liver tissue, and (ii) an AAV vector comprising a transgene encoding MTM1 under the control of a promoter that is active (e.g., selectively active) in muscle tissue.

In some embodiments, the promoter that is active in liver tissue comprises an LP1 promoter. In some embodiments, the LP1 promoter has a nucleic acid sequence that is at least 75% identical to the nucleic acid sequence of SEQ ID NO: 3. In some embodiments, the LP1 promoter has a nucleic acid sequence that is at least 80% identical to the nucleic acid sequence of SEQ ID NO: 3 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 3). In some embodiments, the LP1 promoter has the nucleic acid sequence of SEQ ID NO: 3.

In some embodiments, the promoter that is active in liver tissue comprises an ApoE promoter. In some embodiments, the ApoE promoter has a nucleic acid sequence that is at least 75% identical to the nucleic acid sequence of SEQ ID NO: 8. In some embodiments, the ApoE promoter has a nucleic acid sequence that is at least 80% identical to the nucleic acid sequence of SEQ ID NO: 8 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments, the ApoE promoter has the nucleic acid sequence of SEQ ID NO: 8.

In some embodiments, the promoter that is active in liver tissue comprises an A1AT promoter. In some embodiments, the A1AT promoter has a nucleic acid sequence that is at least 75% identical to the nucleic acid sequence of SEQ ID NO: 9. In some embodiments, the A1AT promoter has a nucleic acid sequence that is at least 80% identical to the nucleic acid sequence of SEQ ID NO: 9 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 9). In some embodiments, the A1AT promoter has the nucleic acid sequence of SEQ ID NO: 9.

In some embodiments, the promoter that is active in liver tissue is a chimeric promoter comprising an ApoE promoter and an A1AT promoter. In some embodiments, the chimeric promoter has a nucleic acid sequence that is at least 75% identical to the nucleic acid sequence of SEQ ID NO: 2. In some embodiments, the chimeric promoter has a nucleic acid sequence that is at least 80% identical to the nucleic acid sequence of SEQ ID NO: 2 (e.g., a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the chimeric promoter has the nucleic acid sequence of SEQ ID NO: 2.

In a further aspect, the disclosure features a method of treating XLMTM in a human patient in need thereof by administering to the patient therapeutically effective amounts of: (i) a non-viral composition comprising a nucleic acid encoding MTM1 operably linked to a promoter that is active (e.g., selectively active) in liver tissue, and (ii) an AAV vector comprising a transgene encoding MTM1 under the control of a promoter that is active (e.g., selectively active) in muscle tissue.

In some embodiments, the non-viral composition is a liposome, vesicle, synthetic vesicle, exosome, synthetic exosome, dendrimer, or nanoparticle. In some embodiments, the nanoparticle is a lipid nanoparticle, which can deliver DNA constructs similar in composition and size to those delivered via AAV capsids.

In some embodiments, the promoter is a DES promoter. In some embodiments, the nucleic acid encoding MTM1 and the transgene encoding MTM1 each, independently, have a nucleic acid sequence that is at least 75% identical to the nucleic acid sequence of residues 4927-6748 of SEQ ID NO: 1. In some embodiments, the nucleic acid encoding MTM1 and the transgene encoding MTM1 each, independently, have a nucleic acid sequence that is at least 80% identical to the nucleic acid sequence of residues 4927-6748 of SEQ ID NO: 1. In some embodiments, the nucleic acid encoding MTM1 and the transgene encoding MTM1 each, independently, have a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of residues 4927-6748 of SEQ ID NO: 1. In some embodiments, the nucleic acid encoding MTM1 and the transgene encoding MTM1 each have the nucleic acid sequence of residues 4927-6748 of SEQ ID NO: 1.

In some embodiments, the nucleic acid encoding MTM1 and the transgene encoding MTM1 each, independently, have a nucleic acid sequence that is at least 75% identical to the nucleic acid sequence of SEQ ID NO: 6. In some embodiments, the nucleic acid encoding MTM1 and the transgene encoding MTM1 each, independently, have a nucleic acid sequence that is at least 80% identical to the nucleic acid sequence of SEQ ID NO: 6. In some embodiments, the nucleic acid encoding MTM1 and the transgene encoding MTM1 each, independently, have a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 6.

In some embodiments, the nucleic acid encoding MTM1 and the transgene encoding MTM1 each have the nucleic acid sequence of SEQ ID NO: 6.

In some embodiments, the nucleic acid encoding MTM1 and the transgene encoding MTM1 each, independently, have a nucleic acid sequence that is at least 75% identical to the nucleic acid sequence of SEQ ID NO: 7. In some embodiments, the nucleic acid encoding MTM1 and the transgene encoding MTM1 each, independently, have a nucleic acid sequence that is at least 80% identical to the nucleic acid sequence of SEQ ID NO: 7. In some embodiments, the nucleic acid encoding MTM1 and the transgene encoding MTM1 each, independently, have a nucleic acid sequence that is at least 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 7. In some embodiments, the nucleic acid encoding MTM1 and the transgene encoding MTM1 each have the nucleic acid sequence of SEQ ID NO: 7.

In some embodiments, the AAV vector is resamirigene bilparvovec.

In a further aspect, the disclosure provides a kit comprising the AAV vector of any of the above aspects or embodiments of the disclosure. The kit may further include a package insert instructing a user to administer the AAV vector to a patient diagnosed as having XLMTM.

In another aspect, the disclosure provides a kit comprising: (i) a non-viral composition comprising a nucleic acid encoding MTM1 (e.g., a non-viral composition described above), and (ii) an AAV vector comprising a transgene encoding MTM1 under the control of a promoter that is active (e.g., selectively active) in muscle tissue (e.g., an AAV vector described above). The kit may further include a package insert instructing a user to administer the non-viral composition and the AAV vector to a patient diagnosed as having XLMTM.

### BRIEF DESCRIPTION OF THE DRAWINGS

FIG. 1 is a schematic drawing of an exemplary adeno-associated virus (AAV) vector for the expression of the human or mouse myotubularin 1 (MTM1) gene (e.g., a pseudotyped AAV vector, such as an AAV vector containing AAV2 inverted terminal repeats packaged within capsid proteins from AAV8). From left to right, the shaded arrows and rectangles represents the nucleic acid sequences encoding an ApoE/A1AT liver-specific promoter (ApoE/A1AT) operatively linked to a Beta-globin Intron (b-globin intron), a human or mouse MTM1 gene, an SV40 poly-adenylation signal (SV40-polyA), and flanking inverted terminal repeat sequences (ITR).

FIG. 2 is a schematic drawing of an exemplary AAV vector for the expression of the human or mouse myotubularin 1 (MTM1) gene (e.g., a pseudotyped AAV vector, such as an AAV vector containing AAV2 inverted terminal repeats packaged within capsid proteins from AAV8). From left to right, the shaded arrows and rectangles represent the nucleic acid sequences encoding a LP1 liver-specific promoter (LP1 promoter) operatively linked to an SV40 Intron, a human or mouse MTM1 gene, an SV40 poly-adenylation signal (SV40-polyA), and flanking inverted terminal repeat sequences (ITR).

FIG. 3 is a schematic drawing of an exemplary AAV vector for the expression of the human myotubularin 1 (hMTM1) gene (e.g., a pseudotyped AAV vector, such as an AAV vector containing AAV2 inverted terminal repeats packaged within capsid proteins from AAV8). From left to right, the shaded arrows and rectangles represent the nucleic acid sequences encoding a muscle-specific promoter (e.g., MCK, desmin) operatively linked to hMTM1 and a poly-adenylation signal (polyA), a transcription pause site, a liver-specific promoter (e.g., ApoE/A1AT, LP1) operatively linked to hMTM1, a poly-adenylation signal (polyA), and flanking inverted terminal repeat sequences (ITR).

FIG. 4 is a schematic drawing of an exemplary AAV vector for the expression of the human myotubularin 1 (hMTM1) gene (e.g., a pseudotyped AAV vector, such as an AAV vector containing AAV2 inverted terminal repeats packaged within capsid proteins from AAV8). From left to right, the shaded arrows and rectangles represent the nucleic acid sequences encoding a ubiquitous promoter (e.g., PGK, Ef1a, GAPDH) operatively linked to hMTM1, a poly-adenylation signal (polyA), and flanking inverted terminal repeat sequences (ITR).

FIG. 5 is a graph showing body weight (g) over a period (age in weeks), mean±SEM (n=10-14, for all), measured in the manner described in Example 1, below. Statistical significances: HEMI AAV8-Des-mMTM1 mice vs. WT Vehicle mice: p<0.001 for all time points; WT AAV8-Des-mMTM1+AAV8-Des-hMTM1-STOP mice vs. WT Vehicle mice: p<0.05 for weeks 7-16; HEMI AAV8-Des-mMTM1+AAV8 empty capsid mice vs. HEMI AAV8-Des-mMTM1 mice: p<0.05 on weeks 4 and 5; HEMI AAV8-Des-mMTM1+AAV8-APoE-A1 AT-mMTM1+AAV8-Des-hMTM1-STOP mice vs. HEMI AAV8-Des-mMTM1 mice: p<0.05 on weeks 5-6 and 8-9. FIG. 5 abbreviations: WT, wild type; HEMI, hemizygous.

FIG. 6A is a graph showing alkaline phosphatase (AFOS) (U/L) levels, mean±SEM (n=10-14, for all), measured in the manner described in Example 1, below. No statistical differences were observed. FIG. 6A abbreviations: WT, wild type; HEMI, hemizygous; AFOS, Alkaline Phosphatase.

FIG. 6B is a graph showing alkaline phosphatase (AFOS) (U/L) levels in naïve mice, mean±SEM (n=11), measured in the manner described in Example 1, below. FIG. 6B abbreviations: AFOS, Alkaline Phosphatase.

FIG. 7A is a graph showing alanine aminotransferase (ALAT) (U/L) levels, mean±SEM (in-life samples (weeks 4-10): n=0-7; terminal samples (week 16): n=10-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1 AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Data are not shown for groups that fell below the limit of detection. Statistical differences: *p<0.05 compared to WT Vehicle, #p<0.05 compared to HEMI AAV8-Des-mMTM1. FIG. 7A abbreviations: WT, wild type; HEMI, hemizygous; ALAT, Alanine Aminotransferase.

FIG. 7B is a graph showing alanine aminotransferase (ALAT) (U/L) levels in naïve mice, mean±SEM (n=5-11), measured in the manner described in Example 1, below. FIG. 7B abbreviations: ALAT, Alanine Aminotransferase.

FIG. 8A is a graph showing aspartate aminotransferase (ASAT) (U/L) levels, mean±SEM (n=10-14, for all), measured in the manner described in Example 1, below. Statistical differences: **p<0.01 compared to WT Vehicle. FIG. 8A abbreviations: WT, wild type; HEMI, hemizygous; ASAT, Aspartate Aminotransferase.

FIG. 8B is a graph showing aspartate aminotransferase (ASAT) (U/L) levels in naïve mice, mean±SEM (n=11), measured in the manner described in Example 1, below. FIG. 8B abbreviations: ASAT, Aspartate Aminotransferase.

FIG. 9A is a graph showing alburnin (g/L) levels, mean±SEM (n=10-14, for all), measured in the manner described in Example 1, below. No statistical differences were observed. FIG. 9A abbreviations: WT, wild type; HEMI, hemizygous.

FIG. 9B is a graph showing albumin (g/L) levels in naïve mice, mean±SEM (n=11), measured in the manner described in Example 1, below.

FIG. 10A is a graph showing gamma-glutamyl transferase (GGT) (U/L) levels, mean±SEM (n=10-14, for all), measured in the manner described in Example 1, below. Statistical differences: #p<0.05, ##p<0.01 compared to HEMI AAV8-Des-mMTM1. FIG. 10A abbreviations: WT, wild type; HEMI, hernizygous; GGT, Gamma-Glutamyl Transferase.

FIG. 10B is a graph showing gamma-glutamyl transferase (GGT) (U/L) levels in naïve mice, mean±SEM (n=11), measured in the manner described in Example 1, below. FIG. 10B abbreviations: GGT, Gamma-Glutamyl Transferase.

FIG. 11A is a graph showing total protein (g/L) levels, mean±SEM (n=10-14, for all), measured in the manner described in Example 1, below. No statistical differences were observed.

FIG. 11A abbreviations: WT, wild type: HEMI, hemizygous; Prot tot, total protein.

FIG. 11B is a graph showing total protein (g/L) levels in naïve mice, mean±SEM (n=11), measured in the manner described in Example 1, below. FIG. 11B abbreviations: Prot tot, total protein.

FIG. 12A is a graph showing urea (mmol/L) levels, mean±SEM (n=10-14, for all), measured in the manner described in Example 1, below. No statistical differences were observed. FIG. 12A abbreviations: WT, wild type; HEMI, hemizygous.

FIG. 12B is a graph showing urea (mmol/L) levels in naïve mice, mean±SEM (n=11), measured in the manner described in Example 1, below.

FIG. 13A is a graph showing lactate dehydrogenase (LDH) (U/I) levels, mean±SEM (n=10-14, for all), measured in the manner described in Example 1, below. No statistical differences were observed. FIG. 13A abbreviations: WT, wild type; HEMI, hemizygous; LDH, Lactate Dehydrogenase.

FIG. 13B is a graph showing lactate dehydrogenase (LDH) (U/I) levels in naïve mice, mean±SEM (n=11), measured in the manner described in Example 1, below. FIG. 13B abbreviations: LDH, Lactate Dehydrogenase.

FIG. 14A is a graph showing levels of bile acids (μmol/L), mean±SEM (n=0-3, for all), measured in the manner described in Example 1, below. Data are not shown for groups that fell below the limit of detection. Statistical differences: #p<0.05 compared to HEMI AAV8-Des-mMTM1. FIG. 14A abbreviations: WT, wild type; HEMI, hemizygous.

FIG. 14B is a graph showing levels of bile acids (μmol/L) in naïve mice, mean±SEM (n=11), measured in the manner described in Example 1, below.

FIG. 15 is a graph showing total bilirubin (μmol/L) levels, mean±SEM (n=0-8, for all), measured in the manner described in Example 1, below. Data are not shown for groups that fell below the limit of detection. No statistical differences were observed. FIG. 15 abbreviations: WT, wild type; HEMI, hemizygous; BilTot, total bilirubin.

FIG. 16A is a graph showing calculated globulin (g/L) levels, mean±SEM (n=10-14, for all), measured in the manner described in Example 1, below. Statistical differences: *p<0.05 compared to WT Vehicle. FIG. 16A abbreviations: WT, wild type: HEMI, hemizygous.

FIG. 16B is a graph showing calculated globulin (g/L) levels in naïve mice, mean±SEM (n=11), measured in the manner described in Example 1, below.

FIG. 17A is a graph showing calculated albumin/globulin ratio levels, mean±SEM (n=10-14, for all), measured in the manner described in Example 1, below. No statistical differences were observed. FIG. 17A abbreviations: WT, wild type; HEMI, hemizygous.

FIG. 17B is a graph showing calculated albumin/globulin ratio levels in naïve mice, mean±SEM (n=11), measured in the manner described in Example 1, below.

FIG. 18A is a graph showing creatine kinase (U/L) levels, mean±SEM (n=10-14, for all), measured in the manner described in Example 1, below. Statistical differences: *p<0.05 compared to WT Vehicle. FIG. 18A abbreviations: WT, wild type; HEMI, hemizygous: CK, creatine kinase.

FIG. 18B is a graph showing creatine kinase (U/L) levels in naïve mice, mean±SEM (n=11), measured in the manner described in Example 1, below. FIG. 18B abbreviations: CK, creatine kinase.

FIG. 19A is a graph showing creatine (μmol/L) levels, mean±SEM (n=10-14, for all), measured in the manner described in Example 1, below. No statistical differences were observed.

FIG. 19A abbreviations: WT, wild type: HEMI, hemizygous.

FIG. 19B is a graph showing creatinine (μmol/L) levels in naïve mice, mean±SEM (n=11), measured in the manner described in Example 1, below.

FIG. 20A is a graph showing white blood cell counts (WBC) (×10⁹cells/L), mean±SEM (n=5-14, for al), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: *p<0.05 compared to WT Vehicle, #p<0.05 compared to HEMI AAV8-Des-mMTM1. FIG. 20A abbreviations: WT, wild type; HEMI, hemizygous; WBC, white blood cell count.

FIG. 20B is a graph showing white blood cell counts (WBC) (×10⁹cells/L) levels in naïve mice, mean±SEM (n:=3/9), measured in the manner described in Example 1, below. FIG. 20B abbreviations: WBC, white blood cell count,

FIG. 21A is a graph showing red blood cell counts (RBC) (×10¹²cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-rmMTM1+AAV8-Des-hMTM1-STOP, HEMI AAV8-Des-mMTM1+AAV8 empty capsid, HEMI AAV8-Des-rnMTM1+AAV8-APoE-A1 AT-mMTM1, HEMI AAV8-Des-rmMTM1+AAV8-APoE-A1AT-mMTM1+AAV3-Des-hMTM1-STOP, and WT AAV8-Des-mMTM1+AAV8-Des-hMTM11-STOP. No statistical differences were observed. FIG. 21A abbreviations: WT, wild type; HEMI, hemizygous; RBC, red blood cell count.

FIG. 21B is a graph showing red blood cell counts (RBC) (×10¹²cells/L) levels in naïve mice, mean±SEM (n=3/9), measured in the manner described in Example 1, below. FIG. 21B abbreviations: RBC, red blood cell count.

FIG. 22A is a graph showing hemoglobin (g/L) levels, mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-nMTM1+AAV8-Des-hMTM1-STOP, and WT AAV8-Des-mMTM1+AAV8-Des-hMTM1-STOP, No statistical differences were observed. FIG. 22A abbreviations: WT, wild type: HEMI, hemizygous; HGB, hemoglobin.

FIG. 22B is a graph showing hemoglobin (g/L) levels in naïve mice, mean±SEM (n=3/9), measured in the manner described in Example 1, below. FIG. 22B abbreviations: HGB, hemoglobin.

FIG. 23A is a graph showing hematocrit (HCT) (%) levels, mean±SEM (n=5-14, for all) measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1′-STOP. Statistical differences: #p<0.05 compared to HEMI AAV8-Des-mMTM1 FIG. 23A abbreviations: WT, wild type; HEMI, hemizygous; HCT, hematocrit.

FIG. 23B is a graph showing hematocrit (HCT) (%) levels in naïve mice, mean±SEM (n=3/9), measured in the manner described in Example 1, below. FIG. 23B abbreviations: HCT, hematocrit.

FIG. 24A is a graph showing mean corpuscular volume (MCV) (fL) levels, mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1 AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: *p<0.05 compared to WT Vehicle, #p<0.05 compared to HEMI AAV8-Des-mMTM1. FIG. 24A abbreviations: WT, wild type; HEMI, hemizygous; MCV, mean corpuscular volume.

FIG. 24B is a graph showing mean corpuscular volume (MCV) (fL) levels in naïve mice, mean+SEM (n=3/9), measured in the manner described in Example 1, below. FIG. 24B abbreviations: MCV, mean corpuscular volume.

FIG. 25A is a graph showing mean corpuscular hemoglobin (MCH) (pg) levels, mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mnMTM1+AAV8-Des-hMTM1-STOP, HEMI AAV8-Des-mrMTM1+AAV8 empty capsid, HEMI AAV8-Des-rnMTM1+AAV8-APoE-A1 AT-mMTM11, HEMI AAV8-Des-mnMTM1+AAV8-APoE-A1AT-mMTM1+AAV3-Des-hMTM1-STOP, and WT AAV8-Des-mMTM1+AAV8-Des-hMTM1-STOP. No statistical differences were observed. FIG. 25A abbreviations: WT, wild type: HEMI, hemizygous; MCH, mean corpuscular hemoglobin.

FIG. 25B is a graph showing mean corpuscular hemoglobin (MCH) (pg) levels in naïve mice, mean±SEM (n=3/9), measured in the manner described in Example 1, below. FIG. 25B abbreviations: MCH, mean corpuscular hemoglobin.

FIG. 26A is a graph showing mean corpuscular hemoglobin concentration (MCHC) (g/L) levels, mean±SEM (n=5-14, for all), measured in the manner described in Example 1, below. At each time point, 4, 5, 6, 7, 10, and 16 weeks, the following groups were tested from left to right: WT Vehicle, HEMP AAV3-Des-rnMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1 AT-mMTM1+AAV8-Des-hMTM1-STOP, and WT AAV8-Des-mMTM1+AAV8-Des-hMTM1-STOP. Statistical differences: #p<0.05 compared to HEMI AAV8-Des-mMTM1. FIG. 26A abbreviations: WT, wild type: HEMP, hemizygous; MCHC, mean corpuscular hemoglobin concentration.

FIG. 26B is a graph showing mean corpuscular hemoglobin concentration (MCHC) (g/L) levels in naïve mice, mean±SEM (n=3/9), measured in the manner described in Example 1, below. FIG. 26B abbreviations: MCHC, mean corpuscular hemoglobin concentration.

FIG. 27A is a graph showing thrombocyte counts (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: *p<0.05 compared to WT Vehicle. FIG. 27A abbreviations: WT, wild type; HEMI, hemizygous: PLT, platelet or thrombocyte count.

FIG. 27B is a graph showing thrombocyte counts (×10⁹cells/L) in naïve mice, mean±SEM (n=3/9), measured in the manner described in Example 1, below. FIG. 27B abbreviations: PLT, platelet or thrombocyte count.

FIG. 28A is a graph showing relative neutrophil counts (neutrophil (%)), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1-+AAV8-Des-hMTM1-STOP, HEMI AAV8-Des-mMTM1-+AAV8 empty capsid, HEMP AAV8-Des-rnMTM1+AAV8-APoE-A1AT-mMTM1, HEMI AAV8-Des-mMTM1+AAV8-APoE-A1AT-mMTM1+AAV3-Des-hMTM1-STOP, and WT AAV8-Des-mMTM1+AAV8-Des-hMTM1-STOP. No statistical differences were observed. FIG. 28A abbreviations: WT, wild type; HEMI, hemizygous; % NEUT, Neutrophil (%).

FIG. 28B is a graph showing relative neutrophil counts (neutrophil (%)) in naïve mice, mean±SEM (n=3/9), measured in the manner described in Example 1, below. FIG. 28B abbreviations: % NEUT, Neutrophil (%).

FIG. 29A is a graph showing absolute neutrophil counts (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMP AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM11-STOP. No statistical differences were observed. FIG. 29A abbreviations: WT, wild type; HEMI, hemizygous: Abs/Absolute NEUT, absolute neutrophil count.

FIG. 29B is a graph showing absolute neutrophil counts (×10⁹cells/L) in naïve mice, mean±SEM (n=3/9), measured in the manner described in Example 1, below. FIG. 29B abbreviations: Abs/Absolute NEUT, absolute neutrophil count.

FIG. 30A is a graph showing relative lymphocyte counts (Lymphocyte (%)), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM11+AAV8-Des-hMTM11-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: #p<0.05 compared to HEMI AAV8-Des-mMTM1. FIG. 30A abbreviations: WT, wild type; HEMI, hemizygous; % LYM, Lymphocyte (%).

FIG. 30B is a graph showing relative lymphocyte counts (Lymphocyte (%)) in naïve mice, mean±SEM (n=3/9), measured in the manner described in Example 1, below. FIG. 30B abbreviations: % LYM, Lymphocyte (%).

FIG. 31A is a graph showing absolute lymphocyte counts (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1 AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: *p<0.05 compared to WT Vehicle. FIG. 31A abbreviations: WT, wild type; HEMI, hemizygous; Abs/Absolute LYMPHS, absolute lymphocyte count.

FIG. 31B is a graph showing absolute lymphocyte counts (×10⁹cells/L) in naïve mice, mean±SEM (n=3/9), measured in the manner described in Example 1, below. FIG. 31B abbreviations: Abs/Absolute LYMPHS, absolute lymphocyte count.

FIG. 32A is a graph showing relative monocyte counts (Monocyte (%)), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-rnMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-MTM1+AAV3-Des-hMTM1-STOP, and WT AAV8-Des-rnMTM1+AAV8-Des-hMTM1-STOP. Statistical differences: *p<0.05 compared to WT Vehicle. FIG. 32A abbreviations: WT, wild type; HEMI, hemizygous; % MONO, Monocyte (%).

FIG. 32B is a graph showing relative monocyte counts (Monocyte (%)) in naïve mice, mean±SEM (n=3/9), measured in the manner described in Example 1, below. FIG. 32B abbreviations: % MONO, Monocyte (%).

FIG. 33A is a graph showing absolute monocyte counts (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV3-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: *p<0.05 compared to WT Vehicle. FIG. 33A abbreviations: WT, wild type; HEMI, hemizygous: Abs/Absolute MONOS, absolute monocyte count.

FIG. 33B is a graph showing absolute monocyte counts (×10⁹cells/L) in naïve mice, mean±SEM (n=3/9), measured in the manner described in Example 1, below. FIG. 33B abbreviations: Abs/Absolute MONOS, absolute monocyte count.

FIG. 34A is a graph showing relative eosinophil counts (Eosinophil (%)), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: ##p<0.05 compared to HEMI AAV8-Des-mMTM1. FIG. 34A abbreviations: WT, wild type; HEMI, hemizygous; % EOS, Eosinophil (%).

FIG. 34B is a graph showing relative eosinophil counts (Eosinophil (%)) in naïve mice, mean+SEM (n=3/9), measured in the manner described in Example 1, below. FIG. 34B abbreviations: % EOS, Eosinophil (%).

FIG. 35A is a graph showing absolute eosinophil counts (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1 AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Statistical differences: #p<0.05 compared to HEMI AAV8-Des-mMTM1. FIG. 35A abbreviations: WT, wild type; HEMI, hemizygous; Abs/Absolute EOS, absolute eosinophil count.

FIG. 35B is a graph showing absolute eosinophil counts (×10⁹cells/L) in naïve mice, mean±SEM (n=3/9), measured in the manner described in Example 1, below. FIG. 35B abbreviations: Abs/Absolute EOS, absolute eosinophil count.

FIG. 36A is a graph showing relative basophil counts (Basophil (%)), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-rnMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Data are not shown for groups that fell below the limit of detection. Statistical differences: #p<0.05 compared to HEMI AAV8-Des-mMTM1. FIG. 36A abbreviations: WT, wild type; HEMI, hemizygous: % BASO, Basophil (%).

FIG. 36B is a graph showing relative basophil counts (Basophil (%) in naïve mice, mean±SEM (n=3/9), measured in the manner described in Example 1, below. FIG. 36B abbreviations: % BASO, Basophil (%).

FIG. 37A is a graph showing absolute basophil counts (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM11-STOP, HEMI AAV8-Des-mMTM1+AAV8 empty capsid, HEMI AAV8-Des-mMTM1+AAV8-APoE-A1AT-mMTM1, HEMI AAV8-Des-mMT1M1+AAV8-APoE-A1AT-mMTM1+AAV8-Des-hMTM1-STOP, and WT AAV8-Des-mMTM1+AAV8-Des-hMTM1′-STOP. No statistical differences were observed. Data are not shown for groups that fell below the limit of detection. FIG. 37A abbreviations: WT, wild type: HEMI, hemizygous; Abs/Absolute BASOS, absolute basophil count.

FIG. 37B is a graph showing absolute basophil counts (×10⁹cells/L) in naïve mice, mean±SEM (n=3/9), measured in the manner described in Example 1, below. Data are not shown for groups that fell below the limit of detection. FIG. 37B abbreviations: Abs/Absolute BASOS, absolute basophil count.

FIG. 38A is a graph showing the relative count of large unstained cells (Large unstained cells (%)), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1 AT-mMTM1+AAV8-Des-hMTM1-STOP, and WT AAV8-Des-mMTM1+AAV8-Des-hMTM1-STOP. Statistical differences: **p<0.01 compared to WT Vehicle. FIG. 38A abbreviations: WT, wild type; HEMI, hemizygous; % LUC, Large unstained cells (%).

FIG. 38B is a graph showing the relative count of large unstained cells (Large unstained cells (%)), naïve mice, mean±SEM (n=3/9), measured in the manner described in Example 1, below. FIG. 38B abbreviations: % LUC, Large unstained cells (%).

FIG. 39A is a graph showing the absolute count of large unstained cells (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. Data are not shown for groups that fell below the limit of detection. Statistical differences: #p<0.05, ##p<0.01 compared to HEMI AAV8-Des-mMTM1. FIG. 39A abbreviations: WT, wild type; HEMI, hemizygous; Abs/Absolute LUCS, absolute count of large unstained cells.

FIG. 39B is a graph showing the absolute count of large unstained cells (×10⁹cells/L), naïve mice, mean±SEM (n=3/9), measured in the manner described in Example 1, below. Data are not shown for groups that fell below the limit of detection. FIG. 39B abbreviations: Abs/Absolute LUCS, absolute count of large unstained cells.

FIG. 40A is a graph showing relative reticulocyte counts (Reticulocyte (%)), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1 AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. No statistical differences were observed. FIG. 40A abbreviations: WT, wild type; HEMI, hemizygous; % Retic, Reticulocyte (%).

FIG. 40B is a graph showing relative reticulocyte counts (Reticulocyte (%)) in naïve mice, mean±SEM (n=3/9), measured in the manner described in Example 1, below. FIG. 40B abbreviations: % Retic, Reticulocyte (%).

FIG. 41A is a graph showing absolute reticulocyte cell counts (×10⁹cells/L), mean±SEM (n=5-14, for all), measured in the manner described in Example 1, below. At each time point, 4, 5, 6, 7, 10, and 16 weeks, the following groups were tested from left to right: WT Vehicle, HEMI AAV8-Des-mMTM1, HEMI AAV8-Des-mMTM1+AAV8-Des-hMTM1-STOP, HEMI AAV8-Des-mMTM1+AAV8 empty capsid, HEMI AAV8-Des-mMTM1+AAV8-APoE-A1 AT-mMTM1, HEMI AAV8-Des-mMTM1+AAV8-APoE-A1AT-mMTM1+AAV8-Des-hMTM1-STOP, and WT AAV8-Des-mMTM1+AAV8-Des-hMTM1-STOP. No statistical differences were observed. FIG. 41A abbreviations: WT, wild type; HEMI, hemizygous; Abs/Absolute Retic, absolute reticulocyte cell count.

FIG. 41B is a graph showing absolute reticulocyte cell counts (×10⁹cells/L) in naïve mice, mean±SEM (n=3/9), measured in the manner described in Example 1, below. FIG. 41B abbreviations: Abs/Absolute Retic, absolute reticulocyte cell count.

### DEFINITIONS

As used herein, the term “about” refers to a value that is within 10% above or below the value being described. For example, “100 pounds” as used in the context of weight described herein includes quantities that are within 10% above or below 100 lbs. Additionally, when used in the context of a list of numerical quantities, it is to be understood that the term “about,” when preceding a list of numerical quantities, applies to each individual quantity recited in the list.

As used herein, the terms “administering,” “administration,” and the like refer to directly giving a patient a therapeutic agent (e.g., a pharmaceutical composition including a viral vector including a nucleic acid sequence encoding a myotubularin 1 (MTM1) gene operably linked to a promoter) by any effective route. Exemplary routes of administration are described herein and include systemic administration routes, such as intravenous injection, as well as routes of administration directly to the central nervous system of the patient, such as by way of intrathecal injection or intracerebroventricular injection, and directly to the liver of the patient, among others.

As used herein, the term “age-adjusted norms” refers to the process of a normalization of data by age, which is a technique that is used to allow populations of subjects to be compared when the age profiles of the populations are different. As used herein, the term “norm” refers to data that does not undergo a normalization by age, as populations of subjects across age profiles are similar.

As used herein, the terms “alanine aminotransferase” and “ALT” refer to a protein whose amino acid sequence comprises or consists of an amino acid sequence of a naturally occurring wild-type ALT protein (e.g., ALT1 and ALT2) as well as proteins whose amino acid sequence comprises or consists of an amino acid sequence of a naturally occurring allelic variants of ALT (GPT or GPT2 e.g., splice variants or allelic variants). Human GPT nucleic acid sequence is provided in NCBI RefSeq Acc. No. NM_005309.2, and an exemplary wild-type ALT1 amino acid sequence is provided in NCBI RefSeq Acc. No. NP_005300.1. Human GPT2 nucleic acid sequence is provided in NCBI RefSeq Acc. No. NM_001142466.2, and an exemplary wild-type ALT2 amino acid sequence is provided in NCBI RefSeq Acc. No. NP_001135938.1.

As used herein, the terms “alkaline phosphatase” and “ASP” refer to a protein whose amino acid sequence comprises or consists of an amino acid sequence of a naturally occurring wild-type ASP protein as well as proteins whose amino acid sequence comprises or consists of an amino acid sequence of a naturally occurring allelic variants of ASP (e.g., splice variants or allelic variants). Human ASP nucleic acid sequence is provided in NCBI RefSeq Acc. No. NM_000478.5, and an exemplary wild-type ASP amino acid sequence is provided in NCBI RefSeq Acc. No. NP_000469.3.

As used herein, the term “anti-cholestatic agent” refers to a substance, such as a small molecule that acts to increase bile formation and/or antagonize the effect of hydrophobic bile acids on biological membranes. The term “antagonize,” as used herein with regard to a protein, refers to a molecule that
decreases signal transduction resulting from the interaction of the protein with one or more of its binding partners. The antagonist may result in a decrease in the binding of the protein to one or more of its binding partners relative to binding of the two proteins in the absence of the antagonist.

As used herein, the terms “aspartate aminotransferase” and “AST” refer to a protein whose amino acid sequence comprises or consists of an amino acid sequence of a naturally occurring wild-type AST protein as well as proteins whose amino acid sequence comprises or consists of an amino acid sequence of a naturally occurring allelic variants of AST (e.g., splice variants or allelic variants). Human AST nucleic acid sequence is provided in NCBI RefSeq Acc. No NM_002079.2, and an exemplary wild-type ASP amino acid sequence is provided in NCBI RefSeq Acc. No. NP_002070.1.

As used herein, the terms “bile acid test” and “serum bile acid test” refer to the procedure in which a pre-prandial (i.e., before eating) blood sample is collected for a baseline, followed by a meal and followed about two hours later by the collection of a postprandial (i.e., after eating) blood sample. Both blood samples are tested for bile acid levels and the pre-prandial sample is used as a reference. As used herein, “bile acid” refers to the steroid acids found predominantly in the bile of mammals and other vertebrates.

As used herein, the terms “Children's Hospital of Philadelphia Infant Test of Neuromuscular Disorders” and “CHOP INTEND” refer to a validated motor outcome measure developed for the evaluation of weak infants, such as those with a disease of skeletal muscle (e.g., X-linked myotubular myopathy (XLMTM)). CHOP INTEND uses a 0-64-point scale where higher scores indicate better motor function. As used herein, the term “motor function score” refers to a score on the 0-64-point scale of the CHOP INTEND (e.g., a scale of >45 on the CHOP INTEND).

As used herein, the term “cholestasis” refers to a condition where bile cannot flow from the liver to the duodenum. The two clinical distinctions are the “obstructive” type of cholestasis where there is a mechanical blockage in the duct system that can occur from a gallstone or malignancy, and “metabolic” types of cholestasis which are disturbances in bile formation that can occur because of genetic defects or acquired as a side effect of many medications. As used herein, the term “bile” refers to the digestive fluid that is secreted by the liver to aid in the digestion of fats.

As used herein, a “combination therapy” means that two (or more) different agents or treatments are administered to a subject as part of a defined treatment regimen for a particular disease or condition (e.g., a neuromuscular disorder). In some embodiments, a “combination therapy” may include a procedure. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, intrahepatic routes, and direct absorption through mucous membrane tissues. Therapeutic agents can be administered by the same route or by different routes. For example, a first therapeutic agent of the combination may be administered by intravenous injection while a second therapeutic agent of the combination may be administered enterally. In another example, an agent of the therapeutic combination may be administered by intravenous injection and a procedure (e.g., nasobiliary drainage (NBD)) of the therapeutic combination may be performed.

As used herein, the term “dose” refers to the quantity of a therapeutic agent, such as a viral vector described herein, that is administered to a subject at a particular instant for the treatment of a disorder, such as to treat or ameliorate one or more symptoms of a neuromuscular disorder described herein (e.g., XLMTM). A therapeutic agent as described herein may be administered in a single dose or in multiple doses over the course of a treatment period, as defined herein. In each case, therapeutic agent may be administered using one or more unit dosage forms of therapeutic agent, a term that refers to a one or more discrete compositions containing a therapeutic agent that collectively constitute a single dose of the agent.

As used herein, the terms “effective amount,” “therapeutically effective amount,” and the like, when used in reference to a therapeutic composition, such as a vector construct described herein, refer to a quantity sufficient to, when administered to the subject, including a mammal, for example a human,
effect beneficial or desired results, such as clinical results. For example, in the context of treating neuromuscular disorders, such as XLMTM, these terms refer to an amount of the composition sufficient to achieve a treatment response as compared to the response obtained without administration of the composition of interest. An “effective amount,” “therapeutically effective amount,” and the like, of a composition, such as a vector construct of the present disclosure, also include an amount that results in a beneficial or desired result in a subject as compared to a control.

As used herein, the terms “gamma-glutamyl transferase” and “GGT” refers to a protein whose amino acid sequence comprises or consists of an amino acid sequence of a naturally occurring wild-type GGT protein as well as proteins whose amino acid sequence comprises or consists of an amino acid sequence of a naturally occurring allelic variants of GGT (GGT1, GGT2, and GGT3 e.g., splice variants or allelic variants). Human GGT1 nucleic acid sequence is provided in NCBI RefSeq Acc. No NM_001288833.1, and an exemplary wild-type GGT1 amino acid sequence is provided in NCBI RefSeq Acc. No. NP_001275762.1.

As used herein, the term “hyperbilirubinemia” refers to a condition in which there is a higher-than-normal level of bilirubin in the blood. As used herein, the term “bilirubin” refers to a compound that occurs in the normal catabolic pathway that breaks down heme in vertebrates. This catabolism is a necessary process in the body's clearance of waste products that arise from the destruction of aged or abnormal red blood cells. As used herein, a “bilirubin test” refers to a measurement of the amount of bilirubin in a patient's blood.

As used herein, the term “level” refers to a level of a protein, as compared to a reference. The reference can be any useful reference, as defined herein. By a “decreased level” and an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, ρg/mL, or ng/mL) or percentage relative to total protein in a sample.

As used herein, the terms “liver function test” and “LFT” refers to a hepatic panel (e.g., a group of blood tests that provide information about the state of a patient's liver). A hepatic panel may include measurement of the level of gamma-glutamyl transferase, the level of alkaline phosphatase, the level of aspartate aminotransferase, the level of alanine aminotransferase, the level of albumin, the level of bilirubin, the prothrombin time, the activated partial thromboplastin time, or a combination thereof.

As used herein, the terms “maximal inspiratory pressure” and “MIP” refer to a variable in mechanical ventilation including the total airway pressure delivered, generally used to overcome both respiratory system compliance as well as airway resistance. In as pressure-controlled mode, the MIP includes the sum of the positive-end expiratory pressure and the “delta pressure.” As used herein, the term “delta pressure” refers to a variable in mechanical ventilation including the difference between the MIP and the positive-end expiratory pressure.

As used herein, the term “mechanical ventilatory support” refers to the medical term for artificial ventilation where mechanical means are used to assist or replace spontaneous breathing. As used herein, the term “invasive mechanical ventilatory support” refers to the medical term for artificial ventilation where air is delivered via a tube that is inserted into a patient's windpipe through the mouth or nose and mechanical means are used to assist or replace spontaneous breathing. As used herein, the term “noninvasive mechanical ventilatory support” refers to mechanical ventilatory support in which air is delivered to a patient through a sealed mask that can be placed over the mouth, nose, or the whole face.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the
promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably
linked to one another with no intervening nucleotides present.

As used herein, the term “pharmaceutical composition” refers to a mixture containing a therapeutic compound to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic
promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein in the context of a promoter, the term “selectively active” refers to a promoter that preferentially drives gene expression in one cell or tissue type as compared to another cell or tissue type. For example, a promoter that is “selectively active” in liver cells may achieve a level of gene expression in a liver cell that is greater than the level of gene expression achieved by the same promoter, under substantially the same conditions, in one or more non-liver cells, for example, from 2-fold to 1,000-fold greater (e.g., 2-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, or 1,000-fold greater expression). Exemplary methods for measuring gene expression are known in the art and described herein.

As used herein, a therapeutic agent is considered to be “provided” to a patient if the patient is directly administered therapeutic agent or if the patient is administered a substance that is processed or metabolized in vivo so as to yield therapeutic agent endogenously. For example, a patient, such as a patient having a neuromuscular disorder described herein, may be provided a nucleic acid molecule encoding a therapeutic protein (e.g., MTM1) by direct administration of the nucleic acid molecule or by administration of a substance (e.g., viral vector or cell) that is processed in vivo so as to yield the desired nucleic acid molecule.

As used herein, the terms “patient” and “subject” refer to an organism that receives treatment for a particular disease or condition as described herein (such as a neuromuscular disorder, e.g., XLMTM). Examples of subjects and patients include mammals, such as humans, receiving treatment for a disease or condition described herein.

By a “reference” is meant any useful reference used to compare protein levels related to cholestasis, hyperbilirubinemia, or one or more symptoms thereof. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having cholestasis, hyperbilirubinemia, or one or more symptoms thereof; a sample from a subject that is diagnosed with cholestasis, hyperbilirubinemia, or one or more symptoms thereof; a sample from a subject that has been treated for cholestasis, hyperbilirubinemia, or one or more symptoms thereof; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having cholestasis, hyperbilirubinemia, or one or more symptoms thereof. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “term age” refers to the age of a patient (e.g., a newborn) born between 37 weeks of gestational age and 42 weeks of gestational age. For example, if the patient was born at 35 weeks of gestational age, the patient is at term age at 14 days old.

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “treat” and “treatment” refer to therapeutic treatment, in which the object is to prevent or slow down (lessen) an undesired physiological change or disorder, such as the progression of a neuromuscular disorder, such as XLMTM, among others. Beneficial or desired clinical results include, but are not limited to, alleviation of symptoms (e.g., stiffness and/or joint contractures), diminishment of extent of disease, stabilized (i.e., not worsening) state of disease, delay or slowing of disease progression, amelioration or palliation of the disease state, and remission (whether partial or total), whether detectable or undetectable. In the context of neuromuscular disorders, such as XLMTM, treatment of a patient may manifest in one or more detectable changes, such as an increase in the concentration of MTM1 protein or nucleic acids (e.g., DNA or RNA, such as mRNA) encoding MTM1, or an increase in MTM1 activity (e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or more. The concentration of MTM1 protein may be determined using protein detection assays known in the art, including ELISA assays described herein. The concentration of MTM1-encoding nucleic acids may be determined using nucleic acid detection assays (e.g., RNA Seq assays) described herein. Additionally, treatment of a patient suffering from a neuromuscular disorder, such as XLMTM, may manifest in improvements in a patient's muscle function (e.g., skeletal muscle function) as well as improvements in muscle coordination. For example, manifestation of an improvement may include increasing diaphragm and/or respiratory muscle progression.

As used herein, the terms “X-linked myotubular myopathy” and “XLMTM” refer to the genetically inherited neuromuscular disorder that is caused by mutations of the MTM1 gene and is characterized by symptoms including mild to profound muscle weakness, hypotonia (diminished muscle tone), feeding difficulties, and/or severe breathing complications. Human MTM1 has NCBI Gene ID NO 4534. An exemplary wild-type human MTM1 nucleic acid sequence is provided in NCBI RefSeq Acc. No. NM_000252.3, and an exemplary wild-type myotubularin 1 amino acid sequence is provided in NCBI RefSeq Acc. No. NP_000243.1.

As used herein, the term “vector” refers to a nucleic acid, e.g., DNA or RNA, that may function as a vehicle for the delivery of a gene of interest into a cell (e.g., a mammalian cell, such as a human cell), such as for purposes of replication and/or expression. Exemplary vectors useful in conjunction with the
compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### DETAILED DESCRIPTION

The present disclosure provides compositions and methods that can be used for treating neuromuscular disorders, particularly X-linked myotubular myopathy (XLMTM). In accordance with the compositions and methods described herein, a patient (e.g., a human patient) having XLMTM may be administered a viral vector, such as an adeno-associated viral (AAV) vector, that contains a transgene encoding myotubularin 1 (MTM1). The AAV vector may be, for example, a pseudotyped AAV vector, such as an AAV vector containing AAV2 inverted terminal repeats packaged within capsid proteins from AAV8 (AAV2/8).

In some embodiments, the MTM1 transgene is operably linked to a transcription regulatory element, such as a promoter that drives gene expression in a liver cell. This aspect of the disclosure is based, at least in part, on the discovery that expressing an MTM1 transgene in liver tissue in XLMTM patients may improve the safety of MTM1 gene therapy. In some embodiments, the promoter contains an LP1 promoter, apolipoprotein E (ApoE) promoter, and/or alpha-1-antitrypsin (A1AT) promoter (e.g., an LP1 promoter, ApoE promoter, and/or A1AT promoter described herein).

In some embodiments, the MTM1 transgene is operably linked to a constitutive transcription regulatory element, such as a phosphoglycerate kinase (PGK) promoter, an elongation factor-1 alpha (EF1 alpha) promoter, a glyceraldehyde 3-phosphate dehydrogenase (GAPDH) promote, a cytomegalovirus (CMV) promoter, or a chicken-β-actin (CBA) promoter. In some embodiments, the viral vector includes two MTM1 transgenes: one under the control of a promoter that is active in liver tissue, and another that is under the control of a promoter that is active in muscle tissue. These features of the disclosure are based, at least in part, on the discovery that simultaneously expressing an MTM1 transgene in a plurality of tissues (e.g., liver tissue and muscle tissue) may improve the safety of MTM1 gene therapy.

Also described herein are methods of treating XLMTM in a patient in need thereof by administering to the patient two compositions: (i) a non-viral composition containing a nucleic acid encoding MTM1 operably linked to a promoter that is active (e.g., selectively active) in liver tissue, and (ii) a viral vector containing an MTM1 transgene operably linked to a muscle-specific promoter. Non-viral compositions useful in conjunction with this aspect of the disclosure include, without limitation, a liposome, vesicle, synthetic vesicle, exosome, synthetic exosome, dendrimer, and nanoparticle. In some embodiments, the nanoparticle is a lipid nanoparticle. Viral vectors useful in conjunction with this aspect of the disclosure include, without limitation, resamirigene bilparvovec.

In some embodiments, the disclosure describes a method of reducing stiffness and/or joint contractures in a human patient diagnosed as having XLMTM by administration of one or more compositions described herein.

In some embodiments, the disclosure describes a method of increasing diaphragm and/or respiratory muscle progression in a human patient diagnosed as having XLMTM by administration of one or more compositions described herein.

In some embodiments, the disclosure describes a method of preventing cholestasis or hyperbilirubinemia in a human patient diagnosed as having XLMTM by administration of one or more compositions described herein.

The sections that follow provide a description of therapeutic agents and parameters for assessing cholestasis, hyperbilirubinemia, or one or more symptoms thereof. The following sections also describe various transduction agents that may be used in conjunction with the compositions and methods of the disclosure.

### X-Linked Myotubular Myopathy

XLMTM is a rare, life-threatening, congenital myopathy caused by a mutation in the MTM1 gene and is characterized in most patients by profound muscle weakness and hypotonia at birth, which results in severe respiratory insufficiency, inability to sit up, stand or walk, and early mortality.

The myopathy associated with XLMTM impairs the development of motor skills such as sitting, standing, and walking. Affected infants may also have difficulties with feeding due to muscle weakness. Individuals with this condition often do not have the muscle strength to breathe on their own and must be supported with mechanical ventilation. Some affected individuals require mechanical ventilation only periodically, such as during sleep, while others require mechanical ventilation continuously. Patients having XLMTM may also have weakness in the muscles that control eye movement (ophthalmoplegia), weakness in other muscles of the face, and absent reflexes (areflexia).

In XLMTM, muscle weakness often disrupts normal bone development and can lead to fragile bones, an abnormal curvature of the spine (scoliosis), and joint deformities (contractures) of the hips and knees. Patients having XLMTM may have a large head with a narrow and elongated face and a high, arched roof of the mouth (palate). Patients may also have liver disease, recurrent ear and respiratory infections, or seizures.

As a consequence of their severe breathing difficulties, patients having XLMTM usually survive only into early childhood; however, some patients with this condition have lived into adulthood. The compositions and methods of the disclosure provide the important medical benefit of being able to prolong the lifetimes of such patients by restoring functional MTM1 expression. Moreover, the compositions and methods described herein can be used to improve patients' quality of life post-treatment (e.g., reducing stiffness and/or joint contractures or increasing diaphragm and/or respiratory muscle progression), as the disclosure provides a series of guidelines that can be used to determine a patient's eligibility for being weaned off of mechanical ventilation.

### Methods of Treatment

In some embodiments, the patient is a newborn (e.g., 0-4 months old), an infant (e.g., 0-5 months old), a toddler (e.g., 6-12 months old), a child aged 1-3 years old, or a child aged 3-5 years old at the time of administration of the viral vector.

In some embodiments, the patient is a newborn (e.g., 0-4 months old) at the time of administration of the viral vector. For example, in some embodiments, the patient is a newborn that is about 0 to about 4 months old (e.g., 0 months old to about 4 months old, 1 month old to about 4 months old, 2 months old to about 4 months old, or 3 months old to about 4 months old). In some embodiments, the patient is 0 months old. In some embodiments, the patient is 1 month old. In some embodiments, the patient is 2 months old. In some embodiments, the patient is 3 months old. In some embodiments, the patient is 4 months old.

In some embodiments, the patient is a newborn (e.g., less than about 4 months old) at the time of administration of the viral vector. For example, in some embodiments, the patient is a newborn that is less than about 4 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is an infant (e.g., 0-5 months old) at the time of administration of the viral vector. For example, in some embodiments, the patient is an infant that is about 0 months old to about 5 months old (e.g., 0 months old to about 5 months old, 1 month old to about 5 months old, 2 months old to about 5 months old, 3 months old to about 5 months old, or 4 months old to about 5 months old). In some embodiments, the patient is 0 months old. In some embodiments, the patient is 1 month old. In some embodiments, the patient is 2 months old. In some embodiments, the patient is 3 months old. In some embodiments, the patient is 4 months old. In some embodiments, the patient is 3 months old. In some embodiments, the patient is 5 months old.

In some embodiments, the patient is an infant (e.g., less than about 5 months old) at the time of administration of the viral vector. For example, in some embodiments, the patient is an infant that is less than about 5 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is a toddler (e.g., 6-12 months old) at the time of administration of the viral vector. For example, in some embodiments, the patient is an infant that is about 6 months old to about 12 months old (e.g., 6 months old to about 12 months old, 7 months old to about 12 months old, 8 months old to about 12 months old, 9 months old to about 12 months old, 10 months old to about 12 months old, or 11 months old to about 12 months old). In some embodiments, the patient is 6 months old. In some embodiments, the patient is 7 months old. In some embodiments, the patient is 8 months old. In some embodiments, the patient is 9 months old. In some embodiments, the patient is 10 months old. In some embodiments, the patient is 11 months old. In some embodiments, the patient is 12 months old.

In some embodiments, the patient is a toddler (e.g., less than about 12 months old) at the time of administration of the viral vector. For example, in some embodiments, the patient is a toddler that is less than about 12 months old. In some embodiments, the patient is less than about 12 months old. In some embodiments, the patient is less than about 11 months old. In some embodiments, the patient is less than about 10 months old. In some embodiments, the patient is less than about 9 months old. In some embodiments, the patient is less than about 8 months old. In some embodiments, the patient is less than about 7 months old. In some embodiments, the patient is less than about 6 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is a child aged 1-3 years old at the time of administration of the viral vector. For example, in some embodiments, the patient is a child that is about 1 year old to about 3 years old (e.g., 1 year old to about 3 years old or 2 years old to about 3 years old). In some embodiments, the patient is 1 year old. In some embodiments, the patient is 2 years old. In some embodiments, the patient is 3 years old.

In some embodiments, the patient is a child (e.g., less than about 3 years old) at the time of administration of the viral vector. For example, in some embodiments, the patient is a child that is less than about 3 years old. In some embodiments, the patient is less than about 3 years old. In some embodiments, the patient is less than about 2 years old. In some embodiments, the patient is less than about 1 year old. In some embodiments, the patient is less than about 12 months old. In some embodiments, the patient is less than about 11 months old. In some embodiments, the patient is less than about 10 months old. In some embodiments, the patient is less than about 9 months old. In some embodiments, the patient is less than about 8 months old. In some embodiments, the patient is less than about 7 months old. In some embodiments, the patient is less than about 6 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is a child aged 3-5 years old at the time of administration of the viral vector. For example, in some embodiments, the patient is a child that is about 3 years old to about 5 years old (e.g., 3 years old to about 5 years old or 4 years old to about 5 years old). In some embodiments, the patient is 3 years old. In some embodiments, the patient is 4 years old. In some embodiments, the patient is 5 years old.

In some embodiments, the patient is a child (e.g., less than about 5 years old) at the time of administration of the viral vector. For example, in some embodiments, the patient is a child that is less than about 5 years old. In some embodiments, the patient is less than about 5 years old. In some embodiments, the patient is less than about 4 years old. In some embodiments, the patient is less than about 3 years old. In some embodiments, the patient is less than about 2 years old. In some embodiments, the patient is less than about 1 year old. In some embodiments, the patient is less than about 12 months old. In some embodiments, the patient is less than about 11 months old. In some embodiments, the patient is less than about 10 months old. In some embodiments, the patient is less than about 9 months old. In some embodiments, the patient is less than about 8 months old. In some embodiments, the patient is less than about 7 months old. In some embodiments, the patient is less than about 6 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the viral vector.

In some embodiments, the patient was born at greater than or equal to 35 weeks of gestational age (e.g., 35 weeks of gestational age, 36 weeks of gestational age, 37 weeks of gestational age, 38 weeks of gestational age, 39 weeks of gestational age, 40 weeks of gestational age, 41 weeks of gestational age, and 42 weeks of gestational age) and is between adjusted term age (e.g., 37 weeks of gestational age or greater) to about 5 years old at the time of administration of the viral vector. For example, if the patient was born at 35 weeks of gestational age, the patient is at term age at 14 days old.

In some embodiments, the patient was born at 35 weeks of gestational age and is between adjusted term age to about 5 years old (e.g., 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the viral vector.

In some embodiments, the patient was born at 36 weeks of gestational age and is between adjusted term age to about 5 years old (e.g., 7 days old to about 5 years old, 8 days old to about 5 years old, 9 days old to about 5 years old, 10 days old to about 5 years old, 11 days old to about 5 years old, 12 days old to about 5 years old, 13 days old to about 5 years old, 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the viral vector.

In some embodiments, the patient was born at 37 weeks of gestational age and is between adjusted term age to about 5 years old (e.g., 1 day old to about 5 years old, 2 days old to about 5 years old, 3 days old to about 5 years old, 4 days old to about 5 years old, 5 days old to about 5 years old, 6 days old to about 5 years old, 7 days old to about 5 years old, 8 days old to about 5 years old, 9 days old to about 5 years old, 10 days old to about 5 years old, 11 days old to about 5 years old, 12 days old to about 5 years old, 13 days old to about 5 years old, 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the viral vector.

In some embodiments, the patient is male.

In some embodiments, the patient is female.

### Cholestasis and Hyperbilirubinemia

Cholestasis is any condition in which the flow of bile acid from the liver is slowed or blocked, while hyperbilirubinemia is a condition in which there is an accumulation of bilirubin in the blood and serum bile acids appear to remain normal. By contrast, cholestatic syndromes are characterized by marked bile acidemia with normal to slightly elevated bilirubin levels.

In some embodiments, the patient is monitored for the development of cholestasis. In some embodiments, the patient is monitored for the development of hyperbilirubinemia. In some embodiments, the patient is monitored for the development of cholestasis, hyperbilirubinemia, or one or more symptoms thereof. In some embodiments, the patient is monitored for the development of cholestasis, hyperbilirubinemia, or one or more symptoms thereof by evaluating a parameter in a blood sample obtained from the patient, wherein a finding that the parameter is above a reference level identifies the patient as having cholestasis, hyperbilirubinemia, or one or more symptoms thereof.

In some embodiments, a patient is determined to exhibit cholestasis or one or more symptoms thereof when the patient exhibits one or more parameters (e.g., total bile acids level, gamma-glutamyl transferase (GGT) level, alkaline phosphatase (ASP) level, aspartate aminotransferase (AST) level, and/or alanine aminotransferase (ALT) level), as measured in a serum bile acid test and/or blood test (e.g., a liver function test (LFT)), that is greater than or less than the age-adjusted norm.

In some embodiments, a patient is determined to exhibit hyperbilirubinemia or one or more symptoms thereof when the patient exhibits a bilirubin level, as measured in a blood test (e.g., a bilirubin test), that is greater than the norm.

In some embodiments, the patient does not have a history of cholestasis or hyperbilirubinemia. In some embodiments, the patient does not have a history of any underlying liver disease.

### Vectors for Delivery of Exogenous Nucleic Acids to Target Cells

### Viral Vectors for Nucleic Acid Delivery

Recombinant viral genomes provide a rich source of vectors that can be used for the efficient delivery of a gene of interest (e.g., a transgene encoding MTM1) into the genome of a target cell (e.g., a mammalian cell, such as a human cell). Recombinant viral genomes are particularly useful vectors for gene delivery because they deliver the gene of interest to the nucleus of the target cells. For select viruses, polynucleotides contained within such genomes may be incorporated into the genome of a target cell by generalized or specialized transduction. These processes occur as part of the natural viral replication cycle, and do not require added proteins or reagents. Examples of recombinant viral vectors used to deliver genes of interest include AAV, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the invention include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus
(Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency
virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the invention are recombinant nucleic acid constructs that include (1) a transgene to be expressed (e.g., a polynucleotide encoding a MTM1 protein) and (2) viral nucleic acids that facilitate integration and expression of the heterologous genes. The viral nucleic acids may include those sequences of AAV that are required in cis for replication and packaging (e.g., functional inverted terminal repeats (ITRs)) of the DNA into a virion. In typical applications, the transgene encodes MTM1, which is useful for correcting a MTM1 mutation in patients suffering from neuromuscular disorders, such as XLMTM. Such rAAV vectors may also contain marker or reporter genes. Useful rAAV vectors have one or more of the AAV wild type genes deleted in whole or in part but retain functional flanking ITR sequences. The AAV ITRs may be of any serotype (e.g., derived from serotype 2) suitable for a particular application. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The
construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery.
   rAAV virions useful in conjunction with the compositions and methods described herein include those derived from a variety of AAV serotypes including AAV 1, 2, 3, 4, 5, 6, 7, 8 and 9. For targeting muscle cells, rAAV virions that include at least one serotype 1 capsid protein may be particularly useful. rAAV virions that include at least one serotype 6 capsid protein may also be particularly useful, as serotype 6 capsid proteins are structurally similar to serotype 1 capsid proteins, and thus are expected to also result in high expression of MTM1 in muscle cells. rAAV serotype 9 has also been found to be an efficient transducer of muscle cells. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by
reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8
vector encoding a therapeutic protein pseudotyped with a capsid gene derived from AAV serotype 2. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec.
Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and
epitope tag mutants are described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Resamirigene Bilparvovec

As described herein, a pseudotyped AAV vector including a nucleic acid sequence encoding a MTM1 gene operably linked to a desmin promoter flanked by AAV2 ITR and packaged within capsid proteins from AAV8 (AAV2/8) as well as the other genetic components listed in Table 1, refers to the compound known by the international proprietary name (INN) of resamirigene bilparvovec.

Resamirigene bilparvovec is a non-replicating recombinant AAV8 vector expressing a non-codon-optimized human MTM1 cDNA under the control of the muscle-specific human desmin promoter. The MTM1 expression cassette was built by cloning a synthetic DNA sequence complementary to the coding portion (nucleotides 43-1864) of the wild-type human MTM1 transcript (NCBI Ref. Seq NM_000252.3) downstream of the 1.05-kb human desmin enhancer/promoter region. The second intron and polyadenylation sequence of the human β-globin gene (HBB) were inserted upstream and downstream respectively of the MTM1 synthetic cDNA to mediate RNA processing. The expression cassette was flanked by AAV serotype-2 (AAV2) inverted terminal repeats (ITRs). The vector was produced in an AAV8 capsid by two-plasmid transfection in HEK293 cells in suspension culture in bioreactors with a full GMP process.

In some embodiments, a method of treating a disorder (e.g., XLMTM) or alleviating one or more symptoms (e.g., stiffness and/or join contractures or need for diaphragm and/or respiratory muscle progression) of a disorder (e.g., XLMTM) in a human patient in need thereof, includes administering to the patient a therapeutically effective amount of resamirigene bilparvovec during a treatment period.

In some embodiments, a method of weaning a human patient off of mechanical ventilation, includes a patient that has previously been administered a therapeutically effective amount of resamirigene bilparvovec. The components of resamirigene bilparvovec are shown in Table 1, below:

TABLE 1
Resamirigene Bilparvovec nucleic acid sequence (SEQ ID NO: 1)

| Range | | |
|---|---|---|
| (nucleotides, | | |
| relative to | Length | Genetic |
| SEQ ID NO: 1) | (nucleotides) | Component |
| 3080-3198 | 119 | AAV2 ITR |
| 3199-3256 | 58 | Linker sequence |
| 3257-4316 | 1,060 | Human desmin promoter |
| 4317-4354 | 38 | Linker sequence |
| 4355-4460 | 106 | Human Beta-globin intron |
| 4373-4848 | 476 | Human Beta-globin intron |
| 4458-4902 | 445 | Human Beta-globin intron |
| 4927-6748 | 1,822 | Human MTM1 coding sequence |
| 6749-6759 | 11 | Linker sequence |
| 6760-7519 | 760 | Human Beta-globin poly- |
| | | adenylation sequence |
| 7520-7551 | 32 | Linker sequence |
| 7552 | 7,696 | AAV2 ITR |

As described herein, resamirigene bilparvovec refers to the AAV vector having the nucleic acid sequence of SEQ ID NO: 1, shown below:

TCGCGCGTTT CGGTGATGAC GGTGAAAACC TCTGACACAT GCAGCTCCCG 50
GACGTCATTG TCGATCCTGC AGGCGTACGG TAAAAAAAGG CATAGCTAAC 100
AAGGTGTGGA AAAAGAATTA GTGGTTAGAG AGTGAGCTAT TCGTTGAAAC 150
AATTGCGTTC TTGAAACAAT TCTTGCTGGT AAAATGTCAC ATTTTATGTG 200
ACTACAGGTG GAGGATTGGC ACATAACCTA ACCAGTGGGG GAAACAATTG 250
ACCTCTGGAT TTGTCCAAGT GTATAGTAGC ATTTGCCCAA TCGAATGGTC 300
CTGGTAAGGT GTTAATGTTG ACTAGAACCA AAGGTGGAAG TTGCAGGGAA 350
ACTGGTTTAG TACAAGGGTG GACACCAGGC AGTCATCCAG AGGCCCATTA 400
AAGGCCTTGG AATGTTTTTC CGAAGGAGAA TCACTCCCTC TTCTCTCGCT 450
TAAAGTTTTA GGGGATTCAT GAACAGCTGC TGTGGGATAG TTTCATGTCC 500
CTAGCAATTG TAAAGCAACT GAGGGTGGCT TAAACCAGTT TTAGCTTTAG 550
GGTTAGGGTT ACTGGACTAA AATTTGAGAA ATTCATAAAT CTTAAGGAAA 600
TCCATTGTGA GTTTTCATTA TGAGTGCATC CAATGTATAA TTTCCATGAC 650
CCTCCCATGC AAGTGAGCAT GTGAATCAGG AAACGTTACA AGAACCCAAC 700
AAACTCAACC ACTACTAGAC AGGCGATCAC TTCCAGTTAG TATGCAACTT 750
TCTGTGTAAT TTTAGTTACC ATTAAAATCT GGATGACCTT AGTGTAAGGA 800
AAAAATACCT TGAATAGTGT TAAAGATGTA CACTTGGTGT CAGGCATTGT 850
AACATTGATA AATCTGTGTA AGGTGCTTTT TGAAAACTTC AAAGCTGCAT 900
CAAGTCAAGT ACAAGAAAGG CCATGGCTGC TAAAGCTGTT GAAGATGTGG 950
GATGGAACTG GGTCACATTG GTGTTAACAG CGTTGTGCAG AGCCGGCAGG 1000
ATCTTGGTGT GAGCGAACAT TAGTCTATTT AATAAAGCTG TGTGAATGTT 1050
GTAGAGGTGA GGATGCTCAC TTGAAAACTC ACTGAAGAAC ACTTGGCCCC 1100
TTGAACTAAA GTGCTTCTAT CAAGTTCAGT GAGAAATTCC GAATTACAAG 1150
CATAGGTACT AGAAAAGTTT TGAAAAGCAG TATAGAGCAA CATAAGCACA 1200
TTCATAAAAT TAGTGATGTA GAAAGTGAAA TTTCCACGTA TGGTCACTCC 1250
CAGAGAAAAA AAATACGTTT ATTTACCTTT TTTAAAAATA GGGGATTTCA 1300
GGCCGGGTGA GGTGGCTCAC GCCTGTAATC CCAGCACTTT GGGAGGCCCA 1350
GGTGGGCGGA TCACCTGAGG TCAGGAGTTG GAGGGATGGC AAATCCCATC 1400
TCTACAAAAT ATACAAAAAA ATAGCTGGGT GTGTTGGCAG GCGCCTGTAA 1450
TCCCAGCTAC TCGGAAGGCT GAGGCAGGAG AATCCCTGGA ACCAGGGATG 1500
TGGAGGTTGC AGTGAGCCGA GATTGTGTAA CTGCATTCCA GCCTGGGCAA 1550
CAAGAGCAAG ACTCCGTATC AGGAAAAAAA AAAGGGGGGG TTGGATTTCG 1600
CTTGTTGCAT AGGTTGGTCT CAAACTCCTG GCCTCAAGTG ATTCTCCTGC 1650
CTCTGCCTCC CAAAGTGCTG AGATTACAGG TGTGAGGCAC CATGCCAGGT 1700
CTCTTACTGT TTGTAATTAA ATACATACAC ATTTTGTGTG TTTGTGTGCA 1750
CCTTTATAAA GTCAAAGGTG ATAGTAACCC ATTTAAGTTC CTACTCAATT 1800
TTACTTTCCA GGGATAACTA ACTACTTTTT CTTTTTGAGA TGGAGTCTCG 1850
CTGTGTAGCC CAGGCTGGAG TGCAGTGGCA CCATCTCGGC TCACTGCAAG 1900
CTCCTCCTCC CTGGTTCACG CTATTCTCCT GCCTCAGCCT CCCCAACAAC 1950
TAGGACTACA GGCTCACCTC GCCATACCTG GCTAATTTTT TGTATTTTTA 2000
GTAGAGACAG GGTTTCACTG TGTTAGCCAG GATGGTCTCG ATCTCCTGAC 2050
CTTGTGATCC GCCTGCCTCT GCCTCCCAAA GTGCTGGGAT TACAGGCATG 2100
AGCAACCTCA CCCAGCTGGG ATAACTACTT TTTACAGGTT GATATTCTTT 2150
TGGACTTTTC CCCTGTGTAA AAATATACTA TATTTGTTAT GTACATATTA 2200
TGTACATACA GACACAAATT GGACCATTCT CAGTATAATG ATTCTCAGGT 2250
TTTTTTTTTT TTTTTGAGGT GGGGAACTAG ATAATTATGG ACATCTTTCC 2300
ATACTAGCAT ATCAATATCT ACCTCATTCT TTTTAATATT TTTGCTAGTA 2350
TTCCATTGTA TGAATGTCCT ATGATTTACT TAACCTGTCC ATCAATATTT 2400
GTTTCCAGGT TTTTGCTATT ATAATGCTGC TGCAAAGTAC ATCCTCACAC 2450
ATCTTTATTT TGTCTATTCA TATTTCTGTA AGATAGGTTA CTAAAGTTGG 2500
AACTGCCAAA TTAACACTAT CATACTATTT TGTTTTTTAA TTTTAATTTT 2550
TTAAAAAATG TAAAATGTGC AATTTCAAGA GGAGAAACTT GAACACAAGG 2600
AGCAAAATCT ATTTTTATAA CATCCTATTA AAAGCTTGCT TTACATAAAG 2650
ATTTTGAAAG AATAGCATAA ATACAAGATT TCTATTTTAA TTGGATTCTT 2700
AGGGCTAATA AAATAATCAG CCTTAGCACT TATTTATTTA TTTTTTTTGA 2750
GAGGGAGTCT CGCTCTGTTG TCCATGCTGG AGTGCAGTGG CGTGATCTCG 2800
GCTCACTGCA AGCTCCACCT CATGAGTTCA CACCATTCTC CTGCCTCAGT 2850
CTCCCGAGTA GCTGGGACTC CAGGCGCCCT CTACAAAGCC CGTCTAATTT 2900
TTTTTGTATT TTTAGTAGAG ACAGGGTTTC ACTGTGTTAG CCAGGATGGT 2950
CTTGATCTCC TGACCTTGTG ATCTGCCCGC CTCGGCCTCC CAAAGTGCTG 3000
GGATTATAGG CTTGAGCCAC TGCTCCCGGC CAGCACTTAT TTTTATAATT 3050
CTTCATGATT ACTGTGTTAC TGTCCCATGG GCCGCCAGGG CCAGCTAGGT 3100
TGGCCACTCC CTCTCTGCGC GCTCGCTCGC TCACTGAGGC CGGGCGACCA 3150
AAGGTCGCCC GACGCCCGGG CTTTGCCCGG GCGGCCTCAG TGAGCGAGCG 3200
AGCGCGCAGA GAGGGAGTGG CCAACTCCAT CACTAGGGGT TCCTCCTAGC 3250
ACGCGCTACC CCCTGCCCCC CACAGCTCCT CTCCTGTGCC TTGTTTCCCA 3300
GCCATGCGTT CTCCTCTATA AATACCCGCT CTGGTATTTG GGGTTGGCAG 3350
CTGTTGCTGC CAGGGAGATG GTTGGGTTGA CATGCGGCTC CTGACAAAAC 3400
ACAAACCCCT GGTGTGTGTG GGCGTGGGTG GTGTGAGTAG GGGGATGAAT 3450
CAGGGAGGGG GCGGGGGACC CAGGGGGCAG GAGCCACACA AAGTCTGTGC 3500
GGGGGTGGGA GCGCACATAG CAATTGGAAA CTGAAAGCTT ATCAGACCCT 3550
TTCTGGAAAT CAGCCCACTG TTTATAAACT TGAGGCCCCA CCCTCGACAG 3600
TACCGGGGAG GAAGAGGGCC TGCACTAGTC CAGAGGGAAA CTGAGGCTCA 3650
GGGCCAGCTC GCCCATAGAC ATACATGGCA GGCAGGCTTT GGCCAGGATC 3700
CCTCCGCCTG CCAGGCGTCT CCCTGCCCTC CCTTCCTGCC TAGAGACCCC 3750
CACCCTCAAG CCTGGCTGGT CTTTGCCTGA GACCCAAACC TCTTCGACTT 3800
CAAGAGAATA TTTAGGAACA AGGTGGTTTA GGGCCTTTCC TGGGAACAGG 3850
CCTTGACCCT TTAAGAAATG ACCCAAAGTC TCTCCTTGAC CAAAAAGGGG 3900
ACCCTCAAAC TAAAGGGAAG CCTCTCTTCT GCTGTCTCCC CTGACCCCAC 3950
TCCCCCCCAC CCCAGGACGA GGAGATAACC AGGGCTGAAA GAGGCCCGCC 4000
TGGGGGCTGC AGACATGCTT GCTGCCTGCC CTGGCGAAGG ATTGGTAGGC 4050
TTGCCCGTCA CAGGACCCCC GCTGGCTGAC TCAGGGGCGC AGGCCTCTTG 4100
CGGGGGAGCT GGCCTCCCCG CCCCCACGGC CACGGGCCGC CCTTTCCTGG 4150
CAGGACAGCG GGATCTTGCA GCTGTCAGGG GAGGGGAGGC GGGGGCTGAT 4200
GTCAGGAGGG ATACAAATAG TGCCGACGGC TGGGGGCCCT GTCTCCCCTC 4250
GCCGCATCCA CTCTCCGGCC GGCCGCCTGC CCGCCGCCTC CTCCGTGCGC 4300
CCGCCAGCCT CGCCCGGACT CTAGAGGATC CAGATCTAAG CTTCTCTGGT 4350
CACCGATCCT GAGAACTTCA GGGTGAGTCT ATGGGACCCT TGATGTTTTC 4400
TTTCCCCTTC TTTTCTATGG TTAAGTTCAT GTCATAGGAA GGGGAGAAGT 4450
AACAGGGTAC ACATATTGAC CAAATCAGGG TAATTTTGCA TTTGTAATTT 4500
TAAAAAATGC TTTCTTCTTT TAATATACTT TTTTGTTTAT CTTATTTCTA 4550
ATACTTTCCC TAATCTCTTT CTTTCAGGGC AATAATGATA CAATGTATCA 4600
TGCCTCTTTG CACCATTCTA AAGAATAACA GTGATAATTT CTGGGTTAAG 4650
GCAATAGCAA TATTTCTGCA TATAAATATT TCTGCATATA AATTGTAACT 4700
GATGTAAGAG GTTTCATATT GCTAATAGCA GCTACAATCC AGCTACCATT 4750
CTGCTTTTAT TTTATGGTTG GGATAAGGCT GGATTATTCT GAGTCCAAGC 4800
TAGGCCCTTT TGCTAATCAT GTTCATACCT CTTATCTTCC TCCCACAGCT 4850
CCTGGGCAAC GTGCTGGTCT GTGTGCTGGC CCATCACTTT GGCAAAGAAT 4900
TCCGCGGGCG GCCGCAAGTT TCCAGGATGG CTTCTGCATC AACTTCTAAA 4950
TATAATTCAC ACTCCTTGGA GAATGAGTCT ATTAAGAGGA CGTCTCGAGA 5000
TGGAGTCAAT CGAGATCTCA CTGAGGCTGT TCCTCGACTT CCAGGAGAAA 5050
CACTAATCAC TGACAAAGAA GTTATTTACA TATGTCCTTT CAATGGCCCC 5100
ATTAAGGGAA GAGTTTACAT CACAAATTAT CGTCTTTATT TAAGAAGTTT 5150
GGAAACGGAT TCTTCTCTAA TACTTGATGT TCCTCTGGGT GTGATCTCGA 5200
GAATTGAAAA AATGGGAGGC GCGACAAGTA GAGGAGAAAA TTCCTATGGT 5250
CTAGATATTA CTTGTAAAGA CATGAGAAAC CTGAGGTTCG CTTTGAAACA 5300
GGAAGGCCAC AGCAGAAGAG ATATGTTTGA GATCCTCACG AGATACGCGT 5350
TTCCCCTGGC TCACAGTCTG CCATTATTTG CATTTTTAAA TGAAGAAAAG 5400
TTTAACGTGG ATGGATGGAC AGTTTACAAT CCAGTGGAAG AATACAGGAG 5450
GCAGGGCTTG CCCAATCACC ATTGGAGAAT AACTTTTATT AATAAGTGCT 5500
ATGAGCTCTG TGACACTTAC CCTGCTCTTT TGGTGGTTCC GTATCGTGCC 5550
TCAGATGATG ACCTCCGGAG AGTTGCAACT TTTAGGTCCC GAAATCGAAT 5600
TCCAGTGCTG TCATGGATTC ATCCAGAAAA TAAGACGGTC ATTGTGCGTT 5650
GCAGTCAGCC TCTTGTCGGT ATGAGTGGGA AACGAAATAA AGATGATGAG 5700
AAATATCTCG ATGTTATCAG GGAGACTAAT AAACAAATTT CTAAACTCAC 5750
CATTTATGAT GCAAGACCCA GCGTAAATGC AGTGGCCAAC AAGGCAACAG 5800
GAGGAGGATA TGAAAGTGAT GATGCATATC ATAACGCCGA ACTTTTCTTC 5850
TTAGACATTC ATAATATTCA TGTTATGCGG GAATCTTTAA AAAAAGTGAA 5900
GGACATTGTT TATCCTAATG TAGAAGAATC TCATTGGTTG TCCAGTTTGG 5950
AGTCTACTCA TTGGTTAGAA CATATCAAGC TCGTTTTGAC AGGAGCCATT 6000
CAAGTAGCAG ACAAAGTTTC TTCAGGGAAG AGTTCAGTGC TTGTGCATTG 6050
CAGTGACGGA TGGGACAGGA CTGCTCAGCT GACATCCTTG GCCATGCTGA 6100
TGTTGGATAG CTTCTATAGG AGCATTGAAG GGTTCGAAAT ACTGGTACAA 6150
AAAGAATGGA TAAGTTTTGG ACATAAATTT GCATCTCGAA TAGGTCATGG 6200
TGATAAAAAC CACACCGATG CTGACCGTTC TCCTATTTTT CTCCAGTTTA 6250
TTGATTGTGT GTGGCAAATG TCAAAACAGT TCCCTACAGC TTTTGAATTC 6300
AATGAACAAT TTTTGATTAT AATTTTGGAT CATCTGTATA GTTGCCGATT 6350
TGGTACTTTC TTATTCAACT GTGAATCTGC TCGAGAAAGA CAGAAGGTTA 6400
CAGAAAGGAC TGTTTCTTTA TGGTCACTGA TAAACAGTAA TAAAGAAAAA 6450
TTCAAAAACC CCTTCTATAC TAAAGAAATC AATCGAGTTT TATATCCAGT 6500
TGCCAGTATG CGTCACTTGG AACTCTGGGT GAATTACTAC ATTAGATGGA 6550
ACCCCAGGAT CAAGCAACAA CAGCCGAATC CAGTGGAGCA GCGTTACATG 6600
GAGCTCTTAG CCTTACGCGA CGAATACATA AAGCGGCTTG AGGAACTGCA 6650
GCTCGCCAAC TCTGCCAAGC TTTCTGATCC CCCAACTTCA CCTTCCAGTC 6700
CTTCGCAAAT GATGCCCCAT GTGCAAACTC ACTTCTGACC GGTCCGAGGG 6750
CCCAGATCTA ATTCACCCCA CCAGTGCAGG CTGCCTATCA GAAAGTGGTG 6800
GCTGGTGTGG CTAATGCCCT GGCCCACAAG TATCACTAAG CTCGCTTTCT 6850
TGCTGTCCAA TTTCTATTAA AGGTTCCTTT GTTCCCTAAG TCCAACTACT 6900
AAACTGGGGG ATATTATGAA GGGCCTTGAG CATCTGGATT CTGCCTAATA 6950
AAAAACATTT ATTTTCATTG CAATGATGTA TTTAAATTAT TTCTGAATAT 7000
TTTACTAAAA AGGGAATGTG GGAGGTCAGT GCATTTAAAA CATAAAGAAA 7050
TGAAGAGCTA GTTCAAACCT TGGGAAAATA CACTATATCT TAAACTCCAT 7100
GAAAGAAGGT GAGGCTGCAA ACAGCTAATG CACATTGGCA ACAGCCCCTG 7150
ATGCCTATGC CTTATTCATC CCTCAGAAAA GGATTCAAGT AGAGGCTTGA 7200
TTTGGAGGTT AAAGTTTTGC TATGCTGTAT TTTACATTAC TTATTGTTTT 7250
AGCTGTCCTC ATGAATGTCT TTTCACTACC CATTTGCTTA TCCTGCATCT 7300
CTCAGCCTTG ACTCCACTCA GTTCTCTTGC TTAGAGATAC CACCTTTCCC 7350
CTGAAGTGTT CCTTCCATGT TTTACGGCGA GATGGTTTCT CCTCGCCTGG 7400
CCACTCAGCC TTAGTTGTCT CTGTTGTCTT ATAGAGGTCT ACTTGAAGAA 7450
GGAAAAACAG GGGGCATGGT TTGACTGTCC TGTGAGCCCT TCTTCCCTGC 7500
CTCCCCCACT CACAGTGACC GGCCGCTCTA GGAGGAACCC CTAGTGATGG 7550
AGTTGGCCAC TCCCTCTCTG CGCGCTCGCT CGCTCACTGA GGCCGGGCGA 7600
CCAAAGGTCG CCCGACGCCC GGGCTTTGCC CGGGCGGCCT CAGTGAGCGA 7650
GCGAGCGCGC AGAGAGGGAG TGGCCAACCT AGAGGCCGCC AGGGCCATAT 7700
TTCTCAATTT TTAAATTTTT CAAAAAAATT AATCCTTAAT GTGCATATTT 7750
TTGAATTGTT AATATAACTT TTTGAGGTGA TGTCTTCATG TGTTTCAACT 7800
ACTTAAAAAC TTTTAAACAG TATATAATAA AAAATCTTCC AGGCCACTCA 7850
CACCTGTAAT CCCAGCACTT TGGGAGGCTG AGGTGGGCAG ATCACCTGAG 7900
GGCAGGAGTT CGAGACCAGC CTGGCCAATA TATATATATT CATATATTCA 7950
TATATATATA TATATTCATA TATTCATATA TATATATTCA TATATTCATA 8000
TATATATATA TATATATATA TAGCAAAACC TCATCTCTAA TAAAATACAA 8050
AAATTAGCTG AGCGTGGTGA TGGATGCCTG TAGTCCCAGC TACTCGGGAG 8100
GCTGAGGCAG GAGAATCTCT TGAACCTGGG AGGTGGAGGT TGCAGTGAGC 8150
TGAGATGGTG CCACTGCCCT CCAGCCTGAG TGACAGAGCG AGACTCGGTC 8200
TCCAAAAAAA AACAACAAAA AAATCTTCCA TCCTTGTCTC CCATCCACCC 8250
CTTCCCCCCA GCATGTACTT GCAGACTTTA TGCATATACA GTGAGTACTG 8300
TATATACACA AATAATAAAA AAATCATATA TATAATATAT GTAATTCCCC 8350
TTTACATGAA AGGTAGCACA CTGGTCTGTA CAGTCTGTCT GCACTGTGCT 8400
ATTTCACTTT ATATTTTTAT AGTTTGACAG AGTTCTAACA TTTCTTTTTT 8450
TTTTTTTTTA ACAGAGTCTT GTTCCTGATT GTTAAATTTT AAAGCATCCT 8500
AAAGTTTGGT TTCACACTTG AATGAATACC ATGTAAGGAT TCACTTACAT 8550
AGATGTGGTT GCCTGAATCT TAAGAATAAA ATAACATTGT TTGTATTTAT 8600
TTAAATTAGT GTTCCTTTTA TGGTTTGCCT GAAAGCACAA CAAAATCCTC 8650
ACCAAGATAT TACAATTATG ACTCCCATAC AGGTAAACTG TTTAGAGATT 8700
GGCAAGCACC TTTTAATGAA AGGAGTCAGC CAGCTTAGTG TGCAGTATTT 8750
ATTTCTGCCG GAAGAGGGAG CTTCAGGGAC AGACTTTGGT TTAGTCATGA 8800
AGCCTCCAGC ACTCCCAAGC GGTTGTGGTT GACCAAGCAA TTTATGCTTT 8850
TACCTTTCTA CTTCCAGAGG CTTGTTTACT TATCAGTAAG CATTAATTTA 8900
GTGTCCCCTC AGATGCCTTT TACTTTCTTC TTTTCTGCCT AGAATAAGCT 8950
GCTCTTCCAA TTTTGCAGCT ACATGTTTCC ACCCCAGTTG GAATTTCTCC 9000
ATAACATCCA TTGTAGCTAT CCTTCAATCT ACAGCCTCTA TTTCCTGTTA 9050
TAGCTGGTCA GGTCTAATCC CTCAAAATAC TCTGTCCCCT GCTTCCCTTA 9100
TCTGCTGGCC ACCTTTTTCC CCCACATACA CACTGCCATG TCCCACCCTT 9150
CACTCAAGTT GTTCCCTGCC ACCTCAACAA ATTTAAGTCC ATAAAATAGA 9200
GTAAGTGTTC CTGACTGTTA AATTTTAAAG CATCCCAAAG TCTGATTTCA 9250
CACTCGAATG AATACTATGT ACGGATTCAT TTACATAGAT GCGGTTGCAT 9300
GAGTCTTAAC AAAAAAATAA CATTATTTGT ATTTATTCAA AGTACTGTCA 9350
AGATATAATG TCAAGACCTA ATTCAAAGGT TCCACAAAGC CTTCCTTGAC 9400
TGCCCCCAAC GAAGATTATC CATTTTCCCT GAAATCCCAT TGACTTTTCT 9450
ATTTTGTAAG GAGGCTCGTG AGACTCTGTC TAAAAACAAA ACAAAACAAA 9500
AAGAAACAAT CAAACGGCTT GCTTCTGTTC TTTGATCTGC TAGTAAGCAA 9550
AAATTACACA TGGTGACAGG AGCTATGTGA GGCTGTCAGG TTGAATGGGA 9600
GGAGTTTGGG ATCCTGCTTG TGGATGGTTG GAAGAGGCTT TCGGGAAAGA 9650
CAGTATTTAT GTGAGACCTG GAAGATGGGC CTTAGCTTTG CAGAAGGTGG 9700
AGAGGCAGGA AATAGCACGG GGGCCCTGGG GCTGGAAGAC TTGGGCATAT 9750
TTGAGGAACA GAAAGGAGAC CAGCATAACT GAGGTGGGAA AAGCATGTGA 9800
AGAGATGGGG CTGGAGGAGG CCGGGAGTGG TGGCTCACGC CTGTAATCCC 9850
AGCACTTTGG GAGGCCAAGG CAGGCGGATC ATGAGCTCAG GAGATTGAGA 9900
CCATCCTGGC TAACACGGTG AAACCCCCTC TCTACTAAAA ATACAAAAAA 9950
AAAAAAAAAA AAAATTAGCT GGGCGTGGTG GCAGGAGCCT GTAGTCCCAG 10000
CTACCTGGGA GGCTGAGGCA GGAGAATGGC GTGAACCTGG AAGGCTGAGC 10050
TTGCAGTGAG CCGAGATTGC ACCACTGCAC TCCAGCCTGG GAGACAGAGA 10100
GAGACTCCCT CTCAAAAAAA CAAACAAACG AAACAAAACA AAACAAAAAT 10150
TAGCCAGGCG TGGTGGTATG CACCTGTAAT CCCAGCTACT CGGGAGGTTG 10200
AGGCAGGAGA AACGCTTGAA CTCAGGAGGC GGAGGTTGCA GTGAGCCGAG 10250
ACTGCGCCAC TGCACTCCAG CCTGGGTGAC AGAGGGAGAC TCCATCTCAA 10300
AAAAAAAAAT TTTTTTTTTT TTACAAACGG TGTCTCCCTC TGTCGCCCAG 10350
GCTGGAGTGC AGTGGTGTGA TCACAGCTCA CTCCAGCCTC AACCTCCCCA 10400
GCTGAAGCCA TCCTCTTGCC TCAGCCTCCT AAGTAGCTGG GACTACAGGC 10450
GCGCACCTCC AGGCTTGGCT CTTATTCTTT TTATTGTTTT TGAAACTATA 10500
GAACCTATTT TTAAAAAATG TTTTGGTTGT TTTTATTGCT GCTTTTCCTT 10550
TTGGGGTTAG AACACAAGTT TTGATGGGAA ACAGGTTAGA ACACATTCAT 10600
CTCTTCCCAT AGCGATGGTC ATAGAAAAAC GGGGCATATT TATAAACTCT 10650
CAGTTGATCT TAAAATGTGC AAAAGCTGCC GAACTCCTGG GAGTGAGCTC 10700
GAGCCCTGCA GGATCATTGT CACATGTGAG CAAAAGGCCA GCAAAAGGCC 10750
AGGAACCGTA AAAAGGCCGC GTTGCTGGCG TTTTTCCATA GGCTCCGCCC 10800
CCCTGACGAG CATCACAAAA ATCGACGCTC AAGTCAGAGG TGGCGAAACC 10850
CGACAGGACT ATAAAGATAC CAGGCGTTTC CCCCTGGAAG CTCCCTCGTG 10900
CGCTCTCCTG TTCCGACCCT GCCGCTTACC GGATACCTGT CCGCCTTTCT 10950
CCCTTCGGGA AGCGTGGCGC TTTCTCATAG CTCACGCTGT AGGTATCTCA 11000
GTTCGGTGTA GGTCGTTCGC TCCAAGCTGG GCTGTGTGCA CGAACCCCCC 11050
GTTCAGCCCG ACCGCTGCGC CTTATCCGGT AACTATCGTC TTGAGTCCAA 11100
CCCGGTAAGA CACGACTTAT CGCCACTGGC AGCAGCCACT GGTAACAGGA 11150
TTAGCAGAGC GAGGTATGTA GGCGGTGCTA CAGAGTTCTT GAAGTGGTGG 11200
CCTAACTACG GCTACACTAG AAGAACAGTA TTTGGTATCT GCGCTCTGCT 11250
GAAGCCAGTT ACCTTCGGAA AAAGAGTTGG TAGCTCTTGA TCCGGCAAAC 11300
AAACCACCGC TGGTAGCGGT GGTTTTTTTG TTTGCAAGCA GCAGATTACG 11350
CGCAGAAAAA AAGGATCTCA AGAAGATCCT TTGATCTTTT CTACGGGGTC 11400
TGACGCTCAG TGGAACGAAA ACTCACGTTA AGGGATTTTG GTCATGAGAT 11450
TATCAAAAAG GATCTTCACC TAGATCCTTT TAAATTAAAA ATGAAGTTTT 11500
AAATCAAGCC CAATCTGAAT AATGTTACAA CCAATTAACC AATTCTGATT 11550
AGAAAAACTC ATCGAGCATC AAATGAAACT GCAATTTATT CATATCAGGA 11600
TTATCAATAC CATATTTTTG AAAAAGCCGT TTCTGTAATG AAGGAGAAAA 11650
CTCACCGAGG CAGTTCCATA GGATGGCAAG ATCCTGGTAT CGGTCTGCGA 11700
TTCCGACTCG TCCAACATCA ATACAACCTA TTAATTTCCC CTCGTCAAAA 11750
ATAAGGTTAT CAAGTGAGAA ATCACCATGA GTGACGACTG AATCCGGTGA 11800
GAATGGCAAA AGTTTATGCA TTTCTTTCCA GACTTGTTCA ACAGGCCAGC 11850
CATTACGCTC GTCATCAAAA TCACTCGCAT CAACCAAACC GTTATTCATT 11900
CGTGATTGCG CCTGAGCGAG ACGAAATACG CGATCGCTGT TAAAAGGACA 11950
ATTACAAACA GGAATCGAAT GCAACCGGCG CAGGAACACT GCCAGCGCAT 12000
CAACAATATT TTCACCTGAA TCAGGATATT CTTCTAATAC CTGGAATGCT 12050
GTTTTTCCGG GGATCGCAGT GGTGAGTAAC CATGCATCAT CAGGAGTACG 12100
GATAAAATGC TTGATGGTCG GAAGAGGCAT AAATTCCGTC AGCCAGTTTA 12150
GTCTGACCAT CTCATCTGTA ACATCATTGG CAACGCTACC TTTGCCATGT 12200
TTCAGAAACA ACTCTGGCGC ATCGGGCTTC CCATACAAGC GATAGATTGT 12250
CGCACCTGAT TGCCCGACAT TATCGCGAGC CCATTTATAC CCATATAAAT 12300
CAGCATCCAT GTTGGAATTT AATCGCGGCC TCGACGTTTC CCGTTGAATA 12350
TGGCTCATAA CACCCCTTGT ATTACTGTTT ATGTAAGCAG ACAGTTTTAT 12400
TGTTCATGAT GATATATTTT TATCTTGTGC AATGTAACAT CAGAGATTTT 12450
GAGACACGGG CCAGAGCTGC A 12500

### Transcription Regulatory Elements

Transcription regulatory elements that may be used in conjunction with the compositions and methods described herein may contain various portions operably linked to one another. For example, transcription regulatory elements described herein may contain an ApoE and/or A1AT promoter, such as the chimeric promoter set forth in SEQ ID NO: 2, or a functional portion thereof. Additional nucleic acid regulatory elements useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the above nucleic acid sequence.

Additionally or alternatively, transcription regulatory elements described herein may contain an LP1 promoter, or functional portion thereof. For example, the regulatory element may contain a LP1 promoter as set forth in SEQ ID NO: 3, or a functional portion thereof. Additional nucleic acid regulatory elements useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the above nucleic acid sequences.

Transcription regulatory elements that may be used in conjunction with the compositions and methods described herein include promoters that stimulate expression of a transgene operably linked to a liver-specific promoter. Examples of such promoters are the ApoE/A1At and LP1 promoter, or a variant thereof (e.g., a variant having at least 85% sequence identity (for example, 85%, 86%, 87%, 88%, 89%, 90%, 91%, 92%, 93%, 94%, 95%, 96%, 97%, 98%, 99%, 99.9%, or more, sequence identity) to the nucleic acid sequence of the wild-type promoter locus and that is able to stimulate transcription of a transgene operably linked thereto upon introduction into a cell) or functional portion thereof.

Transcription regulatory elements that may be used in conjunction with the compositions and methods described herein include promoters that stimulate expression of a transgene operably linked to a muscle-specific promoter. Examples of such promoters are the desmin or MCK promoters, or a variant thereof (e.g., a variant having at least 85% sequence identity (for example, 85%, 86%, 87%, 88%, 89%, 90%, 91%, 92%, 93%, 94%, 95%, 96%, 97%, 98%, 99%, 99.9%, or more, sequence identity) to the nucleic acid sequence of the wild-type promoter locus and that is able to stimulate transcription of a transgene operably linked thereto upon introduction into a cell) or functional portion thereof.

Transcription regulatory elements that may be used in conjunction with the compositions and methods described herein include promoters that stimulate expression of a transgene operably linked to a ubiquitous promoter. Examples of such promoters are PGK, Ef1a, and GAPDH or a variant thereof (e.g., a variant having at least 85% sequence identity (for example, 85%, 86%, 87%, 88%, 89%, 90%, 91%, 92%, 93%, 94%, 95%, 96%, 97%, 98%, 99%, 99.9%, or more, sequence identity) to the nucleic acid sequence of the wild-type promoter locus and that is able to stimulate transcription of a transgene operably linked thereto upon introduction into a cell) or functional portion thereof.

Transcription regulatory elements described herein may contain a SV40 enhancer or a functional portion thereof. For example, the regulatory element may contain a SV40 enhancer as set forth in SEQ ID NO: 4, or a functional portion thereof. Additional nucleic acid regulatory elements useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the above nucleic acid sequences.

Transcription regulatory elements described herein may contain a β-globin enhancer or a functional portion thereof. For example, the regulatory element may contain a β-globin enhancer as set forth in SEQ ID NO: 5, or a functional portion thereof. Additional nucleic acid regulatory elements useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the above nucleic acid sequences.

Transcription regulatory elements that may be used in conjunction with the compositions and methods described herein include enhancers that enhance expression of a transgene. Examples of such promoters are PGK, Ff1alpha, and GAPDH, or a variant thereof (e.g., a variant having at least 85% sequence identity (for example, 85%, 86%, 87%, 88%, 89%, 90%, 91%, 92%, 93%, 94%, 95%, 96%, 97%, 98%, 99%, 99.9%, or more, sequence identity) to the nucleic acid sequence of the wild-type promoter locus and that is able to stimulate transcription of a transgene operably linked thereto upon introduction into a cell) or functional portion thereof.

The foregoing nucleic acid regulatory elements are summarized in Table 2, below.

TABLE 2
Exemplary nucleic acid regulatory elements

| SEQ ID | Description of Transcription | |
|---|---|---|
| NO. | Regulatory Element | Nucleic Acid Sequence |
| 2 | ApoE/A1AT promoter (775 nt) | CGTGATCTAGTAGGCTCAGAGGCACACAG |
| | | GAGTTTCTGGGCTCACCCTGCCCCCTTCC |
| | | AACCCCTCAGTTCCCATCCTCCAGCAGCT |
| | | GTTTGTGTGCTGCCTCTGAAGTCCACACT |
| | | GAACAAACTTCAGCCTACTCATGTCCCTA |
| | | AAATGGGCAAACATTGCAAGCAGCAAACA |
| | | GCAAACACACAGCCCTCCCTGCCTGCTGA |
| | | CCTTGGAGCTGGGGCAGAGGTCAGAGAC |
| | | CTCTCTGGGCCCATGCCACCTCCAACATC |
| | | CACTCGACCCCTTGGAATTTCGGTGGAGA |
| | | GGAGCAGAGGTTGTCCTGGCGTGGTTTA |
| | | GGTAGTGTGAGAGGGGTACCCGGGGATC |
| | | TTGCTACCAGTGGAACAGCCACTAAGGAT |
| | | TCTGCAGTGAGAGCAGAGGGCCAGCTAA |
| | | GTGGTACTCTCCCAGAGACTGTCTGACTC |
| | | ACGCCACCCCCTCCACCTTGGACACAGG |
| | | ACGCTGTGGTTTCTGAGCCAGGTACAATG |
| | | ACTCCTTTCGGTAAGTGCAGTGGAAGCTG |
| | | TACACTGCCCAGGCAAAGCGTCCGGGCA |
| | | GCGTAGGGGGGCGACTCAGATCCCAGCC |
| | | AGTGGACTTAGCCCCTGTTTGCTCCTCCG |
| | | ATAACTGGGGTGACCTTGGTTAATATTCA |
| | | CCAGCAGCCTCCCCCGTTGCCCCTCTGG |
| | | ATCCACTGCTTAAATACGGACGAGGACAG |
| | | GGCCCTGTCTCCTCAGCTTCAGGCACCAC |
| | | CACTGACCTGGGACAGTGAATGATCCCCC |
| | | TGATCTGCGGCCTCGACGGTATCGATAAG |
| 3 | LP1 promoter (449 nt) | GTCCCCTAAAATGGGCAAACATTGCAAGC |
| | | AGCAAACAGCAAACACACAGCCCTCCCTG |
| | | CCTGCTGACCTTGGAGCTGGGGCAGAGG |
| | | TCAGAGACCTCTCTGGGCCCATGCCACCT |
| | | CCAACATCCACTCGACCCCTTGGAATTTC |
| | | GGTGGAGAGGAGCAGAGGTTGTCCTGGC |
| | | GTGGTTTAGGTAGTGTGAGAGGGGAATGA |
| | | CTCCTTTCGGTAAGTGCAGTGGAAGCTGT |
| | | ACACTGCCCAGGCAAAGCGTCCGGGCAG |
| | | CGTAGGCGGGCGACTCAGATCCCAGCCA |
| | | GTGcACTTAGCCCCTGTTTGCTCCTCCGA |
| | | TAACTGGGGTGACCTTGGTTAATATTCAC |
| | | CAGCAGCCTCCCCCGTTGCCCCTCTGGAT |
| | | CCACTGOTTAAATACGGACGAGGACAGG |
| | | GCCCTGTCTCCTCAGCTTCAGGCACCACC |
| | | ACTGACCTGGGACAGTGAA |
| 4 | SV40 enhancer (66 nt) | AGGTAAATATAAAATTTTTAAGTGTATAAT |
| | | GTGTTAAACTACTGATTCTAATTGTTTCTC |
| | | TCTTTT |
| 5 | β-globin enhancer (476 nt) | AGGGTGAGTCTATGGGACCCTTGATGTTT |
| | | TCTTTCCCCTTCTTTTCTATGGTTAAGTTC |
| | | ATGTCATAGGAAGGGGAGAAGTAACAGG |
| | | GTACACATATTGACCAAATCAGGGTAATTT |
| | | TGCATTTGTAATTTTAAAAAATGCTTTCTTC |
| | | TTTTAATATACTTTTTTGTTTATCTTATTTCT |
| | | AATACTTTCCCTAATCTCTTTCTTTCAGGG |
| | | CAATAATGATACAATGTATCATGCCTCTTT |
| | | GCACCATTCTAAAGAATAACAGTGATAATT |
| | | TCTGGGTTAAGGCAATAGCAATATTTCTG |
| | | CATATAAATATTTCTGCATATAAATTGTAAC |
| | | TGATGTAAGAGGTTTCATATTGCTAATAGC |
| | | AGCTACAATCCAGCTACCATTCTGCTTTTA |
| | | TTTTATGGTTGGGATAAGGCTGGATTATTC |
| | | TGAGTCCAAGCTAGGCCCTTTTGCTAATC |
| | | ATGTTCATACCTCTTATCTTCCTCCCA |

Additional nucleic acid regulatory elements useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the nucleic acid sequences set forth in Table 2.

### Transgenes

Pseudotyped AAV vectors described herein may include a nucleic acid sequence encoding a MTM1 gene operably linked to a promoter flanked by AAV2 ITR and packaged within capsid proteins from AAV8 (AAV2/8). In some embodiments, the nucleic acid sequence encoding a MTM1 gene encodes a human MTM1 gene. In some embodiments, the nucleic acid sequence encoding a MTM1 gene encodes a mouse MTM1 gene. In some embodiments, the MTM1 gene is codon-optimized. In some embodiments, the promoter is a liver-specific promoter. In some embodiments, the promoter is a muscle-specific promoter. In some embodiments, the promoter is a ubiquitously expressed promoter. In some embodiments two MTM1 genes may be expressed simultaneously using different promoters on the same AAV vector.

Transgenes that may be used in conjunction with the compositions and methods described herein may contain a human MTM1 transgene, as set forth in SEQ ID NO: 6, or a functional portion thereof. Additional transgenes useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the above nucleic acid sequence.

Transgenes that may be used in conjunction with the compositions and methods described herein may contain a mouse MTM1 transgene, as set forth in SEQ ID NO: 7, or a functional portion thereof. Additional transgenes useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the above nucleic acid sequence.

In some embodiments, a method of treating a disorder (e.g., XLMTM) or alleviating one or more symptoms (e.g., stiffness and/or join contractures or need for diaphragm and/or respiratory muscle progression) of a disorder (e.g., XLMTM) in a human patient in need thereof, includes administering to the patient a therapeutically effective amount of a pseudotyped AAV vector including a nucleic acid sequence encoding a MTM1 gene operably linked to a liver-specific or muscle specific promoter flanked by AAV2 ITR and packaged within capsid proteins from AAV8 (AAV2/8) as well as the other genetic components listed in Table 2 during a treatment period.

In some embodiments, a method of weaning a human patient off of mechanical ventilation, includes a patient that has previously been administered a therapeutically effective amount of a pseudotyped AAV vector including a nucleic acid sequence encoding a MTM1 gene operably linked to a liver-specific or muscle specific promoter flanked by AAV2 ITR and packaged within capsid proteins from AAV8 (AAV2/8) as well as the other genetic components listed in Table 2.

The foregoing transgenes are summarized in Table 3, below.

TABLE 3
Exemplary transgenes

| SEQ ID | Description of | |
|---|---|---|
| NO. | Transgene | Nucleic Acid Sequence |
| 6 | Human MTM1 coding | ATGGCTTCTGCATCAACTTCTAAATATAATTCACACTCC |
| | sequence (1812 nt) | TTGGAGAATGAGTCTATTAAGAGGACGTCTCGAGATG |
| | | GAGTCAATCGAGATCTCACTGAGGCTGTTCCTCGACTT |
| | | CCAGGAGAAACACTAATCACTGACAAAGAAGTTATTTA |
| | | CATATGTCCTTTCAATGGCCCCATTAAGGGAAGAGTTT |
| | | ACATCACAAATTATCGTCTTTATTTAAGAAGTTTGGAAA |
| | | CGGATTCTTCTCTAATACTTGATGTTCCTCTGGGTGTG |
| | | ATCTCGAGAATTGAAAAAATGGGAGGCGCGACAAGTA |
| | | GAGGAGAAAATTCCTATGGTCTAGATATTACTTGTAAA |
| | | GACATGAGAAACCTGAGGTTCGCTTTGAAACAGGAAG |
| | | GCCACAGCAGAAGAGATATGTTTGAGATCCTCACGAG |
| | | ATACGCGTTTCCCCTGGCTCACAGTCTGCCATTATTTG |
| | | CATTTTTAAATGAAGAAAAGTTTAACGTGGATGGATGG |
| | | ACAGTTTACAATCCAGTGGAAGAATACAGGAGGCAGG |
| | | GCTTGCCCAATCACCATTGGAGAATAACTTTTATTAATA |
| | | AGTGCTATGAGCTCTGTGACACTTACCCTGCTCTTTTG |
| | | GTGGTTCCGTATCGTGCCTCAGATGATGACCTCCGGA |
| | | GAGTTGCAACTTTTAGGTCCCGAAATCGAATTCCAGTG |
| | | CTGTCATGGATTCATCCAGAAAATAAGACGGTCATTGT |
| | | GCGTTGCAGTCAGCCTCTTGTCGGTATGAGTGGGAAA |
| | | CGAAATAAAGATGATGAGAAATATCTCGATGTTATCAG |
| | | GGAGACTAATAAACAAATTTCTAAACTCACCATTTATGA |
| | | TGCAAGACCCAGCGTAAATGCAGTGGCCAACAAGGCA |
| | | ACAGGAGGAGGATATGAAAGTGATGATGCATATCATAA |
| | | CGCCGAACTTTTCTTCTTAGACATTCATAATATTCATGT |
| | | TATGCGGGAATCTTTAAAAAAAGTGAAGGACATTGTTT |
| | | ATCCTAATGTAGAAGAATCTCATTGGTTGTCCAGTTTG |
| | | GAGTCTACTCATTGGTTAGAACATATCAAGCTCGTTTT |
| | | GACAGGAGCCATTCAAGTAGCAGACAAAGTTTCTTCAG |
| | | GGAAGAGTTCAGTGCTTGTGCATTGCAGTGACGGATG |
| | | GGACAGGACTGCTCAGCTGACATCCTTGGCCATGCTG |
| | | ATGTTGGATAGCTTCTATAGGAGCATTGAAGGGTTCGA |
| | | AATACTGGTACAAAAAGAATGGATAAGTTTTGGACATA |
| | | AATTTGCATCTCGAATAGGTCATGGTGATAAAAACCAC |
| | | ACCGATGCTGACCGTTCTCCTATTTTTCTCCAGTTTATT |
| | | GATTGTGTGTGGCAAATGTCAAAACAGTTCCCTACAGC |
| | | TTTTGAATTCAATGAACAATTTTTGATTATAATTTTGGAT |
| | | CATCTGTATAGTTGCCGATTTGGTACTTTCTTATTCAAC |
| | | TGTGAATCTGCTCGAGAAAGACAGAAGGTTACAGAAA |
| | | GGACTGTTTCTTTATGGTCACTGATAAACAGTAATAAA |
| | | GAAAAATTCAAAAACCCCTTCTATACTAAAGAAATCAAT |
| | | CGAGTTTTATATCCAGTTGCCAGTATGCGTCACTTGGA |
| | | ACTCTGGGTGAATTACTACATTAGATGGAACCCCAGGA |
| | | TCAAGCAACAACAGCCGAATCCAGTGGAGCAGCGTTA |
| | | CATGGAGCTCTTAGCCTTACGCGACGAATACATAAAGC |
| | | GGCTTGAGGAACTGCAGCTCGCCAACTCTGCCAAGCT |
| | | TTCTGATCCCCCAACTTCACCTTCCAGTCCTTCGCAAA |
| | | TGATGCCCCATGTGCAAACTCACTTCTGA |
| 7 | Mouse MTM1 coding | ATGGCTTCTGCATCAGCATCTAAGTATAATTCACACTC |
| | sequence (1812 nt) | CTTGGAGAATGAATCCATTAAGAAAGTGTCTCAAGATG |
| | | GAGTCAGTCAGGATGTGAGTGAGACTGTCCCTCGGCT |
| | | CCCAGGGGAGTTACTAATTACTGAAAAAGAAGTTATTT |
| | | ACATATGTCCTTTCAATGGCCCCATTAAGGGAAGAGTT |
| | | TACATCACAAATTATCGTCTTTATTTAAGAAGTTTGGAA |
| | | ACGGATTCTGCTCTAATACTTGATGTTCCTCTGGGTGT |
| | | GATATCAAGAATTGAAAAAATGGGAGGCGCGACAAGT |
| | | AGAGGAGAAAATTCCTATGGTCTAGATATTACTTGTAA |
| | | AGATTTGAGAAACCTGAGGTTTGCATTGAAGCAAGAAG |
| | | GCCACAGCAGAAGAGATATGTTTGAGATCCTTGTAAAA |
| | | CATGCCTTTCCTCTGGCACACAATCTGCCATTATTTGC |
| | | ATTTGTAAATGAAGAGAAGTTTAACGTGGATGGGTGGA |
| | | CTGTTTATAATCCAGTTGAAGAATATAGAAGGCAGGGC |
| | | CTGCCCAATCACCATTGGAGGATAAGTTTTATTAACAA |
| | | GTGCTATGAGCTCTGTGAGACATACCCTGCTCTTTTGG |
| | | TGGTTCCCTATCGGACCTCAGATGATGATCTTAGGAG |
| | | GATCGCAACGTTTAGATCCCGAAATCGGCTTCCTGTAC |
| | | TGTCGTGGATTCACCCAGAAAACAAAATGGTCATTATG |
| | | CGCTGCAGTCAGCCTCTTGTCGGTATGAGTGGTAAAA |
| | | GAAATAAAGATGACGAGAAATACCTGGATGTGATCAG |
| | | GGAAACTAACAAACAAACTTCTAAGCTCATGATTTATG |
| | | ATGCACGACCCAGTGTAAATGCAGTCGCCAACAAGGC |
| | | AACAGGAGGAGGATATGAAAGTGATGACGCATATCAA |
| | | AACTCAGAACTTTCCTTCTTAGACATTCATAATATTCAT |
| | | GTTATGCGAGAATCTTTAAAAAAAGTGAAAGATATTGTT |
| | | TATCCCAACATAGAAGAATCTCATTGGTTGTCCAGTTT |
| | | GGAGTCTACTCATTGGTTAGAACATATCAAGCTTGTTC |
| | | TGACCGGTGCCATTCAAGTGGCAGACCAAGTGTCTTC |
| | | AGGAAAGAGCTCGGTACTTGTGCACTGCAGTGACGGA |
| | | TGGGACAGGACCGCTCAGCTGACATCCTTGGCCATGC |
| | | TGATGTTGGACAGCTTCTACAGAACTATTGAAGGCTTT |
| | | GAGATATTGGTACAGAAAGAGTGGATAAGTTTTGGCCA |
| | | TAAATTTGCATCTAGAATAGGTCATGGTGATAAAAACC |
| | | ATGCTGATGCTGATCGATCTCCTATTTTTCTTCAGTTTA |
| | | TTGACTGTGTGTGGCAGATGTCGAAACAGTTCCCCAC |
| | | AGCTTTTGAGTTCAATGAAGGCTTTTTGATTACCGTTTT |
| | | GGATCATCTGTATAGCTGTCGATTTGGTACTTTCTTATT |
| | | CAACTGTGACTCGGCTCGAGAAAGACAGAAACTTACA |
| | | GAAAGAACAGTTTCTCTATGGTCGCTAATTAACAGCAA |
| | | TAAAGACAAATTCAAAAACCCCTTCTATACAAAAGAAAT |
| | | CAATCGGGTTTTGTATCCAGTTGCCAGCATGCGTCACT |
| | | TGGAACTGTGGGTGAATTATTACATCCGATGGAATCCC |
| | | AGGGTCAAGCAGCAACAGCCCAACCCAGTGGAGCAG |
| | | CGTTACATGGAGCTTTTGGCCTTGCGTGACGATTATAT |
| | | AAAGAGGCTCGAGGAATTGCAGCTGGCCAACTCCGCC |
| | | AAGCTTGCTGATGCCCCCGCTTCGACTTCCAGTTCGT |
| | | CACAGATGGTGCCCCATGTGCAGACGCACTTCTGA |

Additional transgenes useful in conjunction with the compositions and methods described herein include nucleic acid molecules that have at least 85% sequence identity (e.g., 86%, 87%, 88%, 89%, 90%, 91%, 92%, 93%, 94%, 95%, 96%, 97%, 98%, 99%, 99.9%, or greater, sequence identity) with respect to the nucleic acid sequences set forth in Table 3.

### Methods for the Delivery of Exogenous Nucleic Acids to Target Cells

### Transfection Techniques

Techniques that can be used to introduce a transgene, such as a MTM1 transgene described herein, into a target cell are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids (e.g., nucleic acids capable of expression in e.g., neurons, glial cells, or non-neural cells, such as colon and kidney cells). Electroporation of mammalian cells is described in detail, e.g., in Chu et al., Nucleic Acids Research 15:1311 (1987), the disclosure of which is incorporated herein by reference. A similar technique, NUCLEOFECTION™, utilizes an applied electric field in order to stimulate the uptake of exogenous polynucleotides into the nucleus of a eukaryotic cell. NUCLEOFECTION™ and protocols useful for performing this technique are described in detail, e.g., in Distler et al., Experimental Dermatology 14:315 (2005), as well as in US 2010/0317114, the disclosures of each of which are incorporated herein by reference.

An additional technique useful for the transfection of target cells is the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., J. Vis. Exp. 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Top Curr Chem. 228:227 (2003), the disclosure of which is incorporated herein by reference) polyethylenimine, and DEAE-dextran, the use of which as a transfection agent is described in detail, for example, in Gulick et al., Curr Protoc Mol Biol. 40:1:9.2:9.2.1 (1997), the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

MAGNETOFECTION™ can also be used to deliver nucleic acids to target cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Ther. 9:102 (2002), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US2010/0227406, the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods Cell Biol. 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Target Genes by Gene Editing Techniques

In addition to the above, a variety of tools have been developed that can be used for the incorporation of a gene of interest into a target cell, such as a human cell. One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nat. Rev. Genet. 11:636 (2010); and in Joung et al., Nat. Rev. Mol. Cell Biol. 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Pharmaceutical Compositions and Routes of Administration

The gene therapy agents described herein may contain a transgene, such as a transgene encoding MTM1 and may be incorporated into a vehicle for administration into a patient, such as a human patient suffering from a neuromuscular disorder (for example, XLMTM). Pharmaceutical compositions containing vectors, such as viral vectors, that contain the transcription regulatory elements (e.g., a ApoE/A1AT promoter) described herein operably linked to a therapeutic transgene can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Viral vectors, such as AAV vectors and others described herein, containing the transcription regulatory element operably linked to a therapeutic transgene may be administered to a patient (e.g., a human patient) by a variety of routes of administration. The route of administration may vary, for example, with the onset and severity of disease, and may include, e.g., intradermal, transdermal, parenteral, intravenous, intramuscular, intrahepatic, intranasal, subcutaneous, percutaneous, intratracheal, intraperitoneal, intraarterial, intravascular, inhalation, perfusion, lavage, and oral administration. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, hepatic artery, brachial artery, internal carotid artery, aortic arch, femoral artery, peripheral artery, and/or ciliary artery. It is contemplated that delivery may be through or to an arteriole or capillary.

Mixtures of the nucleic acids and viral vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, intrahepatic, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

### Kits

The compositions described herein can be provided in a kit for use in treating a neuromuscular disorder (e.g., XLMTM). In some embodiments, the kit may include one or more viral vectors as described herein. The kit can include a package insert that instructs a user of the kit, such as a physician of skill in the art, to perform any one of the methods described herein. The kit may optionally include a syringe or other device for administering the composition. In some embodiments, the kit may include one or more additional therapeutic agents.

The kit can include a package insert that instructs a user of the kit, such as a physician of skill in the art, to perform any one of the methods described herein. The kit may optionally include a syringe or other device for administering the composition. In some embodiments, the kit may include one or more additional therapeutic agents.

### Dosing Regimens

### Dosing Regimens Involving AAV-MTM1 Vectors

Using the compositions and methods of the disclosure, a patient having a neuromuscular disorder (e.g., XLMTM) may be administered an AAV vector containing a transgene encoding MTM1 in an amount of about 1.3×10¹⁴ vg/kg. Administration of the vector to the patient in such a quantity can achieve the beneficial effect of augmenting MTM1 expression in the patient, e.g., to within 50% or 200% of wild-type levels, without inducing toxic side effects.

In some embodiments, the AAV vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 2.5×10¹⁴ vg/kg (e.g., in an amount of less than about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 1.5×10¹⁴ vg/kg (e.g., less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 1×10¹⁴ vg/kg (e.g., less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of from about 3×10¹³ vg/kg to about 2.3×10¹⁴ vg/kg (e.g., in an amount of from about 3×10¹³ vg/kg to about 2.3×10¹⁴ vg/kg). For example, the AAV vector may be administered to the patient in an amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 4×10¹³ vg/kg to about 2.3×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 5×10¹³ vg/kg to about 2.3×10¹⁴ vg/kg, such as an in amount of about 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 6×10¹³ vg/kg to about 2.3×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 7×10¹³ vg/kg to about 2.3×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 8×10¹³ vg/kg to about 2.3×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 9×10¹³ vg/kg to about 2.3×10¹⁴ vg/kg, such as an in amount of 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 1×10¹⁴ vg/kg to about 2.3×10¹⁴ vg/kg, such as an in amount 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of about 3×10¹³ vg/kg. In some embodiments, the AAV vector is administered to the patient in an amount of about 4×10¹³ vg/kg. In some embodiments, the AAV vector is administered to the patient in an amount of about 5×10¹³ vg/kg. In some embodiments, the AAV vector is administered to the patient in an amount of about 6×10¹³ vg/kg. In some embodiments, the AAV vector is administered to the patient in an amount of about 7×10¹³ vg/kg. In some embodiments, the AAV vector is administered to the patient in an amount of about 8×10¹³ vg/kg. In some embodiments, the AAV vector is administered to the patient in an amount of about 9×10¹³ vg/kg. In some embodiments, the AAV vector is administered to the patient in an amount of about 1×10¹⁴ vg/kg. In some embodiments, the AAV vector is administered to the patient in an amount of about 1.1×10¹⁴ vg/kg. In some embodiments, the AAV vector is administered to the patient in an amount of about 1.2×10¹⁴ vg/kg. In some embodiments, the AAV vector is administered to the patient in an amount of about 1.3×10¹⁴ vg/kg. In some embodiments, the AAV vector is administered to the patient in an amount of about 1.4×10¹⁴ vg/kg. In some embodiments, the AAV vector is administered to the patient in an amount of about 1.5×10¹⁴ vg/kg. In some embodiments, the AAV vector is administered to the patient in an amount of about 1.6×10¹⁴ vg/kg. In some embodiments, the AAV vector is administered to the patient in an amount of about 1.7×10¹⁴ vg/kg. In some embodiments, the AAV vector is administered to the patient in an amount of about 1.8×10¹⁴ vg/kg. In some embodiments, the AAV vector is administered to the patient in an amount of about 1.9×10¹⁴ vg/kg. In some embodiments, the AAV vector is administered to the patient in an amount of about 2×10¹⁴ vg/kg. In some embodiments, the AAV vector is administered to the patient in an amount of about 2.1×10¹⁴ vg/kg. In some embodiments, the AAV vector is administered to the patient in an amount of about 2.2×10¹⁴ vg/kg. In some embodiments, the AAV vector is administered to the patient in an amount of about 2.3×10¹⁴ vg/kg. In some embodiments, the AAV vector is administered to the patient in an amount of about 2.4×10¹⁴ vg/kg. In some embodiments, the AAV vector is administered to the patient in an amount of about 2.5×10¹⁴ vg/kg. In some embodiments, the AAV vector is administered to the patient in an amount of about 2.6×10¹⁴ vg/kg. In some embodiments, the AAV vector is administered to the patient in an amount of about 2.7×10¹⁴ vg/kg. In some embodiments, the AAV vector is administered to the patient in an amount of about 2.8×10¹⁴ vg/kg. In some embodiments, the AAV vector is administered to the patient in an amount of about 2.9×10¹⁴ vg/kg. In some embodiments, the AAV vector is administered to the patient in an amount of about 3×10¹⁴ vg/kg.

In some embodiments, the AAV vector is administered to the patient in a single dose comprising the amount (e.g., less than about 3×10¹⁴ vg/kg).

In some embodiments, the AAV vector is administered to the patient in two or more (e.g., two, three, four, five, six, seven, eight, nine, or ten) doses that, together, comprise the amount (e.g., less than about 3×10¹⁴ vg/kg).

In some embodiments, the AAV vector is administered to the patient in two or more (e.g., two, three, four, five, six, seven, eight, nine, or ten) doses that each, individually, comprise the amount (e.g., less than about 3×10¹⁴ vg/kg).

In some embodiments, the two or more (e.g., two, three, four, five, six, seven, eight, nine, or ten) doses are separated from one another by one year or more (e.g., one year, one year and one day, one year and one month, one year and six months, two years, three years, four years, or five years).

In some embodiments, the two or more (e.g., two, three, four, five, six, seven, eight, nine, or ten) doses are administered to the patient within about 12 months (e.g., about 12 months, about 11 months, about 10 months, about 9 months, about 8 months, about 7 months, about 6 months, about 5 months, about 4 months, about 3 months, about 2 months, or about 1 month) of one another

### Combination Therapies

An AAV vector containing a transgene encoding MTM1 described herein can be administered in combination with a one or more additional AAV vectors containing a transgene encoding MTM1 described herein or in combination with one or more therapeutic procedures (e.g., nasobiliary drainage (NBD)) and/or agents (e.g., an anti-cholestatic agent) for the treatment of a neuromuscular disorder (e.g., XLMTM).

### Therapeutic Procedure

In some embodiments, the one or more additional therapeutic procedures is NBD. NBD is a therapeutic procedure that is performed to help drain bile (e.g., when the bile duct is blocked, a biliary drain may help bile to flow from the liver into the intestine). In some embodiments, NBD is performed with a biliary drain (also known as a biliary stent), which is a thin, hollow, flexible tube with several small holes along the sides. A biliary drain may be inserted in a patient's bile duct to allow it to drain.

### Therapeutic Agent

In some embodiments, the one or more additional therapeutic agents is an anti-cholestatic agent (e.g., a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor) or a combination thereof.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence within about six weeks before or after (e.g., about six weeks before or after, about five weeks before or after, about four weeks before or after, about three weeks before or after, about two weeks before or after, or about one week before or after) administration of the viral vector to the patient.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence within about five weeks before or after (e.g., about five weeks before or after, about four weeks before or after, about three weeks before or after, about two weeks before or after, or about one week before or after) administration of the viral vector to the patient.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence within about one week before or after (e.g., about one week before or after, about six days before or after, about five days before or after, about four days before or after, about three days before or after, about two days before or after, or about one day before or after) administration of the viral vector to the patient.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence on the same day (e.g., 24^{th} hour, on the 23^{rd} hour, on the 22^{nd} hour, on the 21^{st} hour, on the 20^{th} hour, on the 19^{th} hour, on the 18^{th} hour, on the 17^{th} hour, on the 16^{th} hour, on the 15^{th} hour, on the 14^{th} hour, on the 13^{th} hour, on the 12^{th} hour, on the 11^{th} hour, on the 10^{th} hour, on the 9^{th} hour, on the 8^{th} hour, on the 7^{th} hour, on the 6^{th} hour, on the 5^{th} hour, on the 4^{th} hour, on the 3^{rd} hour, on the 2^{nd} hour, on the 1^{st} hour, on the 60^{th} minute, on the 59^{th} minute, on the 58^{th} minute, on the 57^{th} minute, on the 56^{th} minute, on the 55^{th} minute, on the 50^{th} minute, on the 40^{th} minute, on the 30^{th} minute, on the 20^{th} minute, on the 10^{th} minute, or on the same minute) of administration of the viral vector to the patient.

In some embodiments, the anti-cholestatic agent is a bile acid. In some embodiments, the bile acid is ursodeoxycholic acid or a derivative thereof or nor-ursodeoxycholic acid. In some embodiments, the bile acid is ursodiol.

In some embodiments, the anti-cholestatic agent is an FXR ligand. In some embodiments, the FXR ligand is obeticholic acid, cilofexor, tropifexor, tretinoin, or EDP-305.

In some embodiments, the one or more anti-cholestatic agent is an FGF-19 mimetic. In some embodiment, the FGF-19 mimetic is aldafermin.

In some embodiments, the anti-cholestatic agent is a TGR5 agonist. In some embodiments, the TGR5 agonist is INT-777 or INT-767.

In some embodiments, the anti-cholestatic agent is a PPAR agonist. In some embodiments, the PPAR agonist is bezafibrate, seladelpar, or elafibrinor.

In some embodiments, the anti-cholestatic agent is a PPAR-alpha agonist. In some embodiments, the PPAR-alpha agonist is fenofibrate.

In some embodiments, the anti-cholestatic agent is a PPAR-delta agonist. In some embodiments, the PPAR-delta agonist is seladelpar.

In some embodiments, the anti-cholestatic agent is a dual PPAR-alpha and PPAR-delta agonist. In some embodiments, the dual PPAR-alpha-delta agonist is elafibranor.

In some embodiments, the one or more anti-cholestatic agent is an ASBT inhibitor. In some embodiments, the ASBT inhibitor is odevixibat, maralixibat, or linerixibat.

In some embodiments, the anti-cholestatic agent is an immunomodulatory drug. In some embodiments, the immunomodulatory drug is rituximab, abatacept, ustekinumab, infliximab, baricitinib, or FFP104.

In some embodiments, the anti-cholestatic agent is an antifibrotic therapy. In some embodiments, the antifibrotic therapy is a vitamin D receptor (VDR) agonist or simtuzumab.

In some embodiments, the anti-cholestatic agent is a NOX inhibitor. In some embodiments, the NOX inhibitor is setanaxib.

### Recommended Clinical Parameters for Monitoring a Patient for Development of Cholestasis or Hyperbilirubinemia

In some embodiments, a patient is monitored for the development of cholestasis by a serum bile acid test and/or blood test (e.g., an LFT), as described herein.

In some embodiments, a patient is monitored for the development of hyperbilirubinemia by a blood test (e.g., bilirubin test), as described herein.

In some embodiments, a patient is monitored for the development of cholestasis, and if the patient exhibits cholestasis or one or more symptoms thereof, the patient is administered an anti-cholestatic agent.

In some embodiments, a patient is monitored for the development of hyperbilirubinemia, and if the patient exhibits hyperbilirubinemia or one or more symptoms thereof, the patient is administered an anti-cholestatic agent.

In some embodiments, a patient is monitored for the development of cholestasis or hyperbilirubinemia by a blood test (e.g., a serum acid bile test or a liver function test). In some embodiments, a patient is monitored for the development of cholestasis or hyperbilirubinemia by a blood test (e.g., a serum acid bile test or a liver function test), and if the patient exhibits cholestasis or hyperbilirubinemia or one or more symptoms thereof, the patient is administered an anti-cholestatic agent.

In some embodiments, the patient is determined to exhibit cholestasis, hyperbilirubinemia, or one or more symptoms thereof by a finding that the patient exhibits a parameter (e.g., a serum bile acid level) in blood test (e.g., a serum acid bile test) that is increased relative to a reference level.

In some embodiments, the patient is determined to exhibit cholestasis, hyperbilirubinemia, or one or more symptoms thereof by a finding that the patient exhibits a serum bile acid (e.g., cholic acid, chenodeoxycholic acid, deoxycholic acid, or ursodeoxycholic acid) level in blood test (e.g., a serum acid bile test) that is increased relative to a reference level.

In some embodiments, the blood test is a liver function test.

In some embodiments, a patient is monitored for the development of cholestasis or hyperbilirubinemia by a liver function test, and if the patient exhibits cholestasis or hyperbilirubinemia or one or more symptoms thereof, the patient is administered an anti-cholestatic agent.

In some embodiments, the patient is determined to exhibit cholestasis, hyperbilirubinemia, or one or more symptoms thereof by a finding that the patient exhibits a parameter (e.g., aspartate aminotransferase level or alanine aminotransferase level) in liver function test that is increased or decreased relative to a reference level.

### I. Serum Bile Acid Test

In some embodiments, a patient is monitored for the development of cholestasis or hyperbilirubinemia. In some embodiments, a patient is monitored for the development of cholestasis or hyperbilirubinemia with a serum bile acid test. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia with a serum bile acid test, and if the patient exhibits cholestasis or hyperbilirubinemia or one or more symptoms thereof, the patient is administered an anti-cholestatic agent.

In some embodiments, a patient is monitored for cholestasis or hyperbilirubinemia by the patient's bile acid (e.g., cholic acid, chenodeoxycholic acid, deoxycholic acid, or ursodeoxycholic acid) levels, as measured with a serum bile acid test.

In some embodiments, it is determined that a patient exhibits cholestasis, hyperbilirubinemia, or one or more symptoms thereof and is administered an anti-cholestatic agent when one or more of the patient's bile acid (e.g., cholic acid, chenodeoxycholic acid, deoxycholic acid, or ursodeoxycholic acid) levels, as measured with a serum bile acid test, is greater than the norm.

In some embodiments, it is determined that a patient exhibits cholestasis, hyperbilirubinemia, or one or more symptoms thereof when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's ursodeoxycholic acid level, as measured with a serum bile acid test, is greater than 2 nmol/mL (e.g., 2 nmol/mL, 3 nmol/mL, 4 nmol/mL, 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's ursodeoxycholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

### II. Liver Function Test

In some embodiments, a patient is monitored for the development of cholestasis or hyperbilirubinemia with an LFT. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia with an LFT, and if the patient exhibits cholestasis or hyperbilirubinemia or one or more symptoms thereof, the patient is administered an anti-cholestatic agent.

In some embodiments, it is determined that a patient exhibits cholestasis, hyperbilirubinemia, or one or more symptoms thereof and is administered an anti-cholestatic agent when a parameter (e.g., ASP level or AST level) of the patient's LFT is greater than the age-adjusted norm, as described herein.

IIa. Aspartate Aminotransferase

In some embodiments, a patient is monitored for the development of cholestasis or hyperbilirubinemia by measuring the patient's AST level in an LFT. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia by measuring the patient's AST level in an LFT, and if the patient exhibits cholestasis or hyperbilirubinemia or one or more symptoms thereof, the patient is administered an anti-cholestatic agent.

In some embodiments, a patient is monitored for the development of cholestasis or hyperbilirubinemia by measuring the patient's AST level in an LFT and it is determined that a patient exhibits cholestasis, hyperbilirubinemia, or one or more symptoms thereof and is administered an anti-cholestatic agent when the patient's AST level is greater than the norm.

In some embodiments, it is determined that a patient exhibits cholestasis, hyperbilirubinemia, or one or more symptoms thereof when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis, hyperbilirubinemia, or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIb. Alanine Aminotransferase

In some embodiments, a patient is monitored for the development of cholestasis or hyperbilirubinemia by measuring the patient's ALT level in an LFT. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia by measuring the patient's ALT level in an LFT, and if the patient exhibits cholestasis or hyperbilirubinemia or one or more symptoms thereof, the patient is administered an anti-cholestatic agent.

In some embodiments, a patient is monitored for the development of cholestasis or hyperbilirubinemia by measuring the patient's ALT level in an LFT and it is determined that a patient exhibits cholestasis, hyperbilirubinemia, or one or more symptoms thereof and is administered an anti-cholestatic agent when the patient's ALT level is greater than the norm.

In some embodiments, it is determined that a patient exhibits cholestasis or one or more symptoms thereof when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Recommended Clinical Parameters for Monitoring a Patient for Development of Cholestasis

### I. Serum Bile Acid Test

In some embodiments, a patient is monitored for the development of cholestasis. In some embodiments, a patient is monitored for the development of cholestasis with a serum bile acid test. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis with a serum bile acid test, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when the patient's total bile acids level, as measured with a serum bile acid test, is greater than the norm.

In some embodiments, it is determined that a patient exhibits cholestasis or one or more symptoms thereof and is administered an anti-cholestatic agent when the patient's total bile acids level, as measured with a serum bile acid test, is greater than 14 μmol/L (e.g., 15 μmol/L, 16 μmol/L, 17 μmol/L, 18 μmol/L, 19 μmol/L, 20 μmol/L, 21 μmol/L, 22 μmol/L, 23 μmol/L, 24 μmol/L, 25 μmol/L, 26 μmol/L, 27 μmol/L, 28 μmol/L, 29 μmol/L, 30 μmol/L, 31 μmol/L, 32 μmol/L, 33 μmol/L, 34 μmol/L, 35 μmol/L, 36 μmol/L, 37 μmol/L, 38 μmol/L, 39 μmol/L, 40 μmol/L, 41 μmol/L, 42 μmol/L, 43 μmol/L, 44 μmol/L, 45 μmol/L, 46 μmol/L, 47 μmol/L, 48 μmol/L, 49 μmol/L, 50 μmol/L, 51 μmol/L, 52 μmol/L, 53 μmol/L, 54 μmol/L, 55 μmol/L, 56 μmol/L, 57 μmol/L, 58 μmol/L, 59 μmol/L, 60 μmol/L, 61 μmol/L, 62 μmol/L, 63 μmol/L, 64 μmol/L, 65 μmol/L, 66 μmol/L, 67 μmol/L, 68 μmol/L, 69 μmol/L, 70 μmol/L, 71 μmol/L, 72 μmol/L, 73 μmol/L, 74 μmol/L, 75 μmol/L, 76 μmol/L, 77 μmol/L, 78 μmol/L, 79 μmol/L, 80 μmol/L, 81 μmol/L, 82 μmol/L, 83 μmol/L, 84 μmol/L, 85 μmol/L, 86 μmol/L, 87 μmol/L, 88 μmol/L, 89 μmol/L, 90 μmol/L, 91 μmol/L, 92 μmol/L, 93 μmol/L, 94 μmol/L, 95 μmol/L, 96 μmol/L, 97 μmol/L, 98 μmol/L, 99 μmol/L, and 100 μmol/L).

### II. Blood Test

In some embodiments, a patient is monitored for the development of cholestasis with a blood test (e.g., LFT or a bilirubin test). In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis with an LFT, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when one or more parameters (e.g., GGT level, ASP level, AST level, ALT level, and bilirubin level) of the patient's blood test (e.g., a LFT or a bilirubin test) is greater than the age-adjusted norm, as described herein.

IIa. Liver Function Test

In some embodiments, a patient is monitored for the development of cholestasis with an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis with an LFT, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when one or more parameters (e.g., GGT level, ASP level, AST level, and ALT level) of the patient's LFT is greater than the age-adjusted norm, as described herein.

IIai. Gamma-Glutamyl Transferase

In some embodiments, a patient is monitored for the development of cholestasis by measuring the patient's GGT level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's GGT level in an LFT, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when the patient exhibits a GGT level that is greater than the age-adjusted norm.

In some embodiments, the patient is a newborn (e.g., 0-6 months old), a toddler (e.g., 6-12 months old), or a child aged 1-5 years old. In some embodiments, the patient is a newborn of the age from 0-6 months old. In some embodiments, the patient is a toddler of the age from 6-12 months old. In some embodiments, the patient is a child of the age from 1-5 years old.

In some embodiments, a patient is a newborn (e.g., 0-6 months old) and it is determined that a patient exhibits cholestasis or one or more symptoms thereof and is administered an anti-cholestatic agent when the patient's GGT level is outside of the normal range of about 12-122 U/L (e.g., 12-122 U/L, 13-122 U/L, 14-122 U/L, 15-122 U/L, 16-122 U/L, 17-122 U/L, 18-122 U/L, 19-122 U/L, 20-122 U/L, 25-122 U/L, 30-122 U/L, 40-122 U/L, 50-122 U/L, 60-122 U/L, 70-122 U/L, 80-122 U/L, 90-122 U/L, 100-122 U/L, 110-122 U/L, 120-122 U/L, or 121-122 U/L).

In some embodiments, the patient is a male newborn (e.g., 0-6 months old) and it is determined that a patient exhibits cholestasis or one or more symptoms thereof and is administered an anti-cholestatic agent when the patient's GGT level is less than 12 U/L (e.g., 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a male newborn (e.g., 0-6 months old) and it is determined that a patient exhibits cholestasis or one or more symptoms thereof and is administered an anti-cholestatic agent when the patient's GGT level is greater than 122 U/L (e.g., 123 U/L, 124 U/L, 125 U/L, 126 U/L, 127 U/L, 128 U/L, 129 U/L, 130 U/L, 135 U/L, 140 U/L, 150 U/L, 160 U/L, 170 U/L, 180 U/L, 190 U/L, and 200 U/L).

In some embodiments, the patient is a male toddler (e.g., 6-12 months old) and it is determined that a patient exhibits cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a male toddler (e.g., 6-12 months old) and it is determined that a patient exhibits cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a male child aged 1-5 years old and it is determined that a patient exhibits cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a male child aged 1-5 years old and it is determined that a patient exhibits cholestasis or one or more symptoms thereof and is administered an anti-cholestatic agent when the patient is GGT level is less than 3 U/L (e.g., 2 U/L and 1 U/L).

In some embodiments, the patient is a male child aged 1-5 years old and it is determined that a patient exhibits cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female newborn (e.g., 0-6 months old) and it is determined that a patient exhibits cholestasis or one or more symptoms thereof and is administered an anti-cholestatic agent when the patient's GGT level is outside of the normal range of about 15-132 U/L (e.g., 15-132 U/L, 16-132 U/L, 17-132 U/L, 18-132 U/L, 19-132 U/L, 20-132 U/L, 25-132 U/L, 30-132 U/L, 40-132 U/L, 50-132 U/L, 60-132 U/L, 70-132 U/L, 80-132 U/L, 90-132 U/L, 100-132 U/L, 110-132 U/L, 120-132 U/L, 130-132 U/L, and 131-132 U/L).

In some embodiments, the patient is a female newborn (e.g., 0-6 months old) and it is determined that a patient exhibits cholestasis or one or more symptoms thereof and is administered an anti-cholestatic agent when the patient's GGT level is less than 15 U/L (e.g., 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a female newborn (e.g., 0-6 months old) and it is determined that a patient exhibits cholestasis or one or more symptoms thereof and is administered an anti-cholestatic agent when the patient's GGT level is greater than 132 U/L (e.g., 133 U/L, 134 U/L, 135 U/L, 136 U/L, 137 U/L, 138 U/L, 139 U/L, 140 U/L, 145 U/L, 150 U/L, 160 U/L, 170 U/L, 180 U/L, 190 U/L, and 200 U/L).

In some embodiments, the patient is a female toddler (e.g., 6-12 months old) it is determined that a patient exhibits cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a female toddler (e.g., 6-12 months old) and it is determined that a patient exhibits cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female child aged 1-5 years old it is determined that a patient exhibits cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a female child aged 1-5 years old and it is determined that a patient exhibits cholestasis or one or more symptoms thereof and is administered an anti-cholestatic agent when the patient's GGT level is less than 3 U/L (e.g., 2 U/L and 1 U/L).

In some embodiments, the patient is a female child aged 1-5 years old and it is determined that a patient exhibits cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

IIaii. Alkaline Phosphatase

In some embodiments, a patient is monitored for the development of cholestasis by measuring the patient's ASP level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's ASP level in an LFT, and if the patient exhibits cholestasis or one or more symptoms thereof, the patient is administered an anti-cholestatic agent.

In some embodiments, a patient is monitored for the development of cholestasis by measuring the patient's ASP level in an LFT and it is determined that a patient exhibits cholestasis or one or more symptoms thereof and is administered an anti-cholestatic agent when the patient's ASP level that is greater than the norm.

In some embodiments, it is determined that a patient exhibits cholestasis or one or more symptoms thereof and is administered an anti-cholestatic agent when the patient's ASP level is outside of the normal range of about 50 to 300 U/L e.g., about 51 to 300 U/L, about 52 to U/L, about 53 to 300 U/L, about 54 to 300 U/L, about 55 to 300 U/L, about 56 to 300 U/L, about 57 to 300 U/L, about 58 to 300 U/L, about 59 to 300 U/L, about 60 to 300 U/L, about 65 to 300 U/L, about 70 to 300 U/L, about 80 to 300 U/L, about 90 to 300 U/L, about 100 to 300 U/L, about 125 to 300 U/L, about 150 to 300 U/L, about 175 to 300 U/L, about 200 to 300 U/L, about 225 to 300 U/L, about 250 to 300 U/L, or about 275 to 300 U/L).

In some embodiments, it is determined that a patient exhibits cholestasis or one or more symptoms thereof and is administered an anti-cholestatic agent when the patient's ASP level is less than 50 U/L (e.g., 50 U/L, 49 U/L, 48 U/L, 47 U/L, 46 U/L, 45 U/L, 44 U/L, 43 U/L, 42 U/L, 41 U/L, 40 U/L, 39 U/L, 38 U/L, 37 U/L, 36 U/L, 35 U/L, 34 U/L, 33 U/L, 32 U/L, 31 U/L, 30 U/L, 29 U/L, 28 U/L, 27 U/L, 26 U/L, 25 U/L, 24 U/L, 23 U/L, 22 U/L, 21 U/L, 20 U/L, 19 U/L, 18 U/L, 17 U/L, 16 U/L, 15 U/L, 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, and 1 U/L).

In some embodiments, it is determined that a patient exhibits cholestasis or one or more symptoms thereof and is administered an anti-cholestatic agent when the patient's ASP level is greater than 300 U/L (e.g., 300 U/L, 301 U/L, 302 U/L, 303 U/L, 304 U/L, 305 U/L, 306 U/L, 307 U/L, 308 U/L, 309 U/L, 310 U/L, 311 U/L, 312 U/L, 313 U/L, 314 U/L, 315 U/L, 316 U/L, 317 U/L, 318 U/L, 319 U/L, 320 U/L, 321 U/L, 322 U/L, 323 U/L, 324 U/L, 325 U/L, 330 U/L, 340 U/L, 350 U/L, 400 U/L, and 500 U/L).

IIaiii. Aspartate Aminotransferase

In some embodiments, a patient is monitored for the development of cholestasis by measuring the patient's AST level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's AST level in an LFT, and if the patient exhibits cholestasis or one or more symptoms thereof, the patient is administered an anti-cholestatic agent.

In some embodiments, a patient is monitored for the development of cholestasis by measuring the patient's AST level in an LFT and it is determined that a patient exhibits cholestasis or one or more symptoms thereof and is administered an anti-cholestatic agent when the patient's AST level is greater than the norm.

In some embodiments, it is determined that a patient exhibits cholestasis or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIaiv. Alanine Aminotransferase

In some embodiments, a patient is monitored for the development of cholestasis by measuring the patient's ALT level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's ALT level in an LFT, and if the patient exhibits cholestasis or one or more symptoms thereof, the patient is administered an anti-cholestatic agent.

In some embodiments, a patient is monitored for the development of cholestasis by measuring the patient's ALT level in an LFT and it is determined that a patient exhibits cholestasis or one or more symptoms thereof and is administered an anti-cholestatic agent when the patient's ALT level is greater than the norm.

In some embodiments, it is determined that a patient exhibits cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Recommended Clinical Parameters for Monitoring a Patient for Development of Hyperbilirubinemia

### Bilirubin Test

In some embodiments, a patient is monitored for the development of hyperbilirubinemia. In some embodiments, a patient is monitored for the development of hyperbilirubinemia with a bilirubin test. In some embodiments, a patient is monitored for the development of hyperbilirubinemia, and if the patient exhibits hyperbilirubinemia or one or more symptoms thereof, the patient is administered an anti-cholestatic agent. In some embodiments, a patient is monitored for the development of hyperbilirubinemia with a bilirubin test, and if the patient exhibits hyperbilirubinemia or one or more symptoms thereof, the patient is administered an anti-cholestatic agent.

In some embodiments, it is determined that a patient exhibits hyperbilirubinemia or one or more symptoms thereof and is administered an anti-cholestatic agent when the patient exhibits a bilirubin level that is greater than the norm.

In some embodiments, it is determined that a patient exhibits hyperbilirubinemia or one or more symptoms thereof and is administered an anti-cholestatic agent when the patient's total bilirubin level is greater than 1.2 mg/dL (e.g., 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3 mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, it is determined that a patient exhibits hyperbilirubinemia or one or more symptoms thereof and is administered an anti-cholestatic agent when the patient's direct bilirubin level is greater than 0.2 mg/dL (e.g., 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3 mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, the patient is determined to exhibit hyperbilirubinemia or one or more symptoms thereof and is administered an anti-cholestatic agent when the patient exhibits a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3 mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test.

Recommended Clinical Parameters for Determining that a Patient Exhibits Cholestasis or Hyperbilirubinemia or a Symptom Thereof

In some embodiments, it is determined that the patient exhibits cholestasis, hyperbilirubinemia, or one or more symptoms thereof by determining that one or more parameters (e.g., total bile acids level, GGT level, ASP level, AST level, and ALT level) of the patient's serum bile acid test and/or blood test (e.g., an LFT) is greater than or less than the age-adjusted norm, as described herein, and the patient is administered an anti-cholestatic agent.

In some embodiments, it is determined that the patient exhibits cholestasis, hyperbilirubinemia, or one or more symptoms thereof by determining that one or more parameters (e.g., bilirubin level) of the patient's blood test (e.g., bilirubin test) is greater than the norm, as described herein, and the patient is administered an anti-cholestatic agent.

In some embodiments, it is determined that a patient exhibits cholestasis, hyperbilirubinemia, or one or more symptoms thereof and is administered an anti-cholestatic agent when one or more of the patient's bile acid (e.g., cholic acid, chenodeoxycholic acid, deoxycholic acid, or ursodeoxycholic acid) levels, as measured with a serum bile acid test, is greater than the norm.

In some embodiments, it is determined that a patient exhibits cholestasis, hyperbilirubinemia, or one or more symptoms thereof and is administered an anti-cholestatic agent when a parameter (e.g., ASP level or AST level) of the patient's LFT is greater than the age-adjusted norm, as described herein.

### I. Serum Bile Acid Test

In some embodiments, it is determined that a patient exhibits cholestasis, hyperbilirubinemia, or one or more symptoms thereof and is administered an anti-cholestatic agent when one or more of the patient's bile acid (e.g., cholic acid, chenodeoxycholic acid, deoxycholic acid, or ursodeoxycholic acid) levels, as measured with a serum bile acid test, is greater than the norm.

In some embodiments, it is determined that a patient exhibits cholestasis, hyperbilirubinemia, or one or more symptoms thereof when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's ursodeoxycholic acid level, as measured with a serum bile acid test, is greater than 2 nmol/mL (e.g., 2 nmol/mL, 3 nmol/mL, 4 nmol/mL, 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's ursodeoxycholic acid level, as measured with a serum bile acid test, is greater than 5 nmol/mL (e.g., 2 nmol/mL, 3 nmol/mL, 4 nmol/mL, 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

### II. Liver Function Test

In some embodiments, it is determined that a patient exhibits cholestasis, hyperbilirubinemia, or one or more symptoms thereof and is administered an anti-cholestatic agent when a parameter (e.g., ASP level or AST level) of the patient's LFT is greater than the age-adjusted norm, as described herein.

IIa. Aspartate Aminotransferase

In some embodiments, a patient is monitored for the development of cholestasis or hyperbilirubinemia by measuring the patient's AST level in an LFT and it is determined that a patient exhibits cholestasis, hyperbilirubinemia, or one or more symptoms thereof and is administered an anti-cholestatic agent when the patient's AST level is greater than the norm.

In some embodiments, it is determined that a patient exhibits cholestasis, hyperbilirubinemia, or one or more symptoms thereof when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis, hyperbilirubinemia, or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIb. Alanine Aminotransferase

In some embodiments, a patient is monitored for the development of cholestasis or hyperbilirubinemia by measuring the patient's ALT level in an LFT and it is determined that a patient exhibits cholestasis, hyperbilirubinemia, or one or more symptoms thereof and is administered an anti-cholestatic agent when the patient's ALT level is greater than the norm.

In some embodiments, it is determined that a patient exhibits cholestasis or one or more symptoms thereof when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

Recommended Clinical Parameters for Determining that a Patient Exhibits Cholestasis or a Symptom Thereof

### I. Serum Bile Acid Test

In some embodiments, a patient is determined to exhibit cholestasis or one or more symptoms thereof and is administered an anti-cholestatic agent when the patient exhibits an acid bile level, as measured with a serum bile acid test, that is greater than the norm.

In some embodiments, a patient is determined to exhibit cholestasis or one or more symptoms thereof and is administered an anti-cholestatic agent when the patient's total bile acids level, as measured determined with a serum bile acid test, is greater than 14 μmol/L (e.g., 15 μmol/L, 16 μmol/L, 17 μmol/L, 18 μmol/L, 19 μmol/L, 20 μmol/L, 21 μmol/L, 22 μmol/L, 23 μmol/L, 24 μmol/L, 25 μmol/L, 26 μmol/L, 27 μmol/L, 28 μmol/L, 29 μmol/L, 30 μmol/L, 31 μmol/L, 32 μmol/L, 33 μmol/L, 34 μmol/L, 35 μmol/L, 36 μmol/L, 37 μmol/L, 38 μmol/L, 39 μmol/L, 40 μmol/L, 41 μmol/L, 42 μmol/L, 43 μmol/L, 44 μmol/L, 45 μmol/L, 46 μmol/L, 47 μmol/L, 48 μmol/L, 49 μmol/L, 50 μmol/L, 51 μmol/L, 52 μmol/L, 53 μmol/L, 54 μmol/L, 55 μmol/L, 56 μmol/L, 57 μmol/L, 58 μmol/L, 59 μmol/L, 60 μmol/L, 61 μmol/L, 62 μmol/L, 63 μmol/L, 64 μmol/L, 65 μmol/L, 66 μmol/L, 67 μmol/L, 68 μmol/L, 69 μmol/L, 70 μmol/L, 71 μmol/L, 72 μmol/L, 73 μmol/L, 74 μmol/L, 75 μmol/L, 76 μmol/L, 77 μmol/L, 78 μmol/L, 79 μmol/L, 80 μmol/L, 81 μmol/L, 82 μmol/L, 83 μmol/L, 84 μmol/L, 85 μmol/L, 86 μmol/L, 87 μmol/L, 88 μmol/L, 89 μmol/L, 90 μmol/L, 91 μmol/L, 92 μmol/L, 93 μmol/L, 94 μmol/L, 95 μmol/L, 96 μmol/L, 97 μmol/L, 98 μmol/L, 99 μmol/L, and 100 μmol/L).

### II. Blood Test

In some embodiments, a patient is determined to exhibit cholestasis or one or more symptoms thereof and is administered an anti-cholestatic agent when one or more parameters (e.g., GGT level, ASP level, AST level, ALT level, and bilirubin level) of the patient's blood test (e.g., a LFT or a bilirubin test) is greater than the age-adjusted norm, as described herein.

IIa. Liver Function Test

In some embodiments, a patient is determined to exhibit cholestasis or one or more symptoms thereof and is administered an anti-cholestatic agent when one or more parameters (e.g., GGT level, ASP level, AST level, and ALT level) of the patient's LFT is greater than the age-adjusted norm, as described herein.

IIai. Gamma-Glutamyl Transferase

In some embodiments, a patient is determined to exhibit cholestasis or one or more symptoms thereof and is administered an anti-cholestatic agent when the patient exhibits a GGT level, as measured in a LFT, that is greater than the age-adjusted norm.

In some embodiments, the patient is a newborn (e.g., 0-6 months old), a toddler (e.g., 6-12 months old), or a child aged 1-5 years old. In some embodiments, the patient is a newborn of the age from 0-6 months old. In some embodiments, the patient is a toddler of the age from 6-12 months old. In some embodiments, the patient is a child of the age from 1-5 years old.

In some embodiments, a patient is a newborn (e.g., 0-6 months old) and is determined to exhibit cholestasis or one or more symptoms thereof and is administered an anti-cholestatic agent when the patient's GGT level is outside of the normal range of about 12-122 U/L (e.g., 12-122 U/L, 13-122 U/L, 14-122 U/L, 15-122 U/L, 16-122 U/L, 17-122 U/L, 18-122 U/L, 19-122 U/L, 20-122 U/L, 25-122 U/L, 30-122 U/L, 40-122 U/L, 50-122 U/L, 60-122 U/L, 70-122 U/L, 80-122 U/L, 90-122 U/L, 100-122 U/L, 110-122 U/L, 120-122 U/L, or 121-122 U/L).

In some embodiments, the patient is a male newborn (e.g., 0-6 months old) and is determined to exhibit cholestasis or one or more symptoms thereof and is administered an anti-cholestatic agent when the patient's GGT level is less than 12 U/L (e.g., 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a male newborn (e.g., 0-6 months old) and is determined to exhibit cholestasis or one or more symptoms thereof and is administered an anti-cholestatic agent when the patient's GGT level is greater than 122 U/L (e.g., 123 U/L, 124 U/L, 125 U/L, 126 U/L, 127 U/L, 128 U/L, 129 U/L, 130 U/L, 135 U/L, 140 U/L, 150 U/L, 160 U/L, 170 U/L, 180 U/L, 190 U/L, and 200 U/L).

In some embodiments, the patient is a male toddler (e.g., 6-12 months old) and is determined to exhibit cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a male toddler (e.g., 6-12 months old) and is determined to exhibit cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a male child aged 1-5 years old and is determined to exhibit cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a male child aged 1-5 years old and is determined to exhibit cholestasis or one or more symptoms thereof and is administered an anti-cholestatic agent when the patient's GGT level is less than about 3 U/L (e.g., 2 U/L and 1 U/L).

In some embodiments, the patient is a male child aged 1-5 years old and is determined to exhibit cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female newborn (e.g., 0-6 months old) and is determined to exhibit cholestasis or one or more symptoms thereof and is administered an anti-cholestatic agent when the patient's GGT level is outside of the normal range of about 15-132 U/L (e.g., 15-132 U/L, 16-132 U/L, 17-132 U/L, 18-132 U/L, 19-132 U/L, 20-132 U/L, 25-132 U/L, 30-132 U/L, 40-132 U/L, 50-132 U/L, 60-132 U/L, 70-132 U/L, 80-132 U/L, 90-132 U/L, 100-132 U/L, 110-132 U/L, 120-132 U/L, 130-132 U/L, and 131-132 U/L).

In some embodiments, the patient is a female newborn (e.g., 0-6 months old) and is determined to exhibit cholestasis or one or more symptoms thereof and is administered an anti-cholestatic agent when the patient's GGT level is less than about 15 U/L (e.g., 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a female newborn (e.g., 0-6 months old) and is determined to exhibit cholestasis or one or more symptoms thereof and is administered an anti-cholestatic agent when the patient's GGT level is greater than 132 U/L (e.g., 133 U/L, 134 U/L, 135 U/L, 136 U/L, 137 U/L, 138 U/L, 139 U/L, 140 U/L, 145 U/L, 150 U/L, 160 U/L, 170 U/L, 180 U/L, 190 U/L, and 200 U/L).

In some embodiments, the patient is a female toddler (e.g., 6-12 months old) and is determined to exhibit cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a female toddler (e.g., 6-12 months old) and is determined to exhibit cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female child aged 1-5 years old and is determined to exhibit cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a female child aged 1-5 years old and is determined to exhibit cholestasis or one or more symptoms thereof and is administered an anti-cholestatic agent when the patient's GGT level is less than about 3 U/L (e.g., 2 U/L and 1 U/L).

In some embodiments, the patient is a female child aged 1-5 years old and is determined to exhibit cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

IIaii. Alkaline Phosphatase

In some embodiments, a patient is determined to exhibit cholestasis or one or more symptoms thereof and is administered an anti-cholestatic agent when the patient exhibits an ASP level, as measured in a LFT, that is greater than the norm.

In some embodiments, a patient is determined to exhibit cholestasis or one or more symptoms thereof and is administered an anti-cholestatic agent when the patient's ASP level is outside of the normal range of about 50 to 300 U/L (e.g., about 51 to 300 U/L, about 52 to U/L, about 53 to 300 U/L, about 54 to 300 U/L, about 55 to 300 U/L, about 56 to 300 U/L, about 57 to 300 U/L, about 58 to 300 U/L, about 59 to 300 U/L, about 60 to 300 U/L, about 65 to 300 U/L, about 70 to 300 U/L, about 80 to 300 U/L, about 90 to 300 U/L, about 100 to 300 U/L, about 125 to 300 U/L, about 150 to 300 U/L, about 175 to 300 U/L, about 200 to 300 U/L, about 225 to 300 U/L, about 250 to 300 U/L, or about 275 to 300 U/L).

In some embodiments, a patient is determined to exhibit cholestasis or one or more symptoms thereof and is administered an anti-cholestatic agent when the patient's ASP level is less than about 50 U/L (e.g., 50 U/L, 49 U/L, 48 U/L, 47 U/L, 46 U/L, 45 U/L, 44 U/L, 43 U/L, 42 U/L, 41 U/L, 40 U/L, 39 U/L, 38 U/L, 37 U/L, 36 U/L, 35 U/L, 34 U/L, 33 U/L, 32 U/L, 31 U/L, 30 U/L, 29 U/L, 28 U/L, 27 U/L, 26 U/L, 25 U/L, 24 U/L, 23 U/L, 22 U/L, 21 U/L, 20 U/L, 19 U/L, 18 U/L, 17 U/L, 16 U/L, 15 U/L, 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, and 1 U/L).

In some embodiments, a is determined to exhibit cholestasis or one or more symptoms thereof and is administered an anti-cholestatic agent when the patient's ASP level is greater than 300 U/L (e.g., 300 U/L, 301 U/L, 302 U/L, 303 U/L, 304 U/L, 305 U/L, 306 U/L, 307 U/L, 308 U/L, 309 U/L, 310 U/L, 311 U/L, 312 U/L, 313 U/L, 314 U/L, 315 U/L, 316 U/L, 317 U/L, 318 U/L, 319 U/L, 320 U/L, 321 U/L, 322 U/L, 323 U/L, 324 U/L, 325 U/L, 330 U/L, 340 U/L, 350 U/L, 400 U/L, and 500 U/L).

IIaiii. Aspartate Aminotransferase

In some embodiments, a patient is determined to exhibit cholestasis or one or more symptoms thereof and is administered an anti-cholestatic agent when the patient exhibits an AST level, as measured in a LFT, that is greater than the norm.

In some embodiments, a is determined to exhibit cholestasis or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIaiv. Alanine Aminotransferase

In some embodiments, a patient is determined to exhibit cholestasis or one or more symptoms thereof and is administered an anti-cholestatic agent when the patient exhibits an ALT level, as measured in a LFT, that is greater than the norm.

In some embodiments, a patient is determined to exhibit cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

Recommended Clinical Parameters for Determining that a Patient Exhibits Hyperbilirubinemia or a Symptom Thereof

### Bilirubin Test

In some embodiments, a patient is determined to exhibit hyperbilirubinemia or one or more symptoms thereof and is administered an anti-cholestatic agent when the patient exhibits a bilirubin level, as measured in a blood test (e.g., a bilirubin test), that is greater than the norm.

In some embodiments, a patient is determined to exhibit hyperbilirubinemia or one or more symptoms thereof and is administered an anti-cholestatic agent when the patient's total bilirubin level is greater than 1.2 mg/dL (e.g., 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3 mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, a patient is determined to exhibit hyperbilirubinemia or one or more symptoms thereof and is administered an anti-cholestatic agent when the patient's direct bilirubin level is greater than 0.2 mg/dL (e.g., 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3 mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, the patient is determined to exhibit hyperbilirubinemia or one or more symptoms thereof and is administered an anti-cholestatic agent when the patient exhibits a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3 mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Evaluating Safety and Efficacy of a Pseudotyped AAV8 Vector Including a Nucleic Acid Sequence Encoding a Myotubularin 1 Gene in a Murine Model of X-Linked Myotubular Myopathy

Objective and Study Methodology The objective of this study was to perform an efficacy and sampling study using myotubularin 1 (MTM1) hemizygous (“HEMI”) knock out (KO) mice and determine the possible effects of liver-specific MTM1 expression in mice. The mice were dosed with different test articles intravenously at ~4 weeks of age. Mice were sampled at ~16 weeks of age (~12 weeks post-dosing). Tissue samples were used for myotubularin and bile salt transporter expression, bile acid level measurements, and histopathology. Whole blood samples were used for hematology analysis and serum samples were used for clinical chemistry analysis. In addition, a group of naïve MTM1 KO mice were sampled at ~5 weeks of age, and tissues were used for histopathology.

This study was performed according to appropriate methodologies and standard operating procedures (SOPs) at the testing facility or test sites. This study was compliant with standard ethics of animal welfare.

### Materials and Methods

This study used MTM1 KO mice, with 28 male wild type (WT) mice and 70 male KO mice. Animals were sampled at ~5 and ~16 weeks. Each animal was permanently identified according to SOPs of the Testing Facility with unique permanent identification numbers. Animals were identified using toe and tail numbering and ear marks. There was a ~3-4-day acclimation period before dosing. Animals were housed in groups of up to 3-4 per cage, and at least one WT mouse was placed in each cage for socializing purposes. Individually ventilated caging systems (IVC) and polycarbonate Type II Long cages were used to house the mice.

Veterinary care was available throughout the course of the study and animals were examined by the responsible trained personnel and supervised by veterinarian as warranted by clinical signs or other changes.

Before the initiation of the in-life phase, any animals considered unsuitable for use in the study were replaced by alternate animals obtained from the same shipment and maintained under the same environmental conditions. Animals in poor health or at extremes of body weight range were not assigned to groups. In setting up groups for this study, KO mice were randomized into groups so that whole litters of mice did not end up in a single testing group to avoid a “litter effect”. Mice were housed in groups of up to 4-5. Each cage included 3-4 KO mice, and 1-2 WT mice for socializing purposes. None of the KO mice were housed individually.

Four articles were tested: 1) an AAV8 vector containing a mouse MTM1 (mMTM1) gene under the control of a desmin promoter (AAV8-Des-mMTM1), 2) an AAV8 vector containing mMTM1 under the control of an APoE-A1 AT promoter (AAV8-APoE-A1 AT-mMTM1), 3) an AAV8 vector containing a human MTM1 (hMTM1) gene with a stop codon under the control of a desmin promoter (AAV8-Des-hMTM1-STOP), and 4) an AAV8 empty capsid. A vehicle control was used containing a placebo of Ringer's Lactate solution with 0.01% pluronic. Dose levels were selected based on previous studies with mice. The high dose was a multiple of the previous high dose in mice, as the previous high dose showed significant pharmacological effect without toxicity. Animals were dosed by slow intravenous (IV) bolus injection into a tail vein. Dose volumes (total 16.33 mL/kg) were administered as a split IV bolus dose with 2 to 3 hours between administrations. The dose site, end time, and dose volume were documented. Animals were anesthetized with isoflurane, if necessary.

This administration route is consistent with the proposed route of administration in humans and is expected to provide appropriate systemic exposure for investigation and associated pharmacological activity. The administration frequency was one dose on day 1. Dose levels were anticipated to identify achievable associated pharmacological activity. Necropsy occurred ~12 weeks post dosing.

Treatment/Study groups are summarized in Table 4, below. Additionally, a group of 10 naïve MTM1 KO mice were sampled at ~5 (5.3) weeks of age.

TABLE 4
Mouse study groups

| | | Animal | Treatment | Dose | Volume | Sampling | Dosing |
|---|---|---|---|---|---|---|---|
| Group | Genotype | number | Group | (vg/kg) | (ml/kg) | Age (wks) | Age (wks) |
| 1 | Wild Type | 14 | Vehicle | 0 | 16.33 | ~16.4 | ~4.1 |
| 2 | MTM1 KO | 14 | AAV8-Des- | 4e14 vg/kg | 16.33 | ~16.3 | ~4.1 |
| | (HEMI) | | mMTM1 | | | | |
| 3 | MTM1 KO | 13 | AAV8-Des- | 4e14 vg/kg + | 16.33 | ~16.4 | ~4.2 |
| | (HEMI) | | mMTM1 + | 2e14 vg/kg | | | |
| | | | AAV8-Des- | | | | |
| | | | hMTM1- | | | | |
| | | | STOP | | | | |
| 4 | MTM1 KO | 14 | AAV8-Des- | 4e14 vg/kg + | 16.33 | ~16.6 | ~4.1 |
| | (HEMI) | | mMTM1 + | equiv cap/kg | | | |
| | | | AAV8 | as G3 | | | |
| | | | empty | | | | |
| | | | capsid | | | | |
| 5 | MTM1 KO | 14 | AAV8-Des- | 4e14 vg/kg + | 16.33 | ~16.7 | ~4.2 |
| | (HEMI) | | mMTM1 + | 2e14 vg/kg | | | |
| | | | AAV8- | | | | |
| | | | APoE- | | | | |
| | | | A1AT- | | | | |
| | | | mMTM1 | | | | |
| 6 | MTM1 KO | 12 | AAV8-Des- | 4e14 vg/kg + | 16.33 | ~16.8 | ~4.1 |
| | (HEMI) | | mMTM1 + | 6e13 vg/kg + | | | |
| | | | AAV8- | 1.4e14 vg/kg | | | |
| | | | APoE- | | | | |
| | | | A1AT- | | | | |
| | | | mMTM1 + | | | | |
| | | | AAV8- | | | | |
| | | | Des- | | | | |
| | | | hMTM1- | | | | |
| | | | STOP | | | | |
| 7 | Wild Type | 14 | AAV8-Des- | 4e14 vg/kg + | 16.33 | ~16.4 | ~4.1 |
| | | | mMTM1 + | 2e14 vg/kg | | | |
| | | | AAV8-Des- | | | | |
| | | | hMTM1- | | | | |
| | | | STOP | | | | |

Test article volumes used prior to dosing are summarized in Table 5, below.

TABLE 5
Test article volumes prior to dosing

| | | | | AAV8- | | | | | |
|---|---|---|---|---|---|---|---|---|---|
| | | | | Des- | | AAV8- | | | |
| | | | AAV8- | hMTM | AAV8 | APoE- | | | |
| | | | Des- | 1- | empty | A1AT- | Placebo | Total | Total |
| | Treatment | Dose | mMTM1 | STOP | capsid | mMTM1 | volume | Volume | Volume |
| Group | Group | (vg/kg) | (mL) | (mL) | (mL) | (mL) | (ml) | (mL) | (ml/kg) |
| 1 | Vehicle | 0 | NA | NA | NA | NA | 5 | 5 | 16.33 |
| 2 | AAV8-Des- | 4e14 | 2.25 | NA | NA | NA | 2.75 | 5 | 16.33 |
| | mMTM1 | vg/kg | | | | | | | |
| 3 | AAV8-Des- | 4e14 | 2.25 | 1.05 | NA | NA | 1.7 | 5 | 16.33 |
| | mMTM1 + | vg/kg + | | | | | | | |
| | AAV8-Des- | 2e14 | | | | | | | |
| | hMTM1-STOP | vg/kg | | | | | | | |
| 4 | AAV8-Des- | 4e14 | 2.25 | NA | 2.75 | NA | NA | 5 | 16.33 |
| | mMTM1 + | vg/kg + | | | | | | | |
| | AAV8 empty | equivalent | | | | | | | |
| | capsid | cp/kg | | | | | | | |
| | | as | | | | | | | |
| | | Group 3 | | | | | | | |
| 5 | AAV8-Des- | 4e14 | 2.25 | NA | NA | 0.5 | 2.25 | 5 | 16.33 |
| | mMTM1 + | vg/kg + | | | | | | | |
| | AAV8-APoE- | 2e14 | | | | | | | |
| | A1AT-mMTM1 | vg/kg | | | | | | | |
| 6 | AAV8-Des- | 4e14 | 2.25 | 0.74 | NA | 0.15 | 1.86 | 5 | 16.33 |
| | mMTM1 + | vg/kg + | | | | | | | |
| | AAV8-APoE- | 6e13 | | | | | | | |
| | A1AT-mMTM1 + | vg/kg + | | | | | | | |
| | AAV8-Des- | 1.4e14 | | | | | | | |
| | hMTM1-STOP | vg/kg | | | | | | | |
| 7 | AAV8-Des- | 4e14 | 2.25 | 1.05 | NA | NA | 1.7 | 5 | 16.33 |
| | mMTM1 + | vg/kg + | | | | | | | |
| | AAV8-Des- | 2e14 | | | | | | | |
| | hMTM1-STOP | vg/kg | | | | | | | |

The following humane endpoint criteria are applicable for the study. If the mice met the predefined humane endpoint criteria, they underwent a planned euthanasia for welfare reasons. If any of the mice needed to be euthanized for welfare reasons, they were opened, and macroscopic observations were recorded. In case the general health status of an animal deteriorated significantly, a shortened tissue isolation protocol was applied, if possible. The shortened tissue isolation protocol consisted of collecting a skeletal muscle (quadriceps) and 1 liver lobe in liquid nitrogen and 1 liver lobe and heart (dorsal portion) tissue in formalin. The skeletal muscle and liver lobe were stored at −80° C. while the liver lobe and heart (dorsal portion) in formalin were stored at room temperature. Due to a limited staff capacity during evenings and weekends to perform sampling on acute cases needing immediate action, mice were euthanized by an overdose of CO₂ and decapitated. In this case, no sampling was performed.

Body weight was measured three times a week and mortality were recorded. If surviving to the end of the study, at endpoint, at ~16-17 weeks of age (~12-13 weeks after dosing), the mice were euthanized by deep anesthetization with sodium pentobarbital (180 mg/kg). As much whole blood was collected as possible via cardiac puncture. Blood was first collected for hematology (100 μL of EDTA whole blood), and then for clinical chemistry (200 μL of whole blood to separate minimum 80 μL of serum). Any remaining serum was collected. Then, the mice were transcardially perfused with heparinized saline to remove blood from the tissues. The group of 10 naïve MTM1 KO mice were sampled at ~5 wks of age. Sampling included blood sampling (whole blood and serum), perfusion, and dissection of samples for histology and histopathology. No samples were taken for other analyses.

Tissue samples were used for myotubularin and bile salt transporter expression, bile acid level measurements, and histopathology. In-life and endpoint whole blood samples were used for hematology analysis and serum samples for clinical chemistry analysis. The same portion of the sample from each mouse was used for each analysis. The heart was collected with the left and right atria, then halved in the frontal plane. Tissue collection and analysis is summarized in Table 6, below.

TABLE 6
Mouse tissue collection and analysis

| | | Myotubularin and | Myotubularin (ELISA), | |
|---|---|---|---|---|
| | | Bile Salt | Vector Copy Number | |
| | Histology and | Transporter | (VCN) and/or Bile acid | Extra |
| Tissue | Histopathology | analysis by IHC | Analysis (ELISA) | samples |
| Quadriceps | L. muscle (½) | L. muscle (½) | R. muscle | NA |
| Diaphragm | 1 sample | 1 sample | 1 sample | NA |
| Liver | left lateral lobe | left medial lobe and | Remaining lobes (right | NA |
| | and a small part | remaining right | lobe, right lateral lobe, | |
| | of the right | medial lobe | caudate lobe) | |
| | medial lobe | | | |
| | (with gall | | | |
| | bladder) | | | |
| Heart | ½ heart (dorsal) | NA | ½ heart | NA |
| | | | (ventral) split in 2 samples | |
| | | | (sample 1, vial 1; sample | |
| | | | 2, vial 2 for each animal; | |
| | | | same portion) | |
| Triceps (R) | NA | NA | NA | Whole muscle |
| Gastrocnemius (R) | | | | |
| Tibialis | | | | |
| anterior (R) | | | | |
| Lungs | 1 sample | NA | NA | NA |
| Kidneys | L + R, 2 samples | NA | NA | NA |
| Spleen | 1 sample | NA | NA | NA |
| Brain | 1 sample | NA | NA | NA |

### Histology and Histopathology

The following tissue samples were dissected, and placed in 10% neutral buffered formalin:
Quadriceps (L. muscle, ½)
Diaphragm (1 sample)
Liver (left lateral lobe and a small part of the right medial lobe (with gall bladder))
Heart (½, dorsal)
Lungs (1 sample)
Kidneys (L+R, 2 samples)
Spleen (1 sample)
Brain (1 sample)

Samples were embedded in paraffin, sectioned to slide, and stained with hematoxylin and eosin (H&E) for microscopic evaluation.
Sample Collection for: Myotubularin (ELISA), Vector Copy Number (VCN), and/or Bile Acid Analysis (ELISA); Myotubularin and Bile Acid Salt Transporter Analysis by Immunohistochemistry (IHC); and Reserve Tissues

Sterile Collection for bioanalytical analysis (Myotubularin (ELISA), Vector Copy Number (VCN) and/or Bile Acid Analysis (ELISA); Myotubularin and Bile Acid Salt Transporter Analysis by IHC; and Reserve Tissues): Each tissue was collected in RNAse-free screw top polypropylene tubes, flash frozen in liquid nitrogen, under sterile conditions, and stored at ~80° C. according to Testing Facility SOPs. Samples were stored on dry ice prior to storage. Scalpel blades, weigh boats (small dish), and dissecting instruments were replaced/cleaned prior to handling a new mouse. All instruments were wiped with RNAse Zap (or similar) between collection of each tissue. Precautionary measures were in place to prevent cross-contamination during tissue collection.

Myotubularin (ELISA), Vector Copy Number (VCN) and/or Bile Acid Analysis (ELISA)

The following tissue samples were dissected and frozen in liquid nitrogen:
Quadriceps (R. muscle)
Diaphragm (1 sample)
Liver (right lobe, right lateral lobe, caudate lobe)
Heart (½, ventral) (split in two samples)

### Myotubularin and Bile Acid Salt Transporter Analysis by IHC

The following tissue samples were dissected and frozen in liquid nitrogen:
Quadriceps (L. muscle, ½)
Diaphragm (1 sample)
Liver (left medial lobe and remaining right medial lobe without gall bladder)

### Reserve Tissues

The following tissue samples were dissected and frozen in liquid nitrogen:
Triceps (R. muscle)
Gastrocnemius (R. muscle)
Tibialis anterior (R. muscle)

### Clinical Chemistry Panel

At in-life sampling and terminal necropsy, a clinical biochemistry panel of markers was analyzed in unhemolysed serum for the following parameters: alanine aminotransferase (ALAT), aspartate aminotransferase (ASAT), alkaline phosphatase (AFOS), gamma-glutamyl transferase (GGT), lactate dehydrogenase (LDH), creatine kinase (CK), albumin, total bilirubin, total protein, creatinine, bile acids, calculated globulin, calculated albumin/globulin ratio, and urea nitrogen. If the blood sample was limited, analysis priority was given in the following order:
1. Alanine aminotransferase (ALAT)
2. Total/Direct bilirubin
3. Bile acids
4. Aspartate aminotransferase (ASAT)
5. Alkaline phosphatase (AFOS)
6. Gamma-glutamyl transferase (GGT)
7. Lactate dehydrogenase (LDH)
8. Creatine kinase (CK)
9. Albumin
10. Total protein
11. Creatinine
12. Urea Nitrogen
Only ALAT was analyzed from in-life samples due to limited serum sample.

### Hematology Panel

At in-life sampling and terminal necropsy, a hematology panel of markers was analyzed in EDTA whole blood for the following parameters: hemoglobin, red blood cell counts, white blood cell counts including relative and absolute differential counts, absolute reticulocyte counts, reticulocyte %, and thrombocyte counts. Additional parameters, if assay quantity of whole blood allowed, included: hematocrit, mean corpuscular volume, mean corpuscular hemoglobin, and mean corpuscular hemoglobin concentration.

### Genotyping Samples

A tail sample was taken at the endpoint sampling for possible regenotyping with regular PCR.

### Statistical Analysis and Data Graphical Presentation

Prior to further statistical analysis, data quality checks and validations were performed. During that process, potential outliers were identified and assessed. No outliers were removed from the data without a clear justification for removal (e.g., identified measurement error in the laboratory notes).

The planned comparisons in this study were:
Group 1 vs. Group 2 and Group 7
Group 2 vs. Groups 3-6

The assumption of normality of each data set was primarily based on experience (e.g., data within a population is known to be approximately Gaussian) and observations during the validation phase. Some biological variables are known to follow lognormal distributions—in these cases, the data is first transformed to logarithms, and then a parametric statistical test can be used. The same normality assumption was used for a series of experiments or a group of similar readouts from an assay. In addition, the D'Agostino-Pearson omnibus normality test was used for clinical chemistry and hematology data to support the decision whether parametric or nonparametric tests were to be used. The summary of readouts for statistical analysis and visualization is summarized in Table 7, below.

TABLE 7
Summary of readouts for statistical analysis and visualization

| Test/Assay | Normalization | Visualization |
|---|---|---|
| Body weight | No | Line graph |
| Hematology | No | Bar graph |
| Clinical chemistry | No | Bar graph |

Descriptive statistics, including group size, mean, SD, and SEM were provided for each parameter.

### Statistical Analysis of Single Time Point Data

Simple comparisons between two groups were performed using unpaired Welch's t-test, or, when the assumption of normality or lognormality was not met, by the Mann-Whitney U-test.

### Repeated Observations of the Same Subjects (Longitudinal Data)

Comparisons between the two groups were performed using a two-way Mixed-effects model (Mixed Anova) with Geisser-Greenhouse correction (not assuming sphericity), followed by Fisher's LSD test between the two groups performed for each time point. Time, group, and the interaction of Time×Group were the fixed effects, and the individual subject was the random effect.

In the case of longitudinal comparison of three or more groups, the two-way Mixed-effects model with Geisser-Greenhouse correction, followed by Dunnett's multiple comparisons test was performed. One family per time point was set. Time, group, and the interaction of Time×Group were the fixed effects, and the individual subject was the random effect.

The following sections provide detailed results for certain of the parameters mentioned in Table 6 above.

### Results

Results of this study provided an indication of safety and efficacy of the compositions and methods described above.

### Body Weight

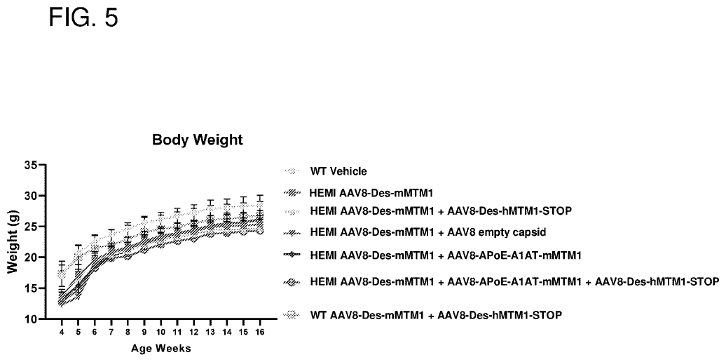

Body weight was significantly lower in HEMI AAV8-Des-mMTM1 mice compared to WT Vehicle mice (Two-Way-ANOVA: Group: p<0.0001) at all time points (Fisher's LSD multiple comparison: p<0.001, for all) (FIG. 5).

Body weight was significantly less in WT AAV8-Des-mMTM1+AAV8-Des-hMTM1-STOP mice compared to WT Vehicle mice (Two-Way-ANOVA: Group: p<0.05) on weeks 7-16 (Fisher's LSD multiple comparison: p<0.05, for all) (FIG. 5).

Body weight was significantly altered among HEMI mice (Two-Way-ANOVA: Group effect: NS, Group×Time interaction effect: p<0.0001). Post-hoc comparisons revealed that body weight of HEMI AAV8-Des-mMTM1+AAV8 empty capsid mice were significantly decreased on weeks 4 and 5 (Dunnett's multiple comparison: p<0.05, for both) and body weight of HEMI AAV8-Des-mMTM1+AAV8-APoE-A1AT-mMTM1+AAV8-Des-hMTM1-STOP mice were significantly decreased on weeks 5-6 and 8-9 compared to HEMI AAV8-Des-mMTM1 mice (Dunnett's multiple comparison: p<0.05, for all).

### Mortality

No data from animals that died or were euthanized prior to dosing is shown. No mortality was observed in groups 1-3, 5, and 7. Two mice in each of groups 4, 6, and naïve were found dead prior to dosing due to factors unrelated to the test articles.

### Clinical Chemistry

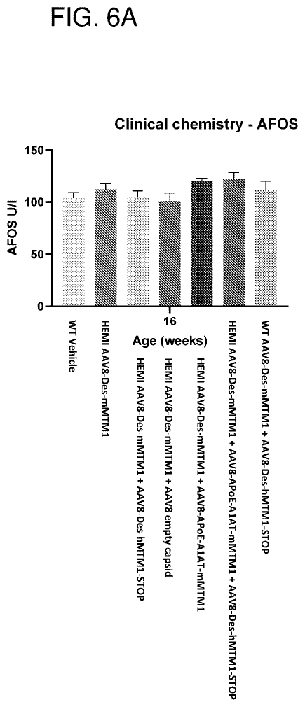

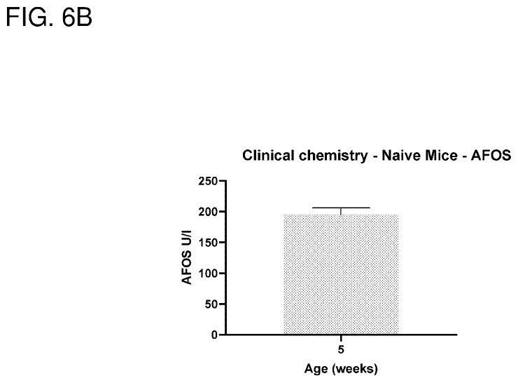

All the clinical chemistry assessments were performed only from terminal samples (week 16 or week 5 for naïve mice) for all groups, with the exception of ALAT that was analyzed from both in-life and terminal samples (weeks 4-10). Data from clinical chemistry analysis is shown in FIGS. 6A-19B.

### Alkaline Phosphatase (AFOS)

There were no statistically significant differences between the groups in AFOS levels at 16 weeks of age (FIG. 6A).

AFOS levels from naïve mice at 5 weeks of age are shown in FIG. 6B.

### Alanine Aminotransferase (ALA T)

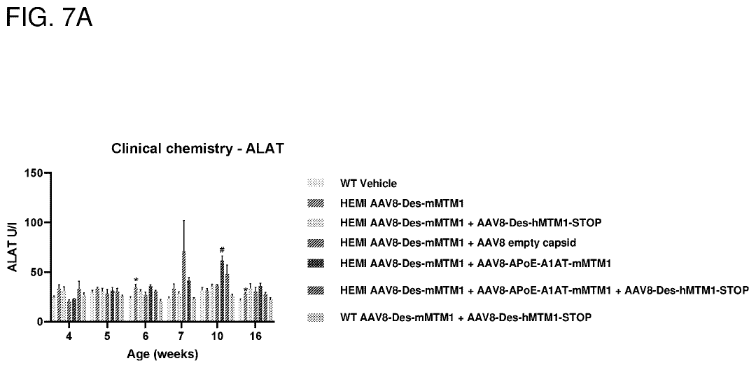

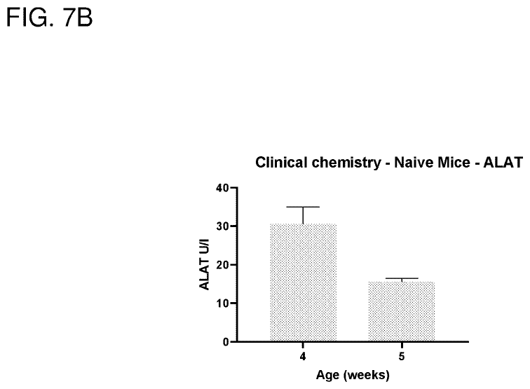

ALAT levels were significantly increased in HEMI AAV8-Des-mMTM1 mice compared to WT Vehicle mice at 6-and 16-week time points (Two-Way-ANOVA: Group effect: p=0.0005) (Fisher's LSD multiple comparison: p<0.05, for both) (FIG. 7A).

ALAT levels were significantly increased at 10 weeks of age in HEMI AAV8-Des-mMTM1+AAV8-APoE-A1AT-mMTM1 mice compared to HEMI AAV8-Des-mMTM1 mice (One-Way ANOVA: Group effect: p=0.0026, Dunnett's post-hoc comparison: p<0.05) (FIG. 7A).

ALAT levels from naïve mice at 5 weeks of age are shown in FIG. 7B.

### Aspartate Aminotransferase (ASA T)

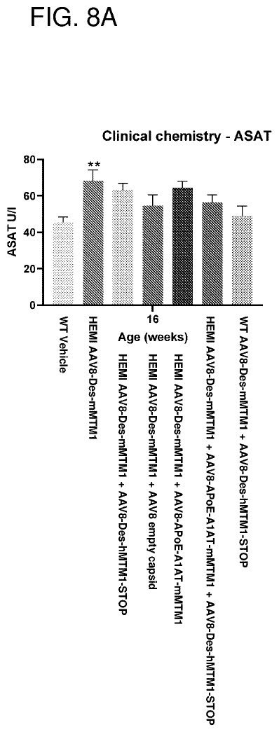

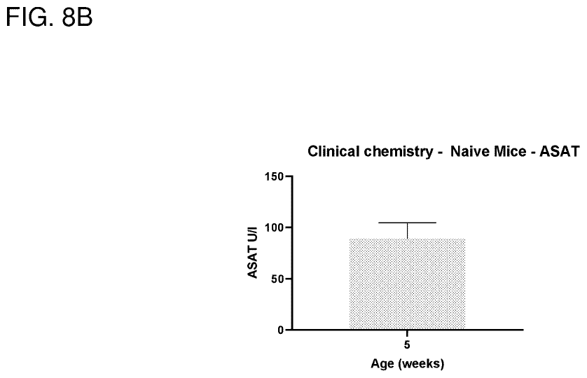

ASAT levels were significantly increased in HEMI AAV8-Des-mMTM1 mice compared to WT Vehicle mice at 16 weeks of age (t-test: p<0.01) (FIG. 8A).

ASAT levels from naïve mice at 5 weeks of age are shown in FIG. 8B.

### Albumin

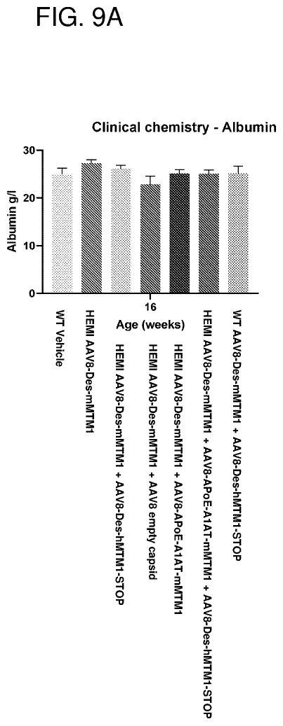

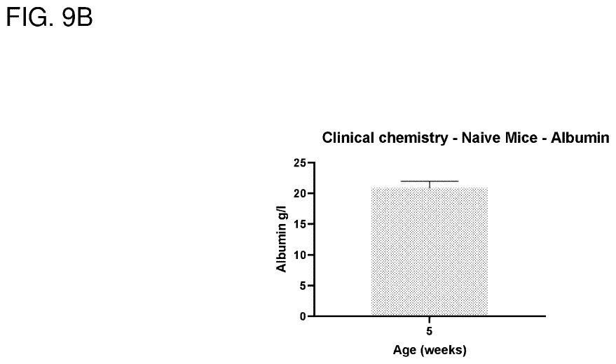

There were no statistically significant differences between the groups in albumin levels at 16 weeks of age (FIG. 9A).

Albumin levels from naïve mice at 5 weeks of age are shown in FIG. 9B.

### Gamma-Glutamyl Transferase (GGT)

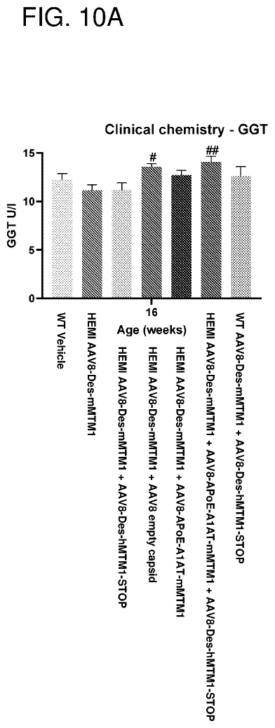

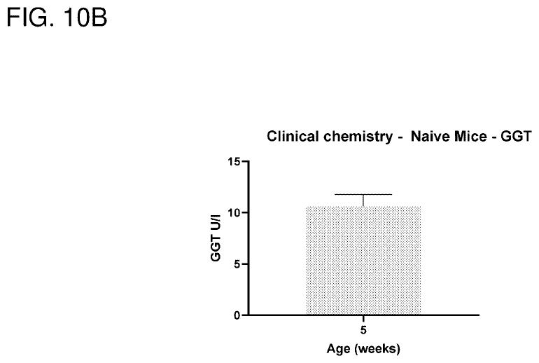

GGT levels were significantly increased in HEMI AAV8-Des-mMTM1+AAV8 empty capsid mice and HEMI AAV8-Des-mMTM1+AAV8-APoE-A1 AT-mMTM1+AAV8-Des-hMTM1-STOP mice compared to HEMI AAV8-Des-mMTM1 mice at 16 weeks of age (One-Way ANOVA: Group effect: p=0.0013, Dunnett's post-hoc comparison: p<0.05, for both) (FIG. 10A).

GGT levels from naïve mice at 5 weeks of age are shown in FIG. 10B.

### Total Protein

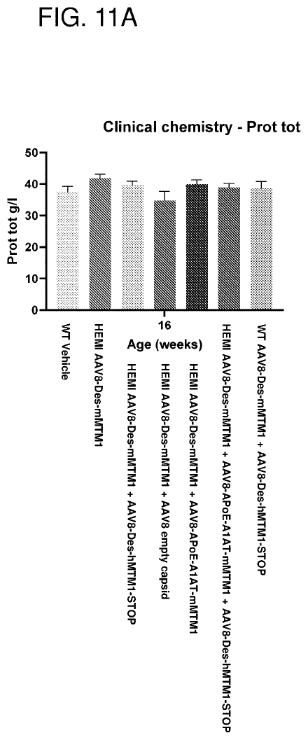

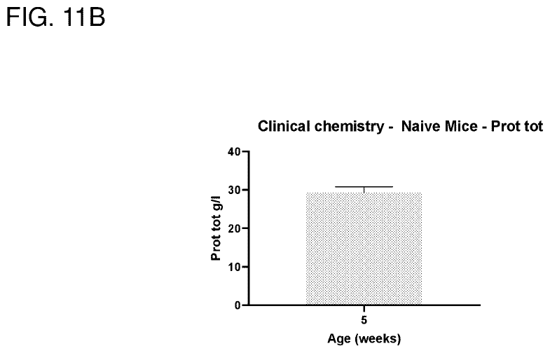

There were no statistically significant differences between the groups in total protein levels at 16 weeks of age (FIG. 11A).

Total protein levels from naïve mice at 5 weeks of age are shown in FIG. 11B.

### Urea

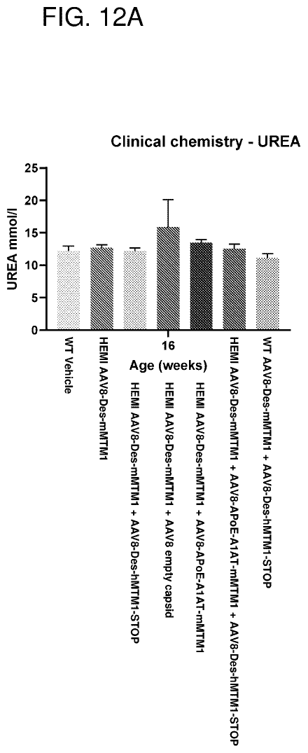

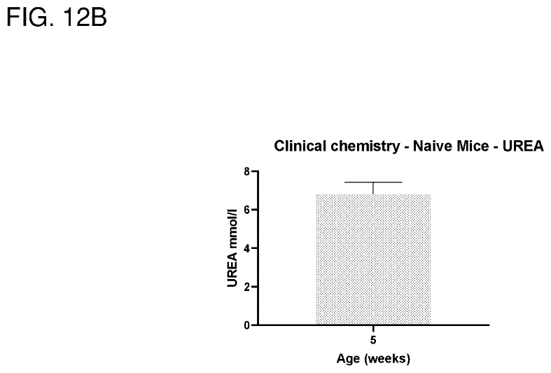

There were no statistically significant differences between the groups in urea levels at 16 weeks of age (FIG. 12A).

Urea levels from naïve mice at 5 weeks of age are shown in FIG. 12B.

### Lactate Dehydrogenase (LDH)

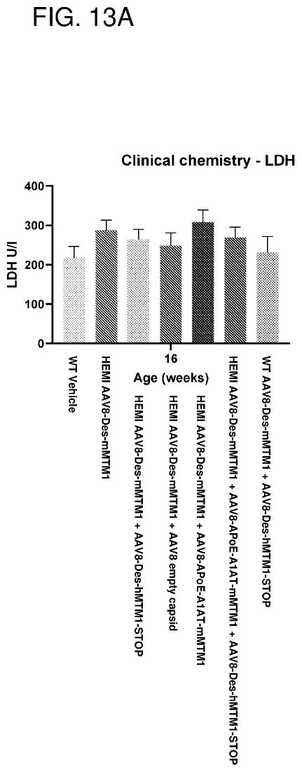

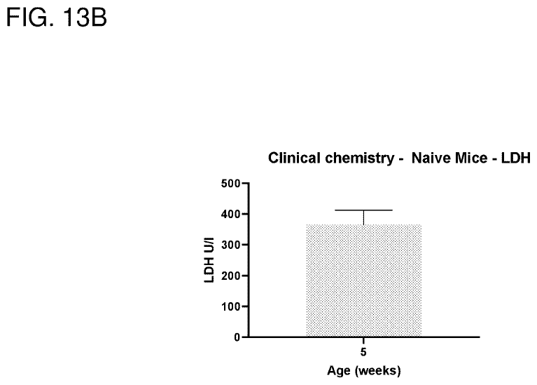

There were no statistically significant differences between the groups in LDH levels at 16 weeks of age (FIG. 13A).

LDH levels from naïve mice at 5 weeks of age are shown in FIG. 13B.

### Bile Acids

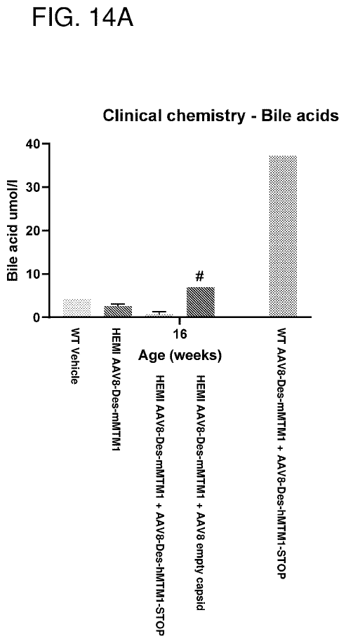

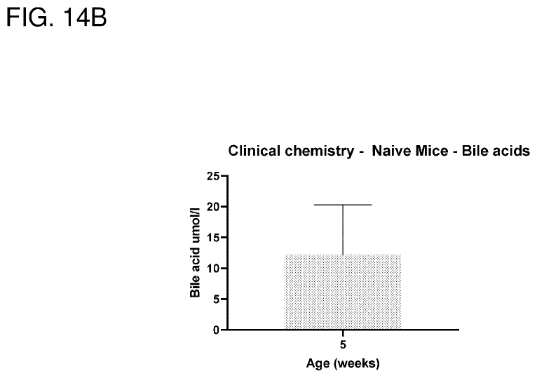

Bile acids levels were significantly increased in HEMI AAV8-Des-mMTM1+AAV8 empty capsid mice compared to HEMI AAV8-Des-mMTM1 mice at 16 weeks of age (One-Way ANOVA: Group effect: p=0.0258, Dunnett's post-hoc comparison: p<0.05) (FIG. 14A). However, it should be noted that most of the bile acids values were under the detection level in all treatment groups.

Bile acid levels from naïve mice at 5 weeks of age are shown in FIG. 14B.

### Total Bilirubin

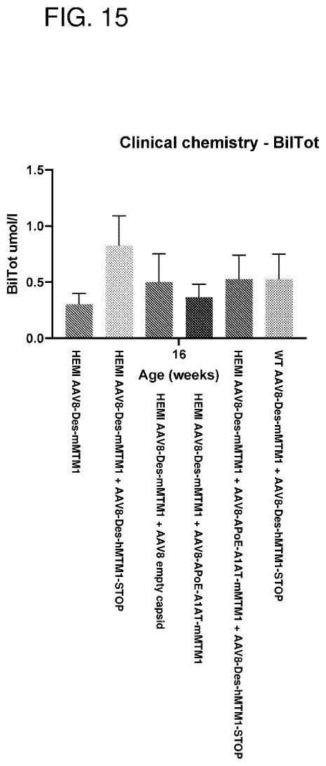

There were no statistically significant differences between the groups in total bilirubin levels at 16 weeks of age (FIG. 15).

No total bilirubin data was available from naïve mice at 5 weeks of age (all values were below the detection level).

### Calculated Globulin

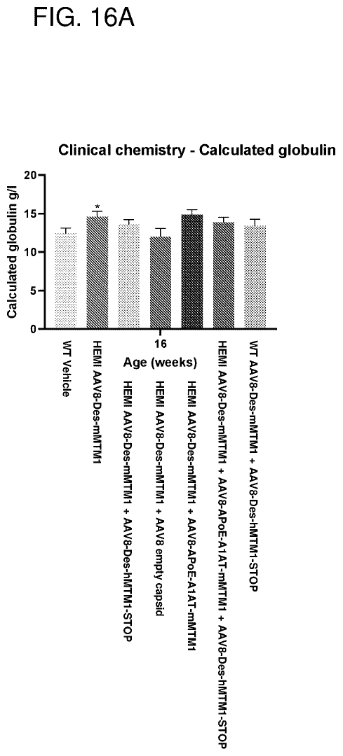

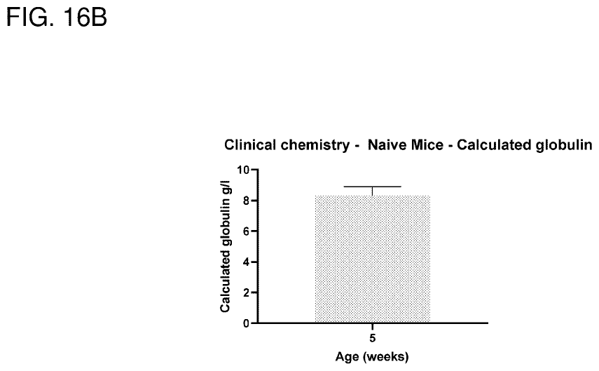

Calculated globulin levels were significantly increased in HEMI AAV8-Des-mMTM1 mice compared to WT Vehicle mice at 16 weeks of age (t-test: p<0.05) (FIG. 16A).

Calculated globulin levels from naïve mice at 5 weeks of age are shown in FIG. 16B.

### Calculated Albumin/Globulin Ratio

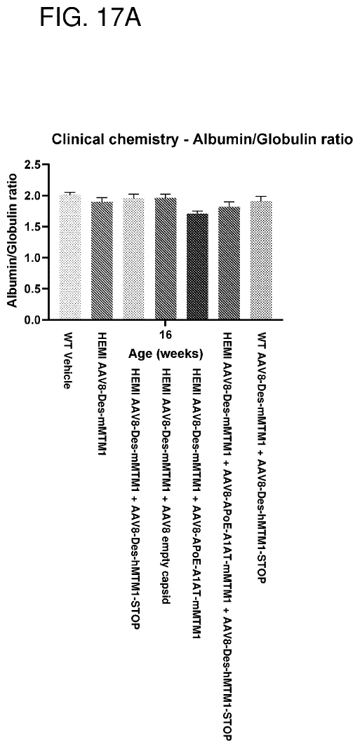

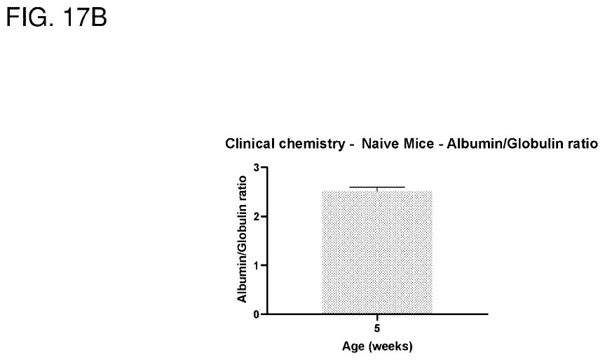

There were no statistically significant differences between the groups in calculated albumin/globulin ratio levels at 16 weeks of age (FIG. 17A).

Albumin/Globulin ratio levels from naïve mice at 5 weeks of age are shown in FIG. 17B.

### Creatine Kinase

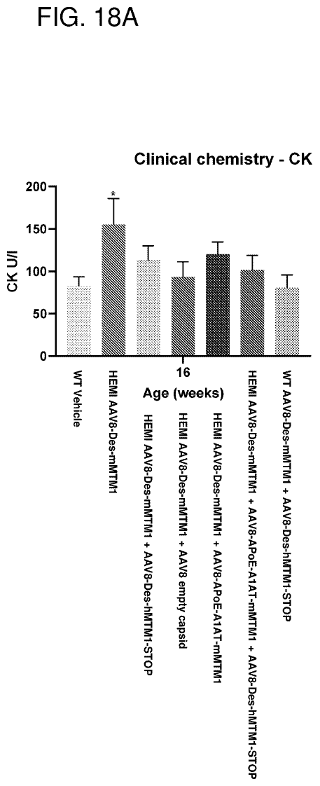

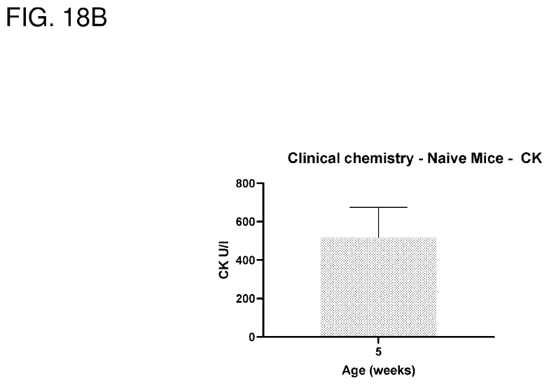

CK levels were significantly increased in HEMI AAV8-Des-mMTM1 mice compared to WT Vehicle mice at 16 weeks of age (t-test: p<0.05) (FIG. 18A).

CK levels from naïve mice at 5 weeks of age are shown in FIG. 18B.

### Creatinine

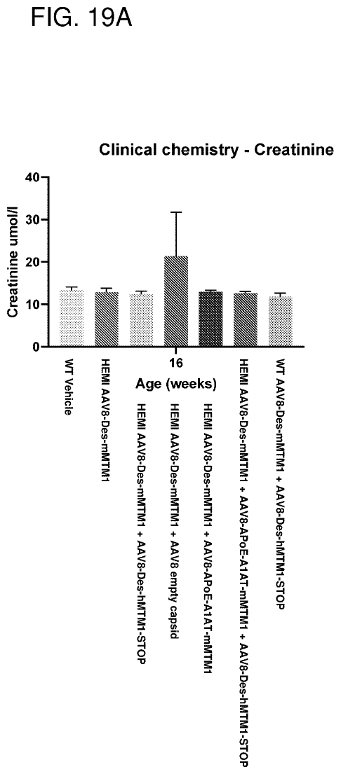

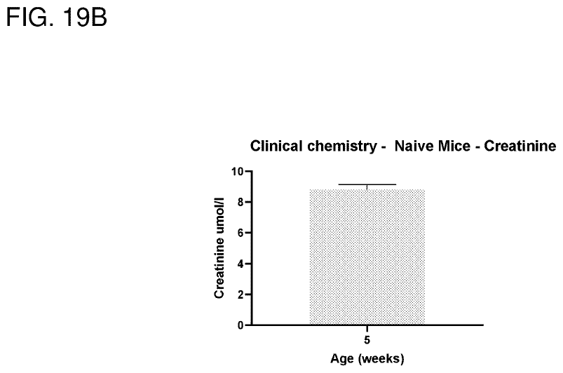

There were no statistically significant differences between the groups in creatinine levels at 16 weeks of age (FIG. 19A).

Creatinine levels from naïve mice at 5 weeks of age are shown in FIG. 19B.

### Hematology

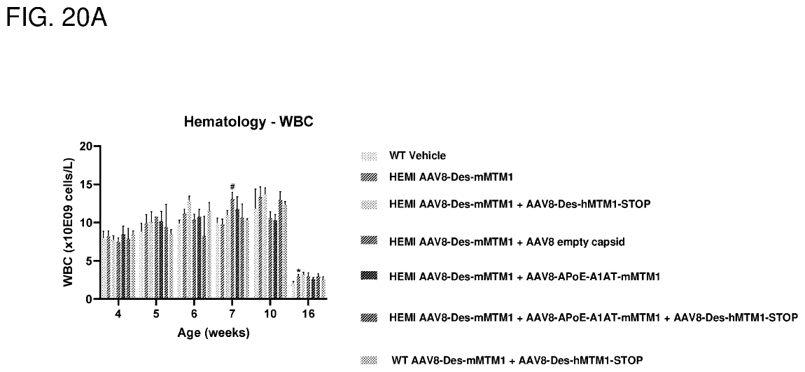

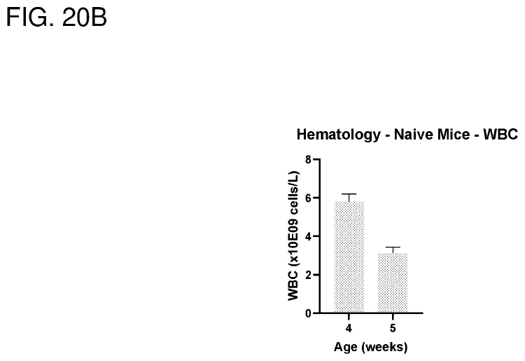

Due to different blood sampling techniques between in-life sampling (saphenous vein, no anesthesia) and terminal sampling (cardiac puncture, pentobarbital anesthesia), statistical analysis was performed separately for in-life samples (weeks 4-10) and terminal samples (week 16). It has been reported that significant differences in blood parameters, for example, the number of total white blood cells, result from different blood sampling techniques (Hoggatt et al. Exp Hematol. 44(2):132-137.e1. (2016)). Data from hematology analysis is shown in FIGS. 20A-41B.

### White Blood Cell Count (WBC)

White blood cell counts were significantly increased in HEMI AAV8-Des-mMTM1 mice compared to WT Vehicle mice at 16 weeks of age (t-test, p<0.05) (FIG. 20A). White blood cell counts were significantly increased in HEMI AAV8-Des-mMTM1+AAV8 empty capsid mice at 7 weeks of age compared to HEMI AAV8-Des-mMTM1 mice (Two-Way ANOVA: Group effect: NS, Group×Time interaction effect: p<0.05, Dunnett's post-hoc comparison: p<0.05) (FIG. 20A).

White blood cell counts from naïve mice at 4 and 5 weeks of age are shown in FIG. 20B.

### Red Blood Cell Count (RBC)

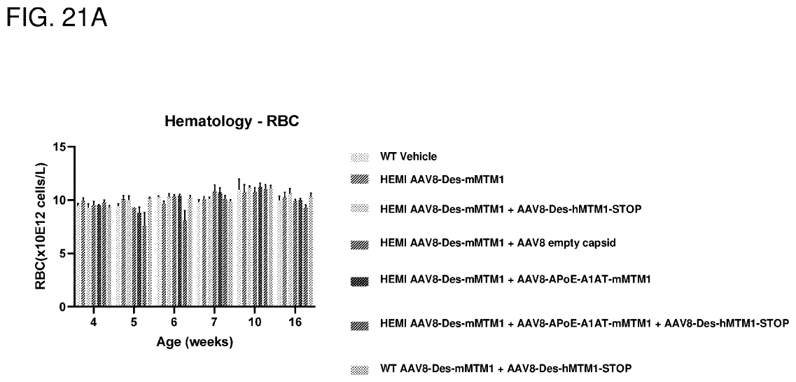

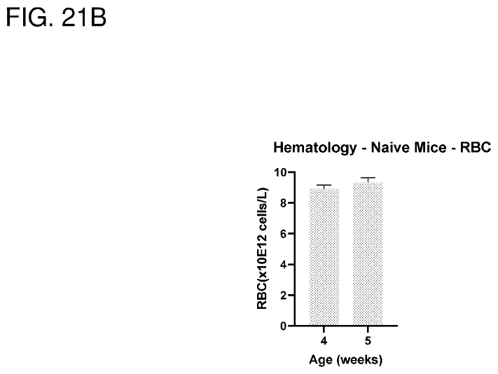

There were no statistically significant differences between the groups in red blood cell counts (FIG. 21A).

Red blood cell counts from naïve mice at 4 and 5 weeks of age are shown in FIG. 21B.

### Hemoglobin (HGB)

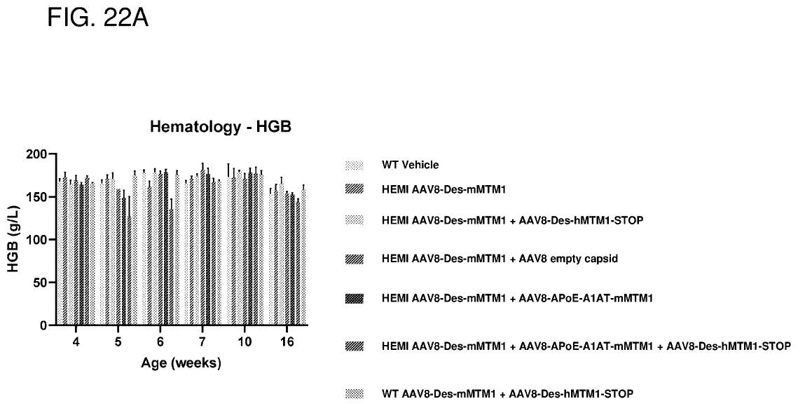

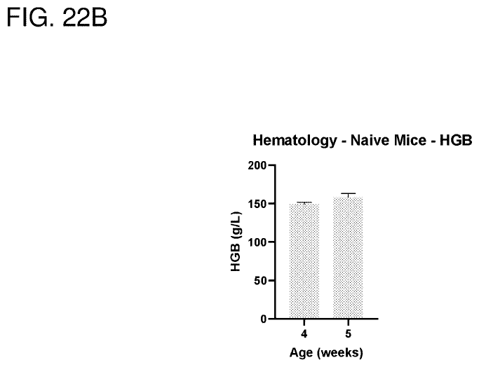

There were no statistically significant differences between the groups in hemoglobin levels (FIG. 22A).

Hemoglobin levels from naïve mice at 4 and 5 weeks of age are shown in FIG. 22B.

### Hematocrit (HCT)

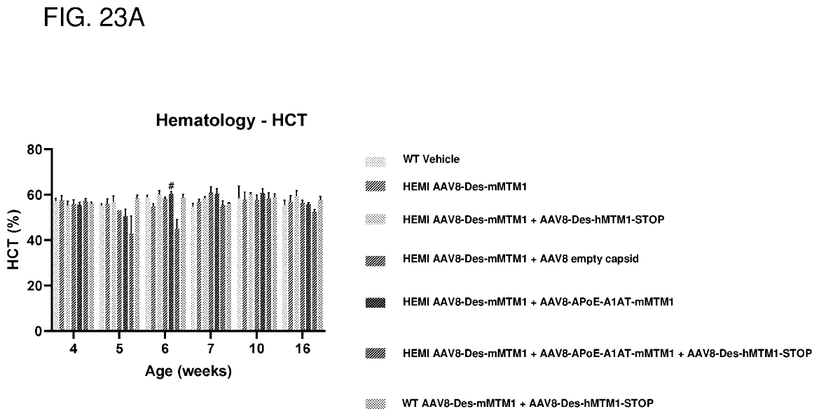

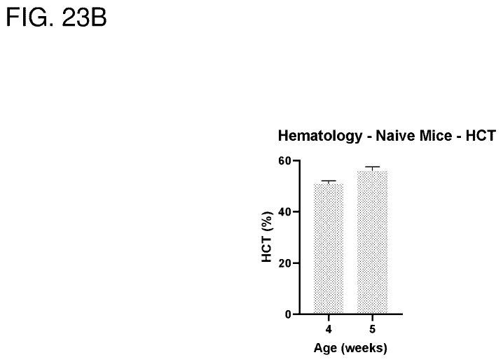

Hematocrit levels were significantly increased in HEMI AAV8-Des-mMTM1+AAV8-APoE-A1AT-mMTM1 mice compared to HEMI AAV8-Des-mMTM1 mice at 6 weeks of age (Two-Way ANOVA: Group effect: p<0.05, Dunnett's post-hoc comparison: p<0.05) (FIG. 23A).

Hematocrit levels from naïve mice at 4 and 5 weeks of age are shown in FIG. 23B.

### Mean Corpuscular Volume (MCV)

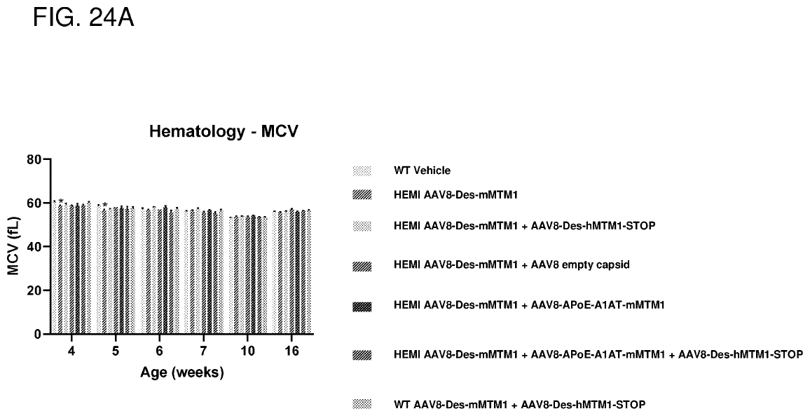

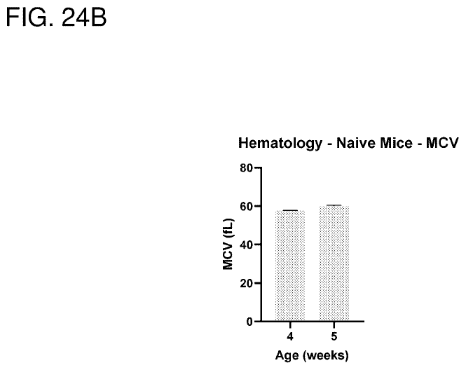

MCV levels were significantly decreased in HEMI AAV8-Des-mMTM1 mice compared to WT Vehicle mice at 4 and 5 weeks of age (t-test, p<0.01, for all) (FIG. 24A).

MCV levels from naïve mice at 4 and 5 weeks of age are shown in FIG. 24B.

### Mean Corpuscular Hemoglobin (MCH)

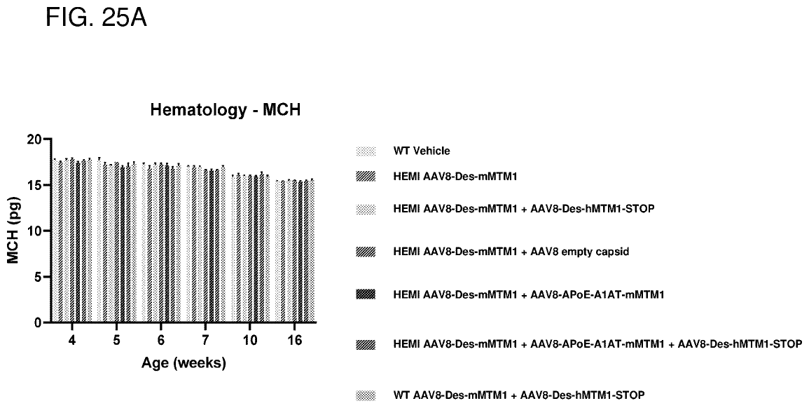

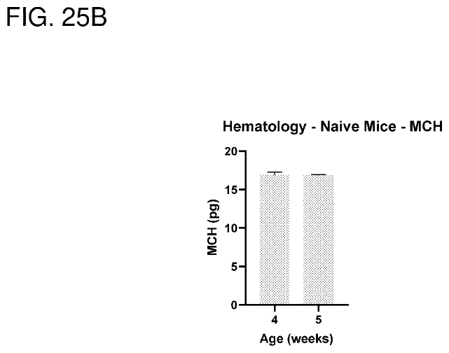

There were no statistically significant differences between the groups in MCH levels (FIG. 25A).

MCH levels from naïve mice at 4 and 5 weeks of age are shown in FIG. 25B.

### Mean Corpuscular Hemoglobin Concentration (MCHC)

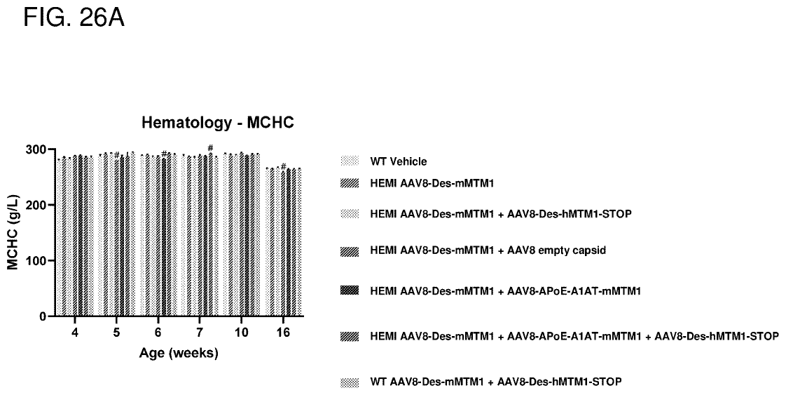

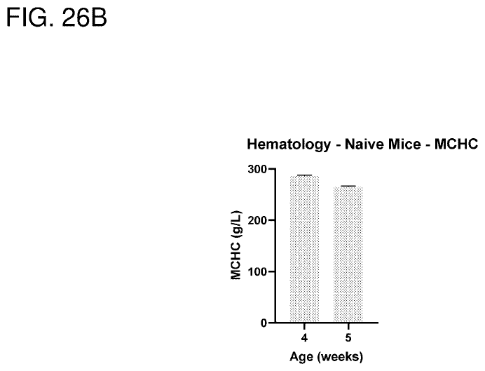

MCHC levels were significantly decreased in HEMI AAV8-Des-mMTM1+AAV8 empty capsid mice at 5 and 16 weeks of age and in HEMI AAV8-Des-mMTM1+AAV8-APoE-A1AT-mMTM1 mice at 6 weeks of age, and increased in HEMI AAV8-Des-mMTM1+AAV8-APoE-A1AT-mMTM1+AAV8-Des-hMTM1-STOP mice at 7 weeks of age compared to HEMI AAV8-Des-mMTM1 mice (Two-Way ANOVA: Group effect: NS, Group×Time interaction effect: p<0.01, Dunnett's post-hoc comparison: p<0.05, for all) (FIG. 26A).

MCHC levels from naïve mice at 4 and 5 weeks of age are shown in FIG. 26B.

### Thrombocyte Count (PLT)

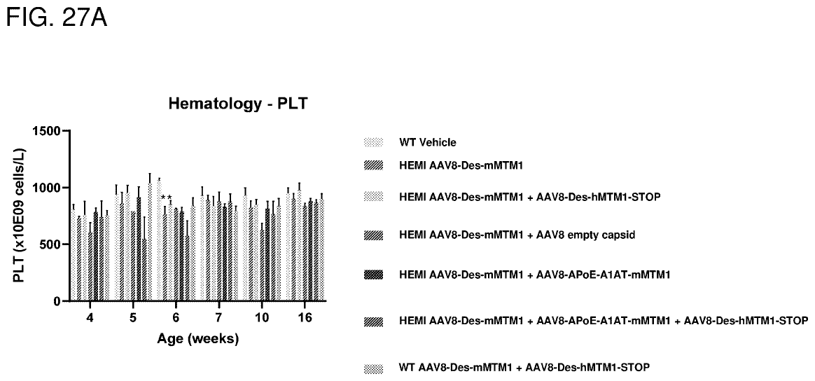

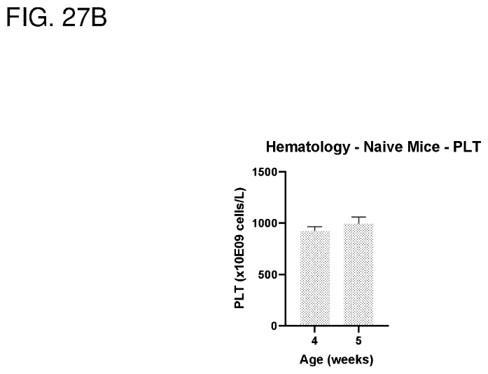

Thrombocyte or platelet counts were significantly decreased in HEMI AAV8-Des-mMTM1 mice compared to WT Vehicle mice at 6 weeks of age (Two-Way ANOVA: Group effect: p<0.05, Dunnett's post-hoc comparison: p<0.01) (FIG. 27A).

Thrombocyte or platelet counts from naïve mice at 4 and 5 weeks of age are shown in FIG. 27B.

### Neutrophil Count (Relative) (Neut(%))

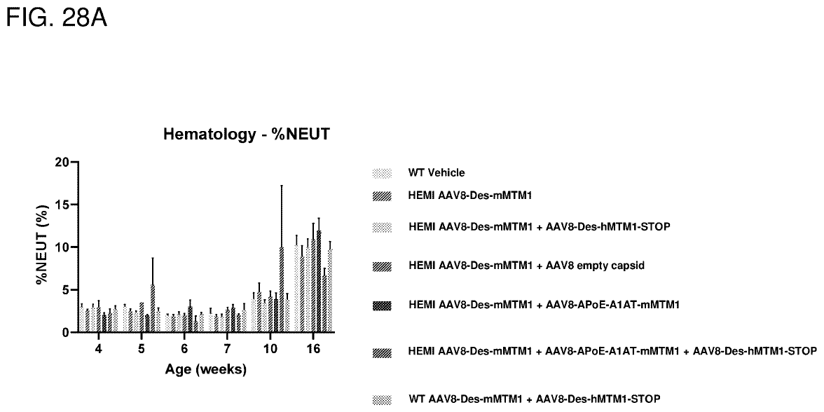

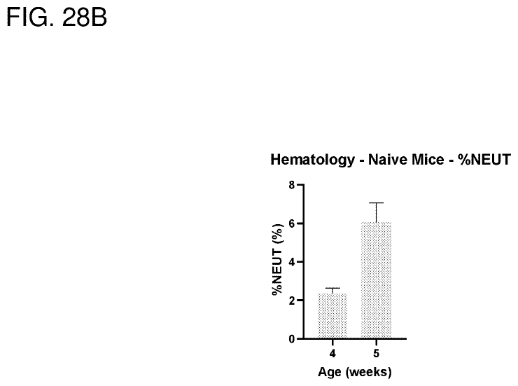

There were no statistically significant differences between the groups in relative neutrophil counts (FIG. 28A).

Relative neutrophil counts from naïve at 4 and 5 weeks of age mice are shown in FIG. 28B.

### Neutrophil Count (Absolute) (Neut)

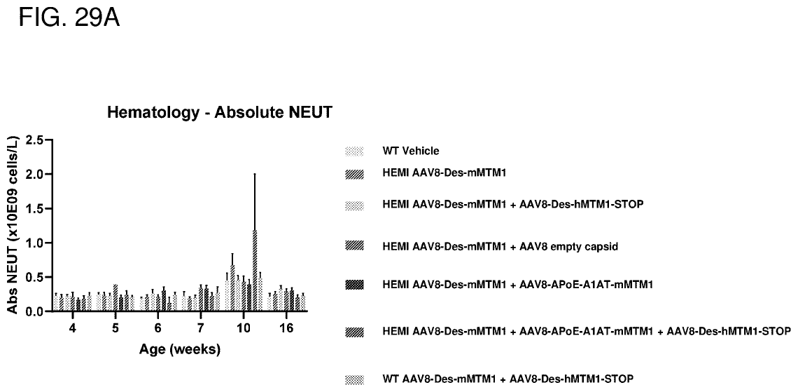

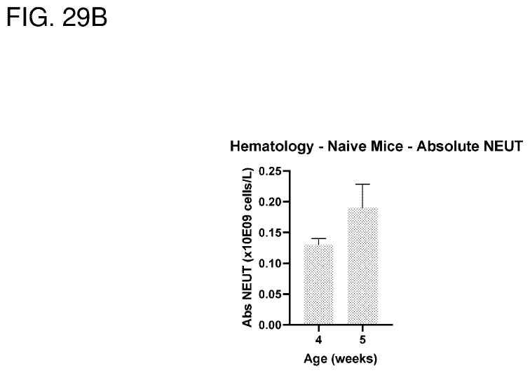

There were no statistically significant differences between the groups in absolute neutrophil counts (FIG. 29A).

Absolute neutrophil counts from naïve mice at 4 and 5 weeks of age are shown in FIG. 29B.

### Lymphocyte Count (Relative) (Lymph(%))

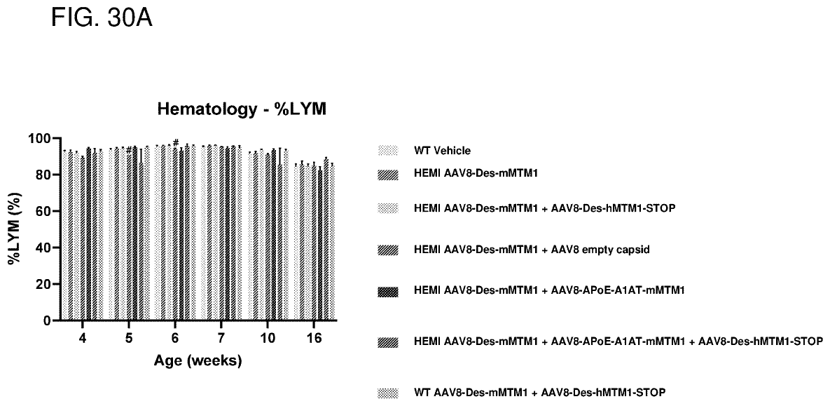

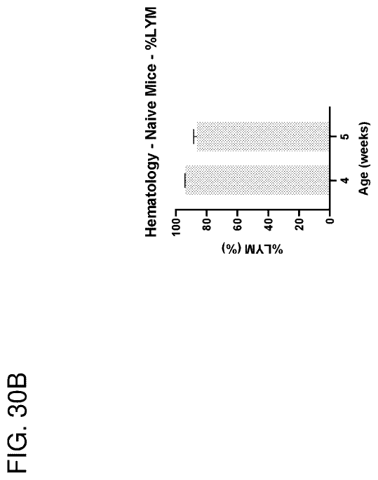

Relative lymphocyte counts were significantly decreased in HEMI AAV8-Des-mMTM1+AAV8 empty capsid mice at 5 and 6 weeks of age compared to HEMI AAV8-Des-mMTM1 mice (Two-Way ANOVA: Group effect: NS, Group×Time interaction effect: p<0.05, Dunnett's post-hoc comparison: p<0.05, for all) (FIG. 30A).

Relative lymphocyte counts from naïve mice are shown in FIG. 30B.

### Lymphocyte Count (Absolute) (Lymph)

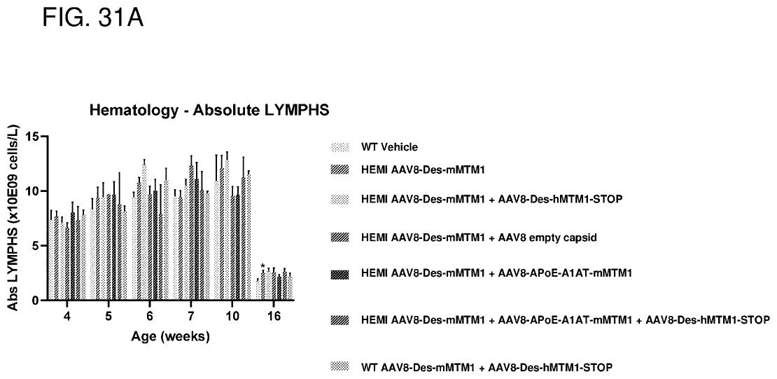

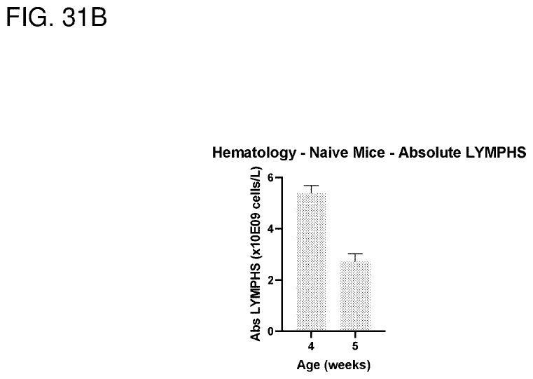

Absolute lymphocyte counts were significantly increased in HEMI AAV8-Des-mMTM1 mice compared to WT Vehicle mice at 16 weeks of age (t-test, p<0.01) (FIG. 31A).

Absolute lymphocyte counts from naïve mice at 4 and 5 weeks of age are shown in FIG. 31B.

### Monocyte Count (Relative) (Mono(%))

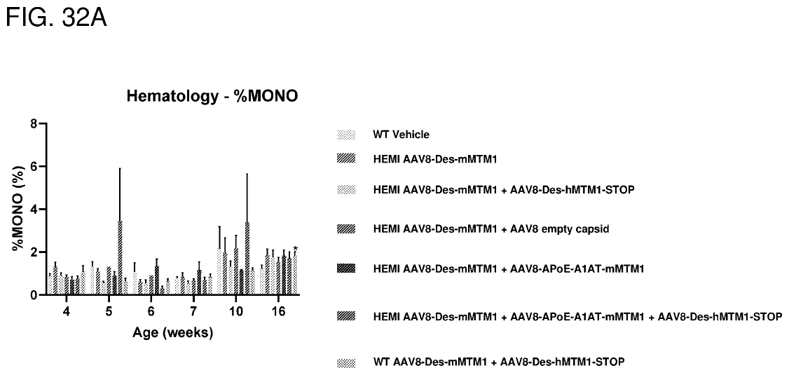

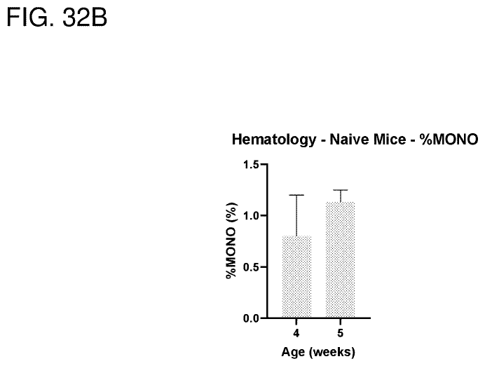

Relative monocyte counts were significantly increased in WT AAV8-Des-mMTM1+AAV8-Des-hMTM1-STOP mice compared to WT Vehicle mice at 16 weeks of age (t-test, p<0.05) (FIG. 32A).

Relative monocyte counts from naïve mice at 4 and 5 weeks of age are shown in FIG. 32B.

### Monocyte Count (Absolute) (Mono)

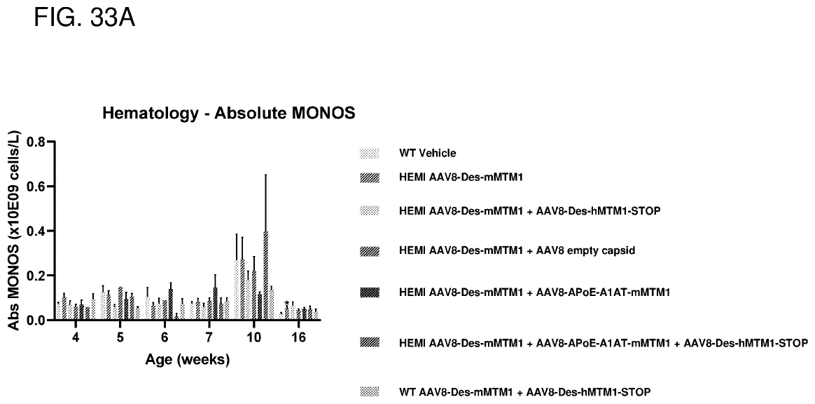

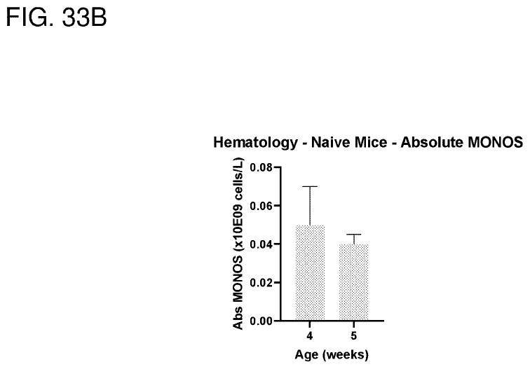

Absolute monocyte counts were significantly increased in HEMI AAV8-Des-mMTM1 mice compared to WT Vehicle mice at 16 weeks of age (t-test, p<0.01) (FIG. 33A).

Absolute monocyte counts from naïve mice at 4 and 5 weeks of age are shown in FIG. 33B.

### Eosinophil Count (Relative) (Eos(%))

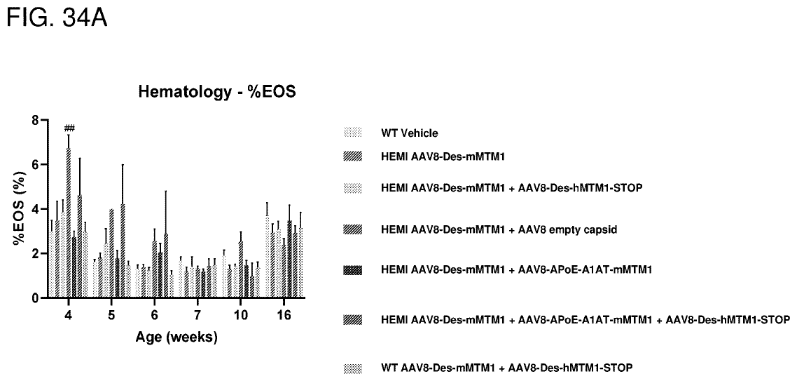

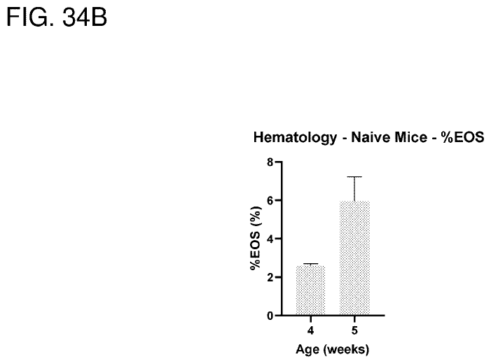

Eosinophil counts were significantly increased in HEMI AAV8-Des-mMTM1+AAV8 empty capsid mice at 4 weeks of age compared to HEMI AAV8-Des-mMTM1 mice (Two-Way ANOVA: Group effect: p<0.05, Dunnett's post-hoc comparison: p<0.01) (FIG. 34A).

Eosinophil counts from naïve mice at 4 and 5 weeks of age are shown in FIG. 34B.

### Eosinophil Count (Absolute) (Eos)

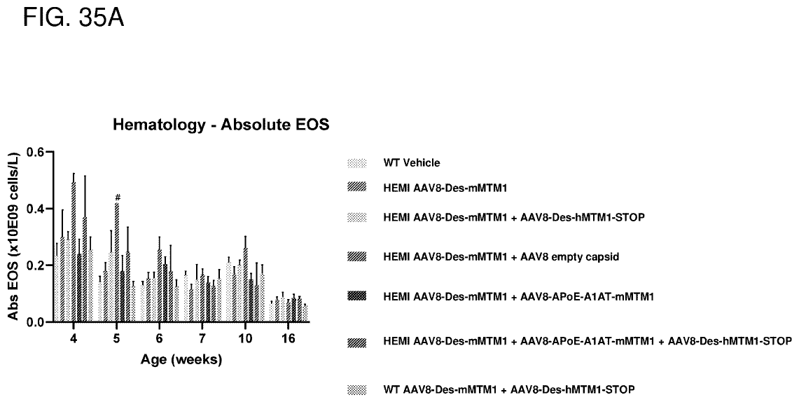

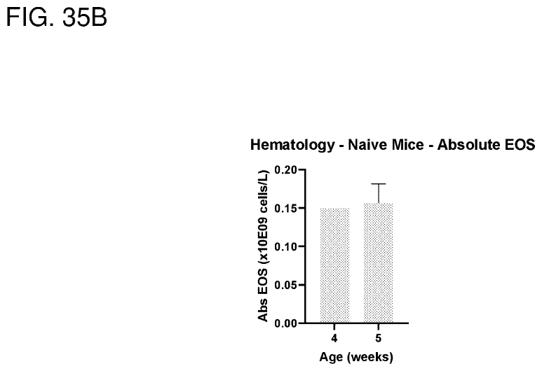

Absolute eosinophil counts were significantly increased in HEMI AAV8-Des-mMTM1+AAV8 empty capsid mice at 5 weeks of age compared to HEMI AAV8-Des-mMTM1 mice (Two-Way ANOVA: Group effect: p=0.0001, Dunnett's post-hoc comparison: p<0.05) (FIG. 35A).

Absolute eosinophil counts from naïve mice at 4 and 5 weeks of age are shown in FIG. 35B.

### Basophil Count (Relative) (Baso(%))

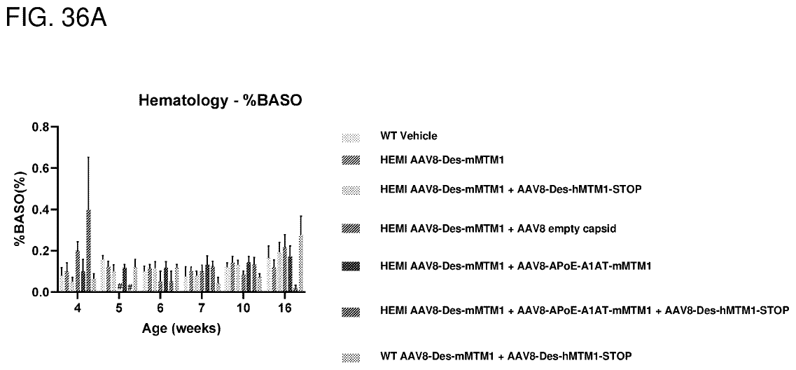

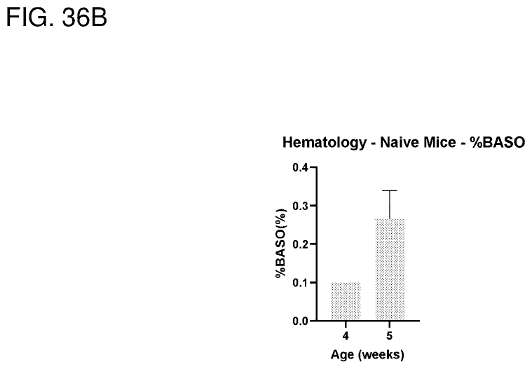

Relative basophil counts significantly decreased in HEMI AAV8-Des-mMTM1+AAV8 empty capsid mice and in HEMI AAV8-Des-mMTM1+AAV8-APoE-A1AT-mMTM1+AAV8-Des-hMTM1-STOP mice at 5 weeks of age compared to HEMI AAV8-Des-mMTM1 mice (Two-Way ANOVA: Group effect: NS, Group×Time interaction effect: p<0.01, Dunnett's post-hoc comparison: p<0.05) (FIG. 36A).

Relative basophil counts from naïve mice at 4 and 5 weeks of age are shown in FIG. 36B.

### Basophil Count (Absolute) (Baso)

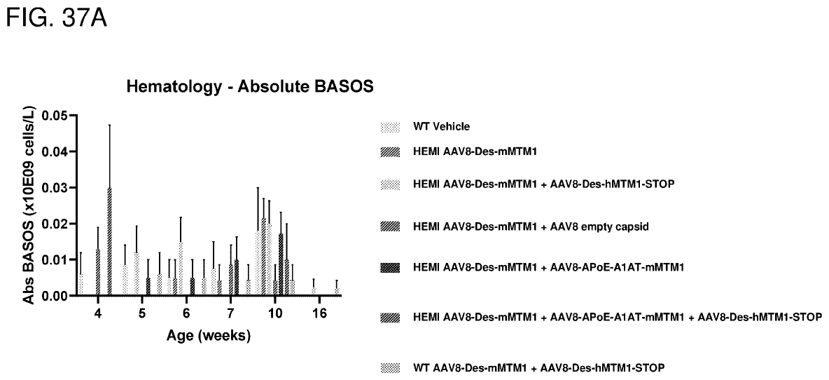

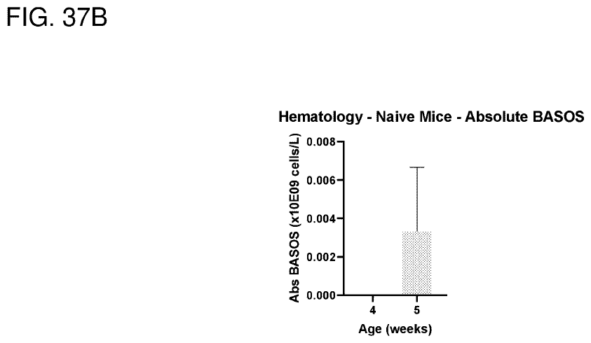

There were no statistically significant differences between the groups in absolute basophil counts (FIG. 37A).

Absolute basophil counts from naïve mice at 4 and 5 weeks of age are shown in FIG. 37B.

### Large Unstained Cell Count (Relative) (LUC(%))

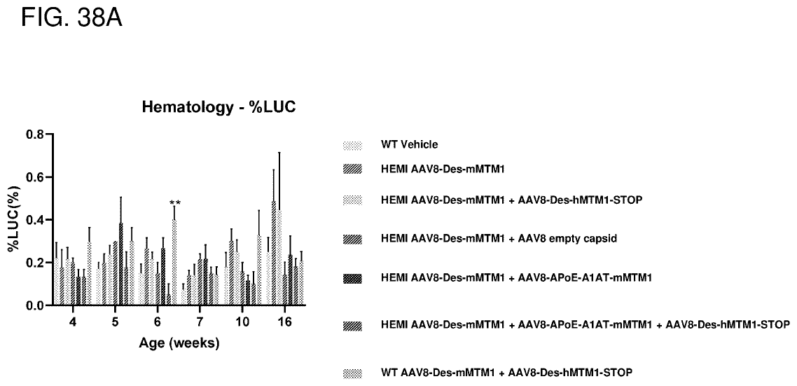

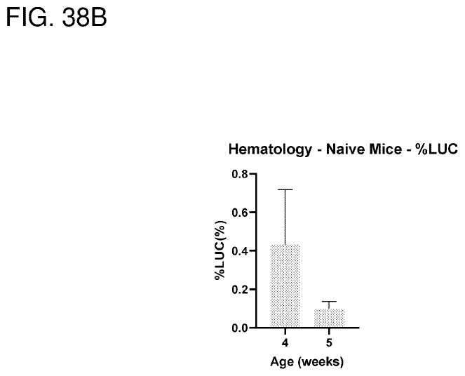

Relative count of large unstained cells was significantly increased in WT AAV8-Des-mMTM1+AAV8-Des-hMTM1-STOP mice at 7 weeks of age compared to WT Vehicle mice (Two-Way ANOVA: Group effect: p<0.05, Dunnett's post-hoc comparison: p<0.01) (FIG. 38A).

Relative count of large unstained cells from naïve mice at 4 and 5 weeks of age are shown in FIG. 38B.

### Large Unstained Cell Count (Absolute) (LUC)

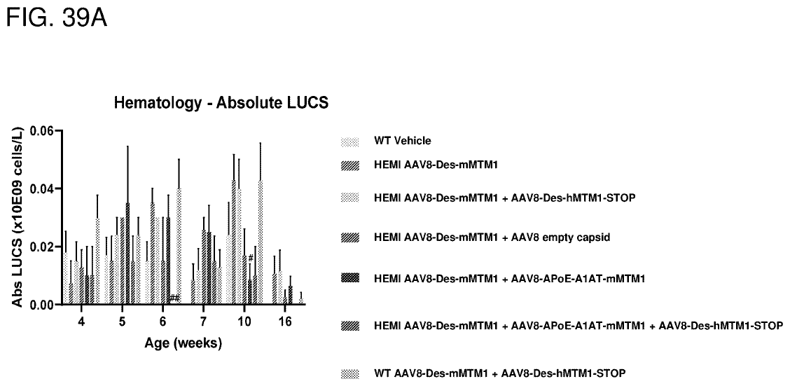

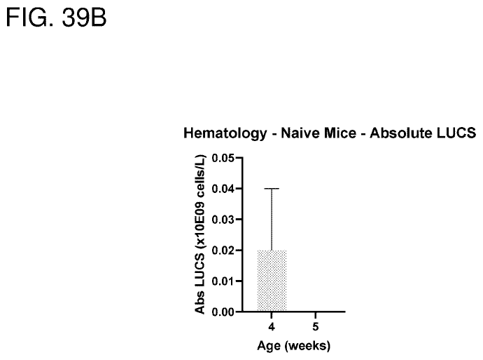

Absolute count of large unstained cells was significantly decreased in HEMI AAV8-Des-mMTM1+AAV8-APoE-A1 AT-mMTM1+AAV8-Des-hMTM1-STOP mice at 6 weeks of age and in HEMI AAV8-Des-mMTM1+AAV8-APoE-A1 AT-mMTM1 mice at 10 weeks of age compared to HEMI AAV8-Des-mMTM1 mice (Two-Way ANOVA: Group effect: NS, Group×Time interaction effect: p<0.05, Dunnett's post-hoc comparison: p<0.05) (FIG. 39A).

Absolute count of large unstained cells from naïve mice at 4 and 5 weeks of age are shown in FIG. 39B.

### Reticulocyte Count (Relative) (Retic(%))

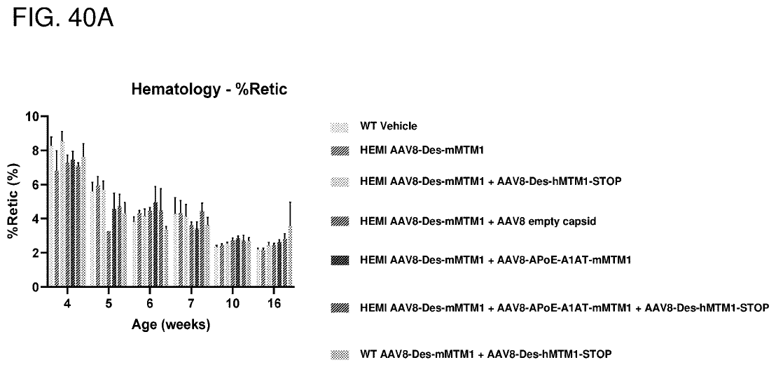

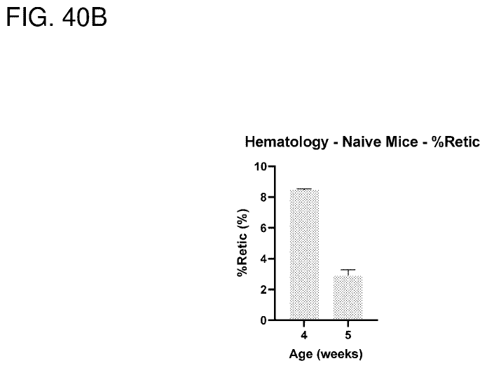

There were no statistically significant differences between the groups in relative reticulocyte counts (FIG. 40A).

Relative reticulocyte counts from naïve mice at 4 and 5 weeks of age are shown in FIG. 40B.

### Reticulocyte Cell Count (Absolute) (Retic)

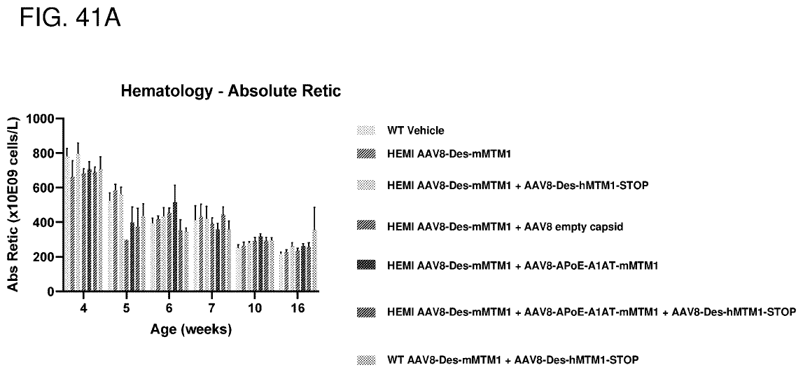

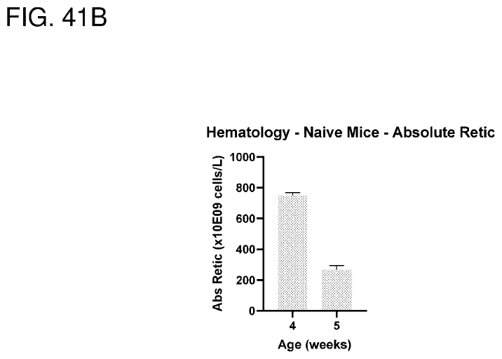

There were no statistically significant differences between the groups in absolute reticulocyte cell counts (FIG. 41A).

Absolute reticulocyte cell counts from naïve mice at 4 and 5 weeks of age are shown in FIG. 41B.

### CONCLUSION

The results of the experiments described above support the safety and efficacy of AAV vectors encoding an MTM1 transgene under the control of a liver-directed regulatory element, optionally in combination with AAV vectors encoding the MTM1 transgene under the control of a muscle-directed regulatory element, for the treatment of XLMTM.

### Example 2. Treatment of X-Linked Myotubular Myopathy in Human Patients by Concurrent Administration of a Pseudotyped AAV2/8 Vector Including a Nucleic Acid Sequence Encoding a Myotubularin 1 Gene Operably Linked to a Liver-Specific Promoter and a Pseudotyped AAV2/8 Vector Including a Nucleic Acid Sequence Encoding a Myotubularin 1 Gene Operably Linked to a Desmin Promoter

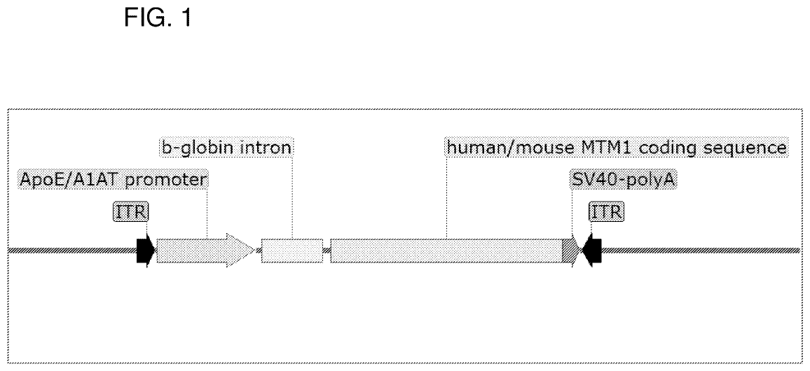

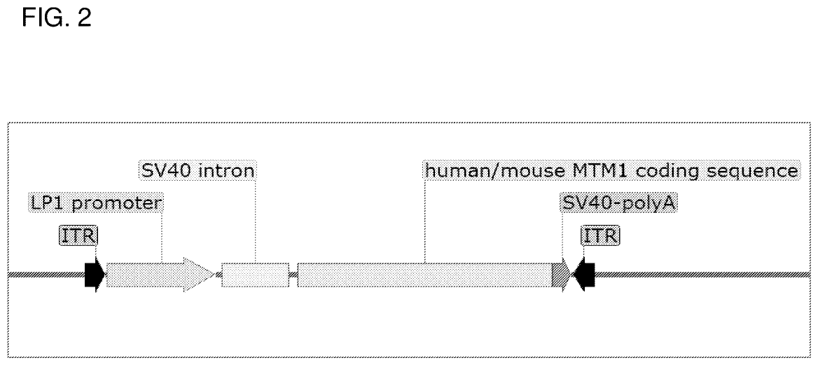

Using the compositions and methods of the disclosure, a patient having XLMTM may be concurrently administered a pseudotyped AAV2/8 vector including a nucleic acid sequence encoding an MTM1 gene operably linked to a liver-specific promoter (FIG. 1 and FIG. 2) and a pseudotyped AAV2/8 vector including a nucleic acid sequence encoding an MTM1 gene operably linked to a desmin promoter (e.g., resamirigene bilparvovec), each in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

Upon concurrently administering the pseudotyped AAV2/8 vector including a nucleic acid sequence encoding an MTM1 gene operably linked to a liver-specific promoter (FIG. 1 and FIG. 2) and the pseudotyped AAV2/8 vector including a nucleic acid sequence encoding an MTM1 gene operably linked to a desmin promoter (e.g., resamirigene bilparvovec) to the patient, the patient displays a change from baseline in maximal inspiratory pressure. For example, the patient displays the change from baseline in maximal inspiratory pressure by about 24 weeks (e.g., by about 20 weeks, 16 weeks, 12 weeks, 8 weeks, or 4 weeks) after concurrent administration of the pseudotyped AAV2/8 vector including a nucleic acid sequence encoding an MTM1 gene operably linked to a liver-specific promoter (FIG. 1 and FIG. 2) and the pseudotyped AAV2/8 vector including a nucleic acid sequence encoding an MTM1 gene operably linked to a desmin promoter (e.g., resamirigene bilparvovec) to the patient.

### Example 3. Treatment of X-Linked Myotubular Myopathy in Human Patients by a Pseudotyped AAV2/8 Vector Including Both a Nucleic Acid Sequence Encoding a Myotubularin 1 Gene Operably Linked to a Liver-Specific Promoter and a Nucleic Acid Sequence Encoding a Myotubularin 1 Gene Operably Linked to a Desmin Promoter

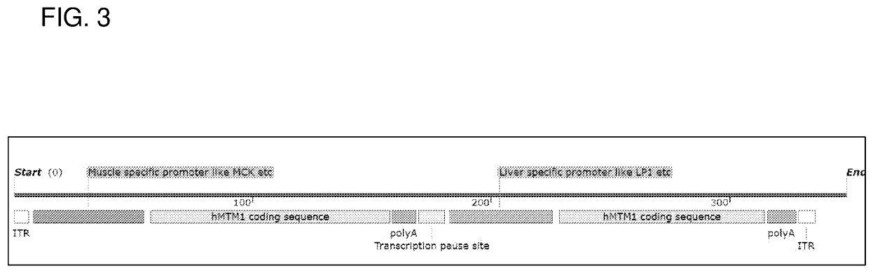

Using the compositions and methods of the disclosure, a patient having XLMTM may be administered a pseudotyped AAV2/8 vector including both a nucleic acid sequence encoding an MTM1 gene operably linked to a liver-specific promoter and a nucleic acid sequence encoding an MTM1 gene operably linked to a muscle-specific promoter (FIG. 3) in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

Upon administering the AAV2/8 vector including both a nucleic acid sequence encoding an MTM1 gene operably linked to a liver-specific promoter and a nucleic acid sequence encoding an MTM1 gene operably linked to a muscle-specific promoter (FIG. 3) to the patient, the patient exhibits a change from baseline in hours of mechanical ventilation support over time. For example, the patient exhibits the change from baseline in hours of mechanical ventilation support over time by about 24 weeks (e.g., by about 20 weeks, 16 weeks, 12 weeks, 8 weeks, or 4 weeks) after administration of the AAV2/8 vector including both a nucleic acid sequence encoding an MTM1 gene operably linked to a liver-specific promoter and a nucleic acid sequence encoding an MTM1 gene operably linked to a muscle-specific promoter (FIG. 3) to the patient.

### Example 4. Treatment of X-Linked Myotubular Myopathy in Human Patients by a Pseudotyped AAV2/8 Vector Including a Nucleic Acid Sequence Encoding a Myotubularin 1 Gene Operably Linked to a Ubiquitous Promoter

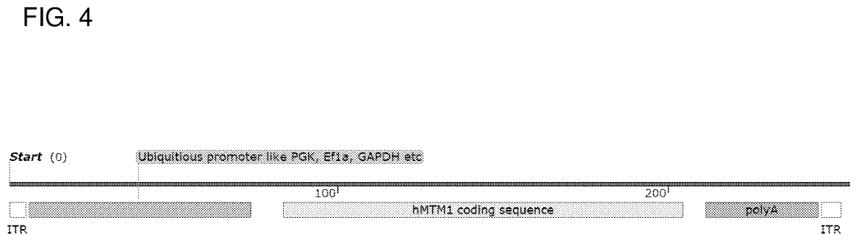

Using the compositions and methods of the disclosure, a patient having XLMTM may be administered a pseudotyped AAV2/8 vector including a nucleic acid sequence encoding an MTM1 gene operably linked to a ubiquitous promoter (FIG. 4) in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

Upon administering the pseudotyped AAV2/8 vector including a nucleic acid sequence encoding an MTM1 gene operably linked to a ubiquitous promoter (FIG. 4) to the patient, the patient achieves functionally independent sitting for at least 30 seconds. For example, the patient achieves the functionally independent sitting by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a ubiquitous promoter (FIG. 4) to the patient.

### Example 5. Treatment of X-Linked Myotubular Myopathy in Human Patients by a Non-Viral Vector Including a Liver-Expressing Construct in Combination with a Pseudotyped AAV2/8 Vector Including a Nucleic Acid Sequence Encoding a Myotubularin 1 Gene Operably Linked to a Desmin Promoter

Using the compositions and methods of the disclosure, a patient having XLMTM may be administered a non-viral vector (e.g. a lipid nanoparticle) including a liver-expressing construct in combination with a pseudotyped AAV2/8 vector including a nucleic acid sequence encoding an MTM1 gene operably linked to a desmin promoter (e.g., resamirigene bilparvovec), with the AAV2/8 vector administered in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

Upon administering the non-viral vector (e.g. a lipid nanoparticle) including a liver-expressing construct in combination with the pseudotyped AAV2/8 vector including a nucleic acid sequence encoding an MTM1 gene operably linked to a desmin promoter (e.g., resamirigene bilparvovec) to the patient, the patient achieves functionally independent sitting for at least 30 seconds. For example, the patient achieves functionally independent sitting for at least 30 seconds by about 24 weeks (e.g., by about 20 weeks, 16 weeks, 12 weeks, 8 weeks, or 4 weeks) after administration of the non-viral vector (e.g., a lipid nanoparticle) including a liver-expressing construct in combination with the pseudotyped AAV2/8 vector including a nucleic acid sequence encoding an MTM1 gene operably linked to a desmin promoter (e.g., resamirigene bilparvovec) to the patient.

1. A recombinant adeno-associated viral (AAV) vector comprising a transgene encoding myotubularin 1 (MTM1), wherein the transgene is operably linked to a promoter that is active in liver tissue.

2. The AAV vector of claim 1, wherein the promoter is selectively active in liver tissue.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The AAV vector of claim 1, wherein the promoter comprises a constitutive promoter.

27. (canceled)

28. The AAV vector of claim 1, wherein the vector further comprises a second transgene encoding MTM1.

29. The AAV vector of claim 28, wherein the second transgene encoding MTM1 is operably linked to a promoter that is active in muscle tissue.

30. (canceled)

31. (canceled)

32. (canceled)

33. (canceled)

34. (canceled)

35. (canceled)

36. (canceled)

37. (canceled)

38. (canceled)

39. (canceled)

40. The AAV vector of claim 1, wherein the AAV is an AAV1, AAV2, AAV3, AAV4, AAV5, AAV6, AAV7, AAV8, AAV9, AAVrh1 0, or AAVrh74 serotype.

41. The AAV vector of claim 1, wherein the AAV vector is a pseudotyped AAV.

42. (canceled)

43. A method of treating X-linked myotubular myopathy (XLMTM) in a human patient in need thereof, the method comprising administering to the patient a therapeutically effective amount of the AAV vector of claim 1.

44. (canceled)

45. (canceled)

46. (canceled)

47. (canceled)

48. (canceled)

49. (canceled)

50. (canceled)

51. The method of claim 43, wherein the patient is further administered an anti-cholestatic agent.

52. (canceled)

53. (canceled)

54. (canceled)

55. (canceled)

56. (canceled)

57. The method of claim 43, wherein the method comprises administering to the patient therapeutically effective amounts of: (i) an AAV vector comprising a transgene encoding MTM1 under the control of a promoter that is active (e.g., selectively active) in liver tissue, and (ii) an AAV vector comprising a transgene encoding MTM1 under the control of a promoter that is active (e.g., selectively active) in muscle tissue.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. A method of treating XLMTM in a human patient in need thereof, the method comprising administering to the patient therapeutically effective amounts of: (i) a non-viral composition comprising a nucleic acid encoding MTM1 operably linked to a promoter that is active (e.g., selectively active) in liver tissue, and (ii) an AAV vector comprising a transgene encoding MTM1 under the control of a promoter that is active (e.g., selectively active) in muscle tissue.

76. The method of claim 75, wherein the non-viral composition is a liposome, vesicle, synthetic vesicle, exosome, synthetic exosome, dendrimer, or nanoparticle.

77. (canceled)

78. (canceled)

79. (canceled)

80. (canceled)

81. (canceled)

82. (canceled)

83. (canceled)

84. A kit comprising: (i) a non-viral composition comprising a nucleic acid encoding MTM1, and (ii) an AAV vector comprising a transgene encoding MTM1 under the control of a promoter that is active (e.g., selectively active) in muscle tissue, wherein the kit further comprises a package insert instructing a user to administer the non-viral composition and the AAV vector to a patient diagnosed as having XLMTM.

85. (canceled)

86. (canceled)

87. (canceled)

88. (canceled)

89. (canceled)

90. (canceled)

91. (canceled)


SEQUENCE LISTING (9 sequences)

SEQ ID NO: 1 - DNA, 12471 bp, synthetic construct
  source 1..12471: mol_type = other DNA
tcgcgcgtttcggtgatgacggtgaaaacctctgacacatgcagctcccggacgtcattgtcgatcctgcaggcgtacggtaaaaaaaggcatagctaacaaggtgtggaaaaagaattagtggttagagagtgagctattcgttgaaacaattgcgttcttgaaacaattcttgctggtaaaatgtcacattttatgtgactacaggtggaggattggcacataacctaaccagtgggggaaacaattgacctctggatttgtccaagtgtatagtagcatttgcccaatcgaatggtcctggtaaggtgttaatgttgactagaaccaaaggtggaagttgcagggaaactggtttagtacaagggtggacaccaggcagtcatccagaggcccattaaaggccttggaatgtttttccgaaggagaatcactccctcttctctcgcttaaagttttaggggattcatgaacagctgctgtgggatagtttcatgtccctagcaattgtaaagcaactgagggtggcttaaaccagttttagctttagggttagggttactggactaaaatttgagaaattcataaatcttaaggaaatccattgtgagttttcattatgagtgcatccaatgtataatttccatgaccctcccatgcaagtgagcatgtgaatcaggaaacgttacaagaacccaacaaactcaaccactactagacaggcgatcacttccagttagtatgcaactttctgtgtaattttagttaccattaaaatctggatgaccttagtgtaaggaaaaaataccttgaatagtgttaaagatgtacacttggtgtcaggcattgtaacattgataaatctgtgtaaggtgctttttgaaaacttcaaagctgcatcaagtcaagtacaagaaaggccatggctgctaaagctgttgaagatgtgggatggaactgggtcacattggtgttaacagcgttgtgcagagccggcaggatcttggtgtgagcgaacattagtctatttaataaagctgtgtgaatgttgtagaggtgaggatgctcacttgaaaactcactgaagaacacttggccccttgaactaaagtgcttctatcaagttcagtgagaaattccgaattacaagcataggtactagaaaagttttgaaaagcagtatagagcaacataagcacattcataaaattagtgatgtagaaagtgaaatttccacgtatggtcactcccagagaaaaaaaatacgtttatttaccttttttaaaaataggggatttcaggccgggtgaggtggctcacgcctgtaatcccagcactttgggaggcccaggtgggcggatcacctgaggtcaggagttggagggatggcaaatcccatctctacaaaatatacaaaaaaatagctgggtgtgttggcaggcgcctgtaatcccagctactcggaaggctgaggcaggagaatccctggaaccagggatgtggaggttgcagtgagccgagattgtgtaactgcattccagcctgggcaacaagagcaagactccgtatcaggaaaaaaaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacaagatttctattttaattggattcttagggctaataaaataatcagccttagcacttatttatttattttttttgagagggagtctcgctctgttgtccatgctggagtgcagtggcgtgatctcggctcactgcaagctccacctcatgagttcacaccattctcctgcctcagtctcccgagtagctgggactccaggcgccctctacaaagcccgtctaattttttttgtatttttagtagagacagggtttcactgtgttagccaggatggtcttgatctcctgaccttgtgatctgcccgcctcggcctcccaaagtgctgggattataggcttgagccactgctcccggccagcacttatttttataattcttcatgattactgtgttactgtcccatgggccgccagggccagctaggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcctagcacgcgctaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccggactctagaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgcaagtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgaccggtccgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgaccggccgctctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctagaggccgccagggccatatttctcaatttttaaatttttcaaaaaaattaatccttaatgtgcatatttttgaattgttaatataactttttgaggtgatgtcttcatgtgtttcaactacttaaaaacttttaaacagtatataataaaaaatcttccaggccactcacacctgtaatcccagcactttgggaggctgaggtgggcagatcacctgagggcaggagttcgagaccagcctggccaatatatatatattcatatattcatatatatatatatattcatatattcatatatatatattcatatattcatatatatatatatatatatatatagcaaaacctcatctctaataaaatacaaaaattagctgagcgtggtgatggatgcctgtagtcccagctactcgggaggctgaggcaggagaatctcttgaacctgggaggtggaggttgcagtgagctgagatggtgccactgccctccagcctgagtgacagagcgagactcggtctccaaaaaaaaacaacaaaaaaatcttccatccttgtctcccatccaccccttccccccagcatgtacttgcagactttatgcatatacagtgagtactgtatatacacaaataataaaaaaatcatatatataatatatgtaattcccctttacatgaaaggtagcacactggtctgtacagtctgtctgcactgtgctatttcactttatatttttatagtttgacagagttctaacatttctttttttttttttttaacagagtcttgttcctgattgttaaattttaaagcatcctaaagtttggtttcacacttgaatgaataccatgtaaggattcacttacatagatgtggttgcctgaatcttaagaataaaataacattgtttgtatttatttaaattagtgttccttttatggtttgcctgaaagcacaacaaaatcctcaccaagatattacaattatgactcccatacaggtaaactgtttagagattggcaagcaccttttaatgaaaggagtcagccagcttagtgtgcagtatttatttctgccggaagagggagcttcagggacagactttggtttagtcatgaagcctccagcactcccaagcggttgtggttgaccaagcaatttatgcttttacctttctacttccagaggcttgtttacttatcagtaagcattaatttagtgtcccctcagatgccttttactttcttcttttctgcctagaataagctgctcttccaattttgcagctacatgtttccaccccagttggaatttctccataacatccattgtagctatccttcaatctacagcctctatttcctgttatagctggtcaggtctaatccctcaaaatactctgtcccctgcttcccttatctgctggccacctttttcccccacatacacactgccatgtcccacccttcactcaagttgttccctgccacctcaacaaatttaagtccataaaatagagtaagtgttcctgactgttaaattttaaagcatcccaaagtctgatttcacactcgaatgaatactatgtacggattcatttacatagatgcggttgcatgagtcttaacaaaaaaataacattatttgtatttattcaaagtactgtcaagatataatgtcaagacctaattcaaaggttccacaaagccttccttgactgcccccaacgaagattatccattttccctgaaatcccattgacttttctattttgtaaggaggctcgtgagactctgtctaaaaacaaaacaaaacaaaaagaaacaatcaaacggcttgcttctgttctttgatctgctagtaagcaaaaattacacatggtgacaggagctatgtgaggctgtcaggttgaatgggaggagtttgggatcctgcttgtggatggttggaagaggctttcgggaaagacagtatttatgtgagacctggaagatgggccttagctttgcagaaggtggagaggcaggaaatagcacgggggccctggggctggaagacttgggcatatttgaggaacagaaaggagaccagcataactgaggtgggaaaagcatgtgaagagatggggctggaggaggccgggagtggtggctcacgcctgtaatcccagcactttgggaggccaaggcaggcggatcatgagctcaggagattgagaccatcctggctaacacggtgaaaccccctctctactaaaaatacaaaaaaaaaaaaaaaaaaaattagctgggcgtggtggcaggagcctgtagtcccagctacctgggaggctgaggcaggagaatggcgtgaacctggaaggctgagcttgcagtgagccgagattgcaccactgcactccagcctgggagacagagagagactccctctcaaaaaaacaaacaaacgaaacaaaacaaaacaaaaattagccaggcgtggtggtatgcacctgtaatcccagctactcgggaggttgaggcaggagaaacgcttgaactcaggaggcggaggttgcagtgagccgagactgcgccactgcactccagcctgggtgacagagggagactccatctcaaaaaaaaaaatttttttttttttacaaacggtgtctccctctgtcgcccaggctggagtgcagtggtgtgatcacagctcactccagcctcaacctccccagctgaagccatcctcttgcctcagcctcctaagtagctgggactacaggcgcgcacctccaggcttggctcttattctttttattgtttttgaaactatagaacctatttttaaaaaatgttttggttgtttttattgctgcttttccttttggggttagaacacaagttttgatgggaaacaggttagaacacattcatctcttcccatagcgatggtcatagaaaaacggggcatatttataaactctcagttgatcttaaaatgtgcaaaagctgccgaactcctgggagtgagctcgagccctgcaggatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 2 - DNA, 775 bp, synthetic construct
  source 1..775: mol_type = other DNA
cgtgatctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcctcgacggtatcgataag

SEQ ID NO: 3 - DNA, 449 bp, synthetic construct
  source 1..449: mol_type = other DNA
gtcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtgcacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 4 - DNA, 66 bp, Simian virus 40
  source 1..66: mol_type = genomic DNA; note = simian DNA tumor virus (SV40)
aggtaaatataaaatttttaagtgtataatgtgttaaactactgattctaattgtttctctctttt

SEQ ID NO: 5 - DNA, 476 bp, Homo sapiens
  source 1..476: mol_type = genomic DNA
agggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctccca

SEQ ID NO: 6 - DNA, 1812 bp, Homo sapiens
  source 1..1812: mol_type = genomic DNA
atggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctga

SEQ ID NO: 7 - DNA, 1812 bp, Mus musculus
  source 1..1812: mol_type = genomic DNA
atggcttctgcatcagcatctaagtataattcacactccttggagaatgaatccattaagaaagtgtctcaagatggagtcagtcaggatgtgagtgagactgtccctcggctcccaggggagttactaattactgaaaaagaagttatttacatatgtcctttcaatggccccattaagggaagagtttacatcacaaattatcgtctttatttaagaagtttggaaacggattctgctctaatacttgatgttcctctgggtgtgatatcaagaattgaaaaaatgggaggcgcgacaagtagaggagaaaattcctatggtctagatattacttgtaaagatttgagaaacctgaggtttgcattgaagcaagaaggccacagcagaagagatatgtttgagatccttgtaaaacatgcctttcctctggcacacaatctgccattatttgcatttgtaaatgaagagaagtttaacgtggatgggtggactgtttataatccagttgaagaatatagaaggcagggcctgcccaatcaccattggaggataagttttattaacaagtgctatgagctctgtgagacataccctgctcttttggtggttccctatcggacctcagatgatgatcttaggaggatcgcaacgtttagatcccgaaatcggcttcctgtactgtcgtggattcacccagaaaacaaaatggtcattatgcgctgcagtcagcctcttgtcggtatgagtggtaaaagaaataaagatgacgagaaatacctggatgtgatcagggaaactaacaaacaaacttctaagctcatgatttatgatgcacgacccagtgtaaatgcagtcgccaacaaggcaacaggaggaggatatgaaagtgatgacgcatatcaaaactcagaactttccttcttagacattcataatattcatgttatgcgagaatctttaaaaaaagtgaaagatattgtttatcccaacatagaagaatctcattggttgtccagtttggagtctactcattggttagaacatatcaagcttgttctgaccggtgccattcaagtggcagaccaagtgtcttcaggaaagagctcggtacttgtgcactgcagtgacggatgggacaggaccgctcagctgacatccttggccatgctgatgttggacagcttctacagaactattgaaggctttgagatattggtacagaaagagtggataagttttggccataaatttgcatctagaataggtcatggtgataaaaaccatgctgatgctgatcgatctcctatttttcttcagtttattgactgtgtgtggcagatgtcgaaacagttccccacagcttttgagttcaatgaaggctttttgattaccgttttggatcatctgtatagctgtcgatttggtactttcttattcaactgtgactcggctcgagaaagacagaaacttacagaaagaacagtttctctatggtcgctaattaacagcaataaagacaaattcaaaaaccccttctatacaaaagaaatcaatcgggttttgtatccagttgccagcatgcgtcacttggaactgtgggtgaattattacatccgatggaatcccagggtcaagcagcaacagcccaacccagtggagcagcgttacatggagcttttggccttgcgtgacgattatataaagaggctcgaggaattgcagctggccaactccgccaagcttgctgatgcccccgcttcgacttccagttcgtcacagatggtgccccatgtgcagacgcacttctga

SEQ ID NO: 8 - DNA, 193 bp, Homo sapiens
  source 1..193: mol_type = genomic DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggg

SEQ ID NO: 9 - DNA, 254 bp, Homo sapiens
  source 1..254: mol_type = genomic DNA
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat